(19) United States
(12) Patent Application Publication
(10) Pub. No.: US 2020/0405883 A1
(43) Pub. Date: Dec. 31, 2020

(54) METHOD OF TREATMENT WITH VIRAL-BASED GENE THERAPY

(71) Applicants: Baxalta Incorporated, Bannockburn, IL (US); Baxalta GmbH, Glattpark (Opfikon) (CH)

(72) Inventors: Ivan BILIC, Vienna (AT); Birgit Reipert, Deutsch Wagram (AT)

(21) Appl. No.: 16/906,349

(22) Filed: Jun. 19, 2020

Related U.S. Application Data
(60) Provisional application No. 62/867,172, filed on Jun. 26, 2019.
(60) Provisional application No. 62/864,404, filed on Jun. 20, 2019.

(51) Int. Cl. A61K 48/00; A61K 39/395; C07K 16/24; C07K 16/28

(52) U.S. Cl. CPC A61K 48/005; C07K 16/2866; C07K 16/248; A61K 39/3955

(57) ABSTRACT

Methods are provided for treating a patient with viral-based gene therapy that promote persistent transgene expression. The methods include administering to the patient an inhibitor of the interleukin-6 (IL6) signaling pathway or the NCOR2/SMRT histone deacetylation pathway, and a viral-based gene therapy vector. Methods are also provided for assigning viral-based gene therapy to a patient that include determining whether the patient has a genotype sensitizing the patient to persistent infection by a viral-based gene therapy vector by evaluating whether the patient has a mutation in the SMRT/NCOR2 gene associated with reduced SMRT/NCOR2 protein function or in the interleukin-6 receptor (IL-6R) gene associated with reduced IL-6R function. If the patient has either a mutation in the SMRT/NCOR2 gene associated with reduced SMRT/NCOR2 protein function or a mutation in the IL-6R gene associated with reduced IL-6R function, a viral-based gene therapy vector is assigned to the patient.

### CROSS REFERENCE TO RELATED APPLICATIONS

This application claims priority to U.S. Provisional Patent Application No. 62/864,404, filed Jun. 20, 2019, and U.S. Provisional Patent Application No. 62/867,172, filed Jun. 26, 2019, the contents of which are hereby incorporated by reference, in their entireties, for all purposes.

### STATEMENT OF FUNDING

This invention was made in part with Government support under Grant Number NIH NHLBI RC3 HL103396-01 awarded by the National Institutes of Health. The Government may have certain rights in the invention.

### BACKGROUND

Several clinical studies have demonstrated the use of DNA virus vectors, derived from the nonpathogenic adeno-associated virus (AAV), for successful delivery of transgenes into patients in need of gene therapy, such as those suffering from with hemophilia B. An AAV vector construct is produced by replacing viral genes with a therapeutic cassette consisting of a promoter, the transgene of interest and poly A tail. Although functional transgene expression has been achieved in patients, maintenance of effective plasma levels of transgene-derived protein or enzyme has been hypothesized to be limited by immune responses (IR) directed at the AAV vector, leading to elimination of successfully transduced hepatocytes. See. Nathwani A C et al., The New England Journal of Medicine, 371:1994-2004 (2014) and Nathwani A C et al., The New England Journal of Medicine, 365:2357-65 (2011).

In some cases, asymptomatic increases in alanine aminotransferase (ALT) levels observed in some patients at 6-10 weeks post AAV vector infusion were successfully treated with corticosteroids, which enabled maintenance of activity levels in blood of the transgene-derived protein or enzyme. See, Nathwani A C et al., Hum. Gene Ther., 28:1004-12 (2017). Nathwani A C et al., (2014) Supra, and Nathwani A C et al., (2011) Supra. Transaminitis and the instability of transgene expression were found to be associated with vector dose-dependent production of circulating cytotoxic T cells (CTLs) recognizing the AAV capsid, leading to the conclusion that adaptive CTL IRs might be responsible for the clearance of transduced hepatocytes and the loss of transgene expression. Nathwani A C et al., (2017) Supra, Nathwani A C et al., (2014) Supra, and Nathwani A C et al., (2011) Supra.

One of the conditions that are amenable to be treated by gene therapy is hemophilia B. Hemophilia B is a bleeding disorder caused by deficient activity of blood coagulation factor IX (FIX), resulting from mutations in the F9 gene on the X chromosome. The bleeding tendency seen in patients has a variable severity related to circulating FIX levels: <1% of normal activity (severe disease) is associated with spontaneous bleeding, commonly into joints and muscles, whereas >5% to 40% activity (mild) is associated with rare spontaneous bleeding and better preservation of joint function. Relatively modest increases in clotting factor levels, to between 15-20%, are thought to be sufficient to protect against joint bleeding and its associated debilitating effects in these patients.

Current standard therapy for hemophilia B involves intravenous infusions of replacement FIX clotting factor concentrates as prophylaxis or to treat bleeding episodes. This has several disadvantages, including the cost and inconvenience of frequent prophylactic infusions (up to 3 times per week), the need to plan physical activity around peaks and troughs in clotting factor levels, and the possibility of break-through bleeds. In spite of management with replacement therapy, hemophilia B continues to have a considerable negative impact on the daily lives of sufferers.

Gene therapy is being studied as a potentially curative approach to maintaining effective circulating FIX levels in these patients, by delivering functioning human FIX genes into liver hepatocytes. Likewise, methods of treating patients with a viral-based gene therapy that result in persistent gene expression are being studied.

### SUMMARY

Accordingly, there is a need in the art for methods that improve conventional gene therapy. Specifically, there is a need for methods that improve the length of transient transgene expression in non-integrative viral vectors, such as adenoviruses. The present disclosure solves these and other problems in the art, for instance, by providing methods of facilitating sustained transgene expression upon AAV viral-based gene therapy, e.g., through concomitant suppression of the IL6 signaling pathway and/or the NCoR2/SMRT deacetylation pathway.

In some embodiments, the disclosure provides a method for treating a patient with viral-based gene therapy, which includes administering to the patient an interleukin-6 (IL6) pathway inhibitor and a viral-based gene therapy vector. In some embodiments, the IL6 pathway inhibitor is an inhibitor of IL6 or an inhibitor of the interleukin-6 receptor (IL6R). In some embodiments, the IL6 pathway inhibitor is an anti-IL6 or an anti-IL6R monoclonal antibody.

The present disclosure also provides methods for identifying patients who are likely to respond more favorably to gene therapy, e.g., adenoviral-based gene therapy. In some embodiments, these methods include determining whether a patient has a genotype sensitizing the patient for persistent viral-based gene therapy. In some embodiments, the method evaluates whether the subject has a mutation that suppresses the IL6 signaling pathway and/or NCoR2/SMRT deacetylation pathway.

In some embodiments, such a method includes determining whether a patient has a genotype sensitizing the patient to persistent infection by a viral-based gene therapy vector, and in response to a determination that the patient has the said genotype, administering a viral-based gene therapy vector to the patient.

In some embodiments, determining whether the patient has the said genotype includes evaluating one or both of whether the patient has a mutation in the SMRT/NCOR2 gene associated with reduced SMRT/NCOR2 protein function, and whether the patient has a mutation in the interleukin-6 receptor (IL-6R) gene associated with reduced IL-6R function.

In some embodiments, the genotype sensitizing the patient to persistent infection by a viral-based gene therapy vector comprises a mutation in at least one copy of the patient's IL-6R gene that causes IL-6R haplodeficiency. In some embodiments, the mutation in the at least one copy of the patient's IL-6R gene is a missense mutation in the IL-6R gene.

In some embodiments, the genotype sensitizing the patient to persistent infection by a viral-based gene therapy vector comprises mutations in both copies of the patient's SMRT/NCOR2 gene that reduce the protein function of the encoded SMRT/NCOR2 proteins by at least 75%, relative to wild type SMRT/NCOR2 protein function.

In some embodiments, the patient is a subject in need of treatment for a disease associated with insufficient level of an enzymatic activity.

In another aspect, the present disclosure provides a method for treating disease associated with insufficient level of an enzymatic activity. The method includes determining whether a patient has a genotype sensitizing the patient to persistent infection by a viral-based gene therapy vector, and in response to a determination that the patient has the said genotype, administering a viral-based gene therapy vector to the patient.

In some embodiments, upon determination that the patient does not have the said genotype, the method includes administering a protein therapeutic having the enzymatic activity to the patient.

In some embodiments, determining whether the patient has the said genotype includes evaluating one or both of whether the patient has a mutation in the SMRT/NCOR2 gene associated with reduced SMRT/NCOR2 protein function, and whether the patient has a mutation in the interleukin-6 receptor (IL-6R) gene associated with reduced IL-6R function.

In some embodiments, the genotype sensitizing the patient to persistent infection by a viral-based gene therapy vector comprises a mutation in at least one copy of the patient's IL-6R gene that causes IL-6R haplodeficiency. In some embodiments, the mutation in the at least one copy of the patient's IL-6R gene is a missense mutation in the IL-6R gene.

In some embodiments, the genotype sensitizing the patient to persistent infection by a viral-based gene therapy vector comprises mutations in both copies of the patient's SMRT/NCOR2 gene that reduce the protein function of the encoded SMRT/NCOR2 proteins by at least 75%, relative to wild type SMRT/NCOR2 protein function.

In some embodiments, the viral-based gene therapy vector is an adeno-associated virus (AAV) vector.

In some embodiments, the viral-based gene therapy vector comprises a polynucleotide having a nucleic acid sequence encoding a therapeutic protein, wherein the nucleic acid sequence encoding the therapeutic protein comprises at least 10 CG dinucleotides.

In some embodiments, wherein the patient has hemophilia A and the viral-based gene therapy vector encodes a Factor VIII polypeptide. In some embodiments, the encoded Factor VIII polypeptide is a B-domain deleted Factor VIII polypeptide. In some embodiments, the encoded Factor IX polypeptide has an R338L amino acid change relative to the wild type Factor IX sequence.

In some embodiments, the viral-based gene therapy vector comprises a Factor VIII polynucleotide encoding the Factor VI polypeptide, the Factor VIII polynucleotide comprising the nucleic acid sequence of SEQ ID NO: 1 [CS04].

In some embodiments, the viral-based gene therapy vector comprises a Factor VIII polynucleotide encoding the Factor VIII polypeptide, the Factor VIII polynucleotide comprising the nucleic acid sequence of [CS04+NG5+X5].

In some embodiments, the viral-based gene therapy vector comprises a Factor IX polynucleotide encoding the Factor IX polypeptide, the Factor IX polynucleotide comprising the nucleic acid sequence of SEQ ID NO: 7 [CS06].

Other objects, advantages and embodiments of the invention will be apparent from the detailed description following.

### BRIEF DESCRIPTION OF THE DRAWINGS

FIG. 1 shows baseline patient characteristics for the study described in Example 1.

FIG. 2 shows Identification of variants potentially impacting FIX transgene expression in patient 5 by whole exome sequencing, as described in Example 1.

FIG. 3 illustrates an example Factor IX gene therapy construct. FIX gene therapy construct contains a self-complementary adeno-associated virus (scAAV) genome consisting of a truncated 320 base pair (bp) murine transthyretin (TTR) promoter/enhancer, followed by a 77 bp intron fragment from minute virus of mice (MVM), the codon optimized FIX Padua (R338L) cDNA transgene, and a bovine growth hormone (BGH) polyadenylation sequence. This expression cassette is flanked by one wild type 145 nt AAV2 inverted terminal repeat (ITR) sequence and another ITR (modified inverted terminal repeat [ΔITR]) with an engineered deletion in the AAV DNA resolution site and D sequence, so as to direct preferential replication and packaging of self-complementing rather than conventional single-stranded AAV DNA sequences.

FIGS. 4A, 4B, 4C, 4D, 4E, 4F, 4G, 4H, 4I, 4J, 4K, 4L, 4M, and 4N collectively illustrate FIX activity, liver enzyme and AAV8 IFN-γ ELISPOT data for patients in each dose cohort, as described in Example 1. Factor IX activity post FIX gene therapy construct infusion by patient and dose cohort plotted in relation to bleeding episodes, administration of FIX replacement therapy and prednisolone dosing (patients 6, 7 and 8) as well as liver enzyme ALT and AST markers of hepatotoxicity. Patients 6 and 7 received prednisolone as a rescue treatment in response to a sudden loss of FIX expression. Patient 8 received prophylactic treatment with prednisolone. The duration of prednisone treatment is shown in the blue bar chart, with the slope indicating dose tapering. The results from IFN-γ ELISPOT assays are shown in the lower graph panel for each patient; the reaction of the patient's PBMCs to AAV8 capsid peptides are plotted as the number of spot-forming units per 1 million PBMCs in relation to the control (red line). Patient 8 was treated with the intermediate dose of FIX gene therapy construct and an accompanying regimen of prophylactic corticosteroids. FIX activity levels of 2-3% were attained for the first 6 months of the study in the absence of elevated liver transaminases or T cell activation, consistent with the other patients in cohort 2. The patient developed squamous cell carcinoma of the tonsil during month 7 requiring treatment, and returned to regular prophylactic FIX infusions. AAV8, adeno-associated virus serotype 8; ALT, alanine aminotransferase; AST, aspartate aminotransferase; FIX, factor IX; IFNγ ELISPOT, interferon-γ enzyme-linked immunosorbent spot; PBMC, peripheral blood mononuclear cells.

FIG. 5 illustrates peak FIX:C by patient and dose cohort, in the study described in Example 1. Peak FIX:C activity measured in each patient after receiving a single infusion of FIX gene therapy construct in 1 of 3 ascending dose cohorts.

FIG. 6 illustrates Anti-AAV8 NAb responses in mice treated with FIX gene therapy construct versus CpG-depleted vector constructs, as described in Example 1. Anti-AAV8 NAb responses, as a surrogate marker for immune activation via TLR9, indicate a lower immunogenicity by CpG depleted vectors. C56Bl/6 mice (8-10 weeks old, n=6-8 per group) were treated intravenously with identical doses (4×10¹² vg/kg) of the scAAV8 vector each carrying a FIX Padua coding sequence containing different numbers of CpG oligodeoxynucleotides (CpG ODNs): FIX gene therapy construct (99 CpG ODNs in the FIX Padua coding sequence). Blood was collected 4 weeks later and the magnitude of the resulting titer of anti-AAV8 neutralizing antibodies (NAbs) were assayed as a marker of adaptive immune responses to CpG ODNs.

FIG. 7 illustrates FIX consumption by patient for the 12-month period before and after FIX gene therapy construct infusion, in the study described in Example 1.

FIG. 8 illustrates laboratory test results from patient 6 for the period immediately before and after FIX gene therapy construct infusion, in the study described in Example 1.

FIG. 9 illustrates whole exome sequencing variants analysis in patient 5, in the study described in Example 1. Potentially impacting heterozygous and compound heterozygous variants identified uniquely in patient 5.

FIGS. 10A and 10B collectively show the CS04 codon-altered nucleotide sequence (SEQ ID NO: 1) encoding a Factor VIII variant in accordance with some embodiments (“CS04-FL-NA” for full-length coding sequence).

FIGS. 11A and 11B show the CS4m2 codon-altered nucleotide sequence (SEQ ID NO: 2) encoding a Factor VIII variant with the m2 mutants (1105V/A127S/G151K/M166T/L171P (SPI)) amino acid substitutions in accordance with some embodiments (“CS04-FL-NA-m2”).

FIGS. 12A and 12B show the CS04m3 codon-altered nucleotide sequence (SEQ ID NO: 3) encoding a Factor VIII variant with m3 amino acid substitutions in accordance with some embodiments (“CS4-FL-NA-m3”).

FIGS. 13A and 13B show the CS4m23 codon-altered nucleotide sequence (SEQ ID NO: 4) encoding a Factor VIII variant with the m2 mutant set (1105V/A127S/G151K/M66T/L171P (SPI)) and m3 amino acid substitutions in accordance with some embodiments (“CS04-FL-NA-m23”).

FIGS. 14A and 14B show the CS04 m1 codon-altered nucleotide sequence (SEQ ID NO: 5) encoding a Factor VIII variant with an m1 (F328S) amino acid substitution in accordance with some embodiments (“CS04-FL-NA-m1”).

FIGS. 15A and 15B show the CS4m13 codon-altered nucleotide sequence (SEQ ID NO: 6) encoding a Factor VIII variant with m1 and m3 amino acid substitutions in accordance with some embodiments (“CS04-FL-NA-m13”).

FIG. 16 shows the CS06 codon-altered nucleotide sequence (SEQ ID NO: 7) encoding a Factor IX variant with an R384L amino acid substitution (CS06-FL-NA), in accordance with some implementations.

FIG. 17 shows that AAV8-huFIX-null vectors show a higher transduction efficacy and a higher FIX expression. Left panel: Number of vector copies of the gene therapy construct per cell. Right panel: Number of FIXR338L copies/μg RNA in bioreactor cultures treated with AAV8-FIXR338L [6×10¹² vg/kg] or AAV8-FIXR338L CpG-less [6×10¹² vg/kg]. Vector copies per cell were calculated based on 1 μg DNA is equivalent to 150,000 cells.

FIG. 18 shows a normalized time-courses of selected cytokines of two representative donors: Control bioreactors (black circles) and bioreactors treated with AAV8-huFIX-cpg (red squares) or AAV8-huFIX-null (blue triangles). Cytokine expression was overall weak, however, elevated IP-10 and Mip-1a levels were induced by AAV8-huFIX-cpg on days 2-3.

FIG. 19 shows AST and ALT time-courses after cell seeding: Control without infection (circles) and with infection of AAV8-huFIX-cpg (squares) or AAV8-huFIX-null (triangles). Virus-particles were applied for 24 h (day 0-day 1).

FIG. 20 shows that the AAV8-huFIX-cpg vector induced higher anti-AAV8 BABs (left panel) and NABs (right panel) responses than the AAV8-huFIX-null vector, suggesting a stronger activation of the TLR9 pathway by CpGs.

### DETAILED DESCRIPTION OF THE INVENTION

In vivo transgene delivery using viral vectors induces stress response in the target organ, e.g., in the liver. IL-6 signaling is the fundamental stress response pathway against viral pathogens, which might reduce the efficacy of viral-based gene therapy schemes, such as Adenovirus-Associated Virus (AAV)-mediated gene therapy. The present disclosure is based on a genetic finding from a clinical study in 8 hemophilia B patients treated with AAV8-FIX gene therapy. Within the cohort of eight patients, only one patient displayed sustained transgene expression for more than 5 years. In all other patients the expression declined below the therapeutic threshold within 8-12 weeks. In the one patient with sustained transgene expression, a heterozygous missense variant in IL-6 receptor alpha gene was identified. It is therefore postulated that this mutation attenuated the IL-6-mediated stress response in the liver of the patient. Accordingly, among other aspects, the present disclosure provides improved methods for gene therapy, which include concomitant suppression of the IL6-mediated stress response in subject receiving viral vector-based gene therapy.

Advantageously, the improved methods described herein provide a safer and more effective means for improving viral vector-based gene therapy. Previous attempts to attenuate immune responses in patients receiving viral vector-based gene therapy consisted of either on-demand or prophylactic use of non-selective corticosteroids, such as prednisone. Corticosteroids are generally used in the clinic to address potential inflammatory responses including toxicity in the liver. However, the use of high-dose corticosteroids is associated with serious side effects and only sporadic efficacy in rescuing transgene expression upon in vivo AAV-mediated gene delivery. In contrast, anti-IL-6/anti-IL-6R therapies have proven to be safe in the clinic, particularly when used over a limited period of time. Thus, use of anti-IL6/anti-IL6R therapies provides a more specific, focused, and safer suppression of inflammation, facilitating sustained therapeutic transgene expression upon viral vector-based gene therapy, e.g., AAV-mediated gene therapy.

However, in some cases, corticosteroid co-therapy during gene therapy is still desirable because of the beneficial anti-inflammatory properties of corticosteroids. It was found that administration of tocilizumab, an anti-IL6 monoclonal antibody, facilitated corticosteroid sparing, e.g., the ability to lower corticosteroid dose without reducing the efficacy of corticosteroid treatment. Fortunet C. et al., Rheumatology, 54(4):672-77 (2015), the content of which is incorporated herein by reference, in its entirety, for all purposes. Advantageously, as described herein, co-administration of an anti-IL6/IL6R pathway inhibitor and low dose corticosteroid during gene therapy provides the beneficial anti-inflammatory effects of corticosteroid therapy with reduced adverse effects, such as liver damage, fluid retention, bone damage, elevated blood sugars, and problems with mood, memory, and mania.

### Definitions

As used herein and in the appended claims, the singular forms “a.” “an,” and “the” include plural referents unless the context clearly dictates otherwise. Thus, for example, reference to “an antibody” includes a plurality of such antibodies and reference to “a host cell” includes reference to one or more host cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Before the invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

As used herein, “rFIX” refers to recombinant FIX.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

As used herein, “recombinant FIX” includes FIX obtained via recombinant DNA technology. The FIX in the present invention can include all potential forms, including the monomeric and multimeric forms. It should also be understood that the present invention encompasses different forms of FIX to be used in combination. For example, the FIX of the present invention may include different multimers, different derivatives and both biologically active derivatives and derivatives not biologically active.

In the context of the present invention, the recombinant FIX embraces any member of the FIX family from, for example, a mammal such as a primate, human, monkey, rabbit, pig, rodent, mouse, rat, hamster, gerbil, canine, feline, and biologically active derivatives thereof. Mutant and variant FIX proteins having activity are also embraced, as are functional fragments and fusion proteins of the VWF proteins. Furthermore, the FIX of the invention may further comprise tags that facilitate purification, detection, or both. The FIX described herein may further be modified with a therapeutic moiety or a moiety suitable imaging in vitro or in vivo.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. VWF is the predominant species present in a preparation is substantially purified. The term “purified” in some embodiments denotes that a nucleic acid or protein gives rise to essentially one band in an electrophoretic gel. In other embodiments, it means that the nucleic acid or protein is at least 50% pure, more preferably at least 60%, 65%, 70%, 75%, 80%, 85%, 90%, 95%, 96%, 97%, 98%, 99% or more pure. “Purify” or “purification” in other embodiments means removing at least one contaminant from the composition to be purified. In this sense, purification does not require that the purified compound be homogenous, e.g., 100% pure.

As used herein, “administering” (and all grammatical equivalents) includes intravenous administration, intramuscular administration, subcutaneous administration, oral administration, administration as a suppository, topical contact, intraperitoneal, intralesional, or intranasal administration, or the implantation of a slow-release device, e.g., a mini-osmotic pump, to a subject. Administration is by any route including parenteral, and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “therapeutically effective amount or dose” or “therapeutically sufficient amount or dose” or “effective or sufficient amount or dose” refer to a dose that produces therapeutic effects for which it is administered. For example, a therapeutically effective amount of a drug useful for treating hemophilia can be the amount that is capable of preventing or relieving one or more symptoms associated with hemophilia. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the terms “patient” and “subject” are used interchangeably and refer to a mammal (preferably human) that has a disease or has the potential of contracting a disease. The term does not denote a particular age. Thus, both adult and newborn individuals are of interest.

As used herein, the term “about” denotes an approximate range of plus or minus 10% from a specified value. For instance, the language “about 20%” encompasses a range of 18-22%.

As used herein, the term “half-life” refers to the period of time it takes for the amount of a substance undergoing decay (or clearance from a sample or from a patient) to decrease by half.

By “biological sample” is meant a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

By “therapeutically effective dose or amount” is meant an amount that, when administered as described herein, brings about the desired therapeutic response, such as for example, reduced bleeding or shorter clotting times.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Therapeutic antibodies are generally based on the IgG class, which has several subclasses, including, but not limited to IgG1. IgG2. IgG3, and IgG4. In general, IgG1, IgG2 and IgG4 are used more frequently than IgG3.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions.” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3).

A useful comparison of CDR numbering is as below, see Lafranc et al., Dev. Comp. Immunol. 27(1):55-77 (2003):

TABLE 1

| | Kabat + Chothia | IMGT | Kabat | AbM | Chothia | Contact | Xencor |
|---|---|---|---|---|---|---|---|
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly. “CH” domains in the context of IgG are as follows: “CHI” refers to positions 118-220 according to the EU index as in Kabat. “CH2” refers to positions 237-340 according to the EU index as in Kabat, and “CH3” refers to positions 341-447 according to the EU index as in Kabat. As shown herein and described below, the pI variants can be in one or more of the CH regions, as well as the hinge region, discussed below.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CHI domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230. As noted herein, pI variants can be made in the hinge region as well.

As will be appreciated by those in the art, the exact numbering and placement of the heavy constant region domains can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service. National Institutes of Health, Bethesda, entirely incorporated by reference.

TABLE 2

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The light chain generally comprises two domains, the variable light domain (containing the light chain CDRs and together with the variable heavy domains forming the Fv region), and a constant light chain region (often referred to as CL or Cκ).

“Specific binding” or “specifically binds to” or is “specific for” a particular antigen or an epitope, e.g., IL6 or IL6R, means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope, e.g., IL6 or IL6R, can be exhibited, for example, by an antibody having a KD for an antigen or epitope of at least about 10⁻⁴ M, at least about 10⁻⁵ M, at least about 10⁻⁶ M at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope, e.g., IL6 or IL6R, can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a BIACORE® assay.

As used herein, the term “histone deacetylase” or “HDAC” refers to a class of enzymes that have various functions in epigenetic regulation of gene expression. One such function exhibited by some HDACs is the removal of acetyl groups from an ε-N-acetyl lysine amino acid on a histone. HDAC enzymes also deacetylate other targets. However, deacetylation of histones is directly associated with sustained target gene expression. HDAC proteins may be grouped into four classes based on function and DNA sequence similarity. The first two classes are considered “classical” HDACs whose activities are inhibited by trichostatin A (TSA), whereas the third class is a family of NAD+-dependent proteins not affected by TSA and phylogenetically not related to the other three classes. The fourth class is considered an atypical category, based on DNA sequence similarity to the others. Class II is further subdivided into two subclasses: Class IIA and Class IIB, the latter of which is comprised of two independent HDAC domains.

By “bleeding disorder” is meant any disorder associated with excessive bleeding, such as a congenital coagulation disorder, an acquired coagulation disorder, administration of an anticoagulant, or a trauma induced hemorrhagic condition. As discussed below, bleeding disorders may include, but are not limited to, hemophilia A, hemophilia B, von Willebrand disease, idiopathic thrombocytopenia, a deficiency of one or more contact factors, such as Factor XI, Factor XII, prekallikrein, and high molecular weight kininogen (HMWK), a deficiency of one or more factors associated with clinically significant bleeding, such as Factor V, Factor VII, Factor VIII, Factor IX, Factor X. Factor XIII, Factor II (hypoprothrombinemia), and von Willebrand factor, a vitamin K deficiency, a disorder of fibrinogen, including afibrinogenemia, hypofibrinogenemia, and dysfibrinogenemia, an alpha₂-antiplasmin deficiency, and excessive bleeding such as caused by liver disease, renal disease, thrombocytopenia, platelet dysfunction, hematomas, internal hemorrhage, hemarthroses, surgery, trauma, hypothermia, menstruation, and pregnancy.

As used herein, the term “hemophilia” refers to a group of disease states broadly characterized by reduced blood clotting or coagulation. Hemophilia may refer to Type A, Type B, or Type C hemophilia, or to the composite of all three diseases types. Type A hemophilia (hemophilia A) is caused by a reduction or loss of factor VIII (FVIII) activity and is the most prominent of the hemophilia subtypes. Type B hemophilia (hemophilia B) results from the loss or reduction of factor IX (FIX) clotting function. Type C hemophilia (hemophilia C) is a consequence of the loss or reduction in factor XI (FXI) clotting activity. Hemophilia A and B are X-linked diseases, while hemophilia C is autosomal. Conventional treatments for hemophilia include both prophylactic and on-demand administration of clotting factors, such as FVIII, FIX, including Bebulin®-VH, and FXI, as well as FEBA-VH, desmopressin, and plasma infusions.

By “homology” is meant the percent identity between two polypeptide moieties. As referred to herein, two polypeptide sequences are “substantially homologous” to each other when the sequences exhibit about 50% or more sequence identity, such as 60% or more sequence identity, such as 75% or more sequence identity, such as 85% or more sequence identity, such as 90° % or more sequence identity, such as 95% or more sequence identity, including 99% or more sequence identity. In some embodiments, substantially homologous polypeptides include sequences having complete identity to a specified sequence.

By “identity” is meant an exact subunit to subunit correspondence of two polymeric sequences. For example, an identical polypeptide is one that has an exact amino acid-to-amino acid correspondence to another polypeptide or an identical polynucleotide is one that has an exact nucleotide-to-nucleotide correspondence to another polynucleotide. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Any convenient protocol may be employed to determine percent identity between two polymeric sequences, such as for example, ALIGN, Dayhoff, M. O, in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489,1981 for peptide analysis.

The terms “variant,” “analog” and “mutein” refer to biologically active derivatives of a reference molecule, that retain desired activity, such as clotting activity in the treatment of a bleeding disorder. The terms “variant” and “analog” in reference to a polypeptide (e.g., clotting factor) refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. The amino acid sequences of such analogs will have a high degree of sequence homology to the reference sequence, e.g., amino acid sequence homology of 50% or more, such as 60% or more, such as 70% or more, such as 80% or more, such as 90% or more, such as 95% or more, including 99% or more when the two sequences are aligned. In some instances, analogs will include the same number of amino acids but will include substitutions. The term “mutein” further includes polypeptides having one or more amino acid-like molecules including but not limited to compounds contain only amino and/or imino molecules, polypeptides containing one or more analogs of an amino acid (including, for example, synthetic non-naturally occurring amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and 5,977,301; Nguyen et al., Chem. Biol. (2000) 7:463473; and Simon et al., Proc. Natl. Acad. Sci. USA (1992) 89:9367-9371 for descriptions of peptoids). In embodiments of the invention, analogs and muteins have at least the same clotting activity as the native molecule.

Molecular weight, as discussed herein, can be expressed as either a number average molecular weight or a weight average molecular weight. Unless otherwise indicated, all references to molecular weight herein refer to the weight average molecular weight. Both molecular weight determinations, number average and weight average, can be measured using for example, gel permeation chromatography or other liquid chromatography techniques.

As used herein, the terms “Factor VIII” and “FVIII” are used interchangeably, and refer to any protein with Factor VIII activity (e.g., active FVIII, often referred to as FVIIIa) or protein precursor (e.g., pro-protein or pre-pro-protein) of a protein with Factor IXa cofactor activity under particular conditions, e.g., as measured using the two-step chromogenic Factor X activation assay described in Chapter 2.7.4 of the European Pharmacopoeia 9.0. In an exemplary embodiment, a Factor VIII polypeptide refers to a polypeptide that has sequences with high sequence identity (e.g., at least 70%, 75%, 80%, 85%, 90%, 95%, 99%, or more) to the heavy and light chains of a wild type Factor VIII polypeptide. In some embodiments, the B-domain of a Factor VIII polypeptide is deleted, truncated, or replaced with a linker polypeptide to reduce the size of the polynucleotide encoding the Factor VIII polypeptide.

Non-limiting examples of wild type Factor VIII polypeptides include human pre-pro-Factor VIII (e.g., GenBank accession nos. AAA52485, CAA25619, AAA58466, AAA52484, AAA52420, AAV85964, BAF82636, BAG36452, CAI41660, CAI41666, CAI41672, CA143241, CA003404, EAW72645, AAH22513, AAH64380, AAH98389, AAI11968, AAI11970, or AAB61261), corresponding pro-Factor VIII, and natural variants thereof; porcine pre-pro-Factor VIII (e.g., UniProt accession nos. F1RZ36 or K7GSZ5), corresponding pro-Factor VIII, and natural variants thereof: mouse pre-pro-Factor VIII (e.g., GenBank accession nos. AAA37385, CAM15581, CAM26492, or EDL29229), corresponding pro-Factor VIII, and natural variants thereof; rat pre-pro-Factor VIII (e.g., GenBank accession no. AAQ21580), corresponding pro-Factor VIII, and natural variants thereof; rat pre-pro-Factor VIII; and other mammalian Factor VIII homologues (e.g., monkey, ape, hamster, guinea pig, etc.).

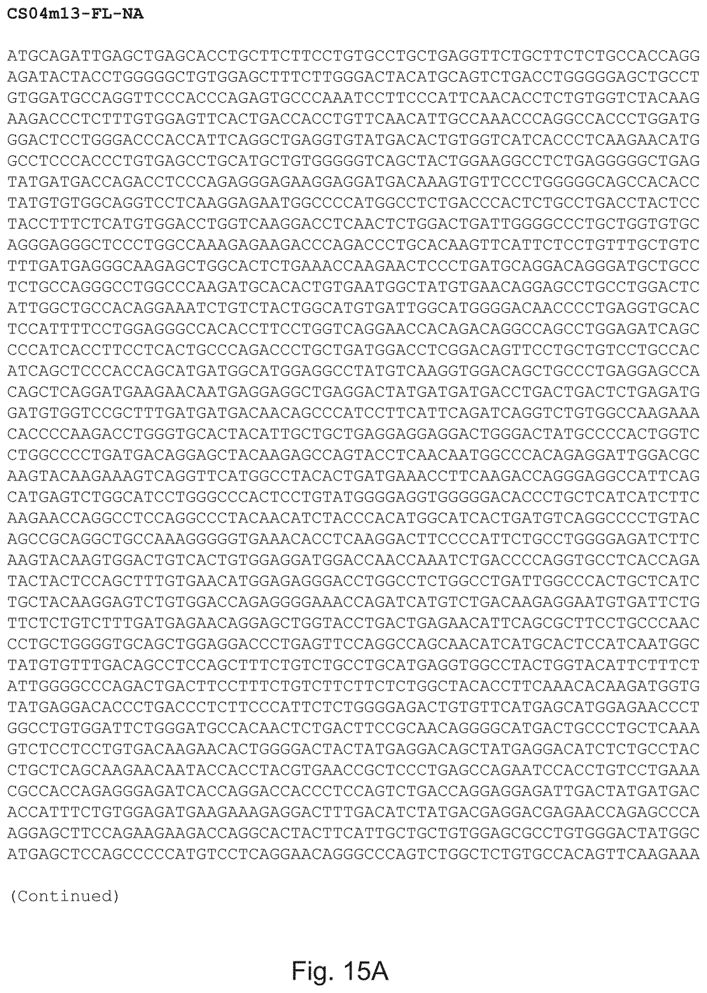

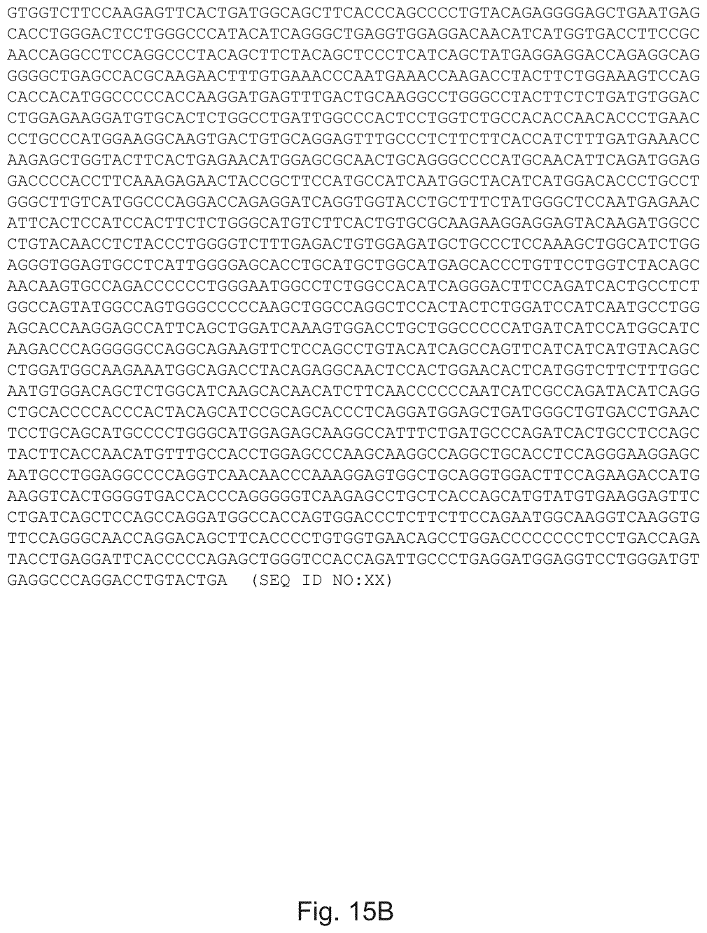

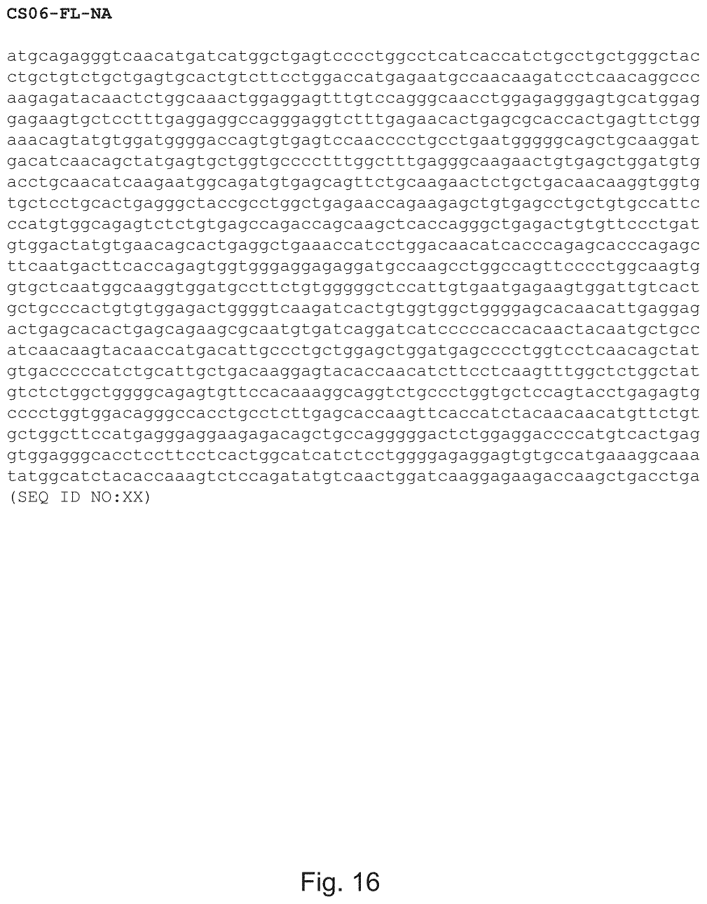

Generally, polynucleotides encoding Factor VIII encode for an inactive single-chain polypeptide (e.g., a pre-pro-protein) that undergoes post-translational processing to form an active Factor VIII protein (e.g., FVIIIa). For example, referring to FIG. 15, the wild type human Factor VIII pre-pro-protein is first cleaved to release the encoded signal peptide (not shown), forming a first single-chain pro-protein (shown as “human wild-type FVIII). The pro-protein is then cleaved between the B and A3 domains to form a first polypeptide that includes the Factor VII heavy chain (e.g., the A1 and A2 domains) and B-domain, and a second polypeptide that includes the Factor VIII light chain (e.g., including the A3, C1, and C3 domains). The first polypeptide is further cleaved to remove the B-domain, and also to separate the A1 and A2 domains, which remain associated with the Factor VIII light chain in the mature Factor VIIIa protein. For review of the Factor VIII maturation process, see Graw et al., Nat Rev Genet., 6(6):488-501 (2005), the content of which is incorporated herein by reference in its entirety for all purposes.

As used herein, the terms “Factor VIII polypeptide” and “FVIII polypeptide” refer to a polypeptide having Factor VIII serine protease activity under particular conditions. Factor VIII polypeptides include single-chain precursor polypeptides (including Factor VIII pre-pro-polypeptides, Factor VIII pro-peptides, and mature, single-chain Factor VIII polypeptides) which, when activated by the post-translational processing described above, become active Factor VIII protein with Factor VIII serine protease activity, as well as the active Factor VIII proteins, themselves. In an exemplary embodiment, a human Factor VIII polypeptide refers to a polypeptide that includes an amino acid sequence with high sequence identity (e.g., at least 85%, 90%, 95%, 99%, or more) to the portion of the wild type human Factor VIII polypeptide that includes the light and heavy chains.

As used herein, a Factor VIII polypeptide includes natural variants and artificial constructs with Factor IX cofactor activity. As used in the present disclosure, Factor VIII encompasses any natural variants, alternative sequences, isoforms, or mutant proteins that retain some basal Factor IX cofactor activity (e.g., at least 5%, 10%, 25%, 50%, 75%, or more of the corresponding wild type activity). Specifically included within the definition of “Factor VIII” are Factor VIII variants, sometimes also referred to as “variant FVIII”. Variant FVIII proteins have at least one amino acid modification as compared to human wild type FVIII. Examples of Factor VIII amino acid variations (relative to FVIII-FL-AA) found in the human population include, without limitation, S19R, R22T, Y24C, Y25C, L26P/R, E30V, W33G, Y35C/H, G41C, R48C/K, K67E/N, L69P, E72K, D75EN/Y, P83R, G89D/V, G92A/V, A97P, E98K, V99D, D101G/H/V, V104D, K108T, M1V, A111T/V, H113R/Y, L117F/R, G121S, E129V, G130R, E132D, Y133C, D135G/Y, T137A/I, S138R, E141K, D145H, V147D, Y155H, V159A, N163K, G164D/V, P165S, C172W, S176P, S179P, V181EM, K185T, D186G/N/Y, S189L, L191F, G193R, L195P, C198G, S202N/R, F214V, L217H, A219D/T, V220G, D222V, E223K, G224W, T252, V253F, N2541, G255V, L261P, P262L, G263S, G266F, C267Y, W274C, H275L, G278R, G280D, E284K, V285G, E291G/K, T2941, F295L, V297A, N299I, R301C/HL, A303E/P, I307S, S308L, F312S, T314A/I, A315V, G323E, L326P, L327PV, C329F, I331V, M339T, E340K, V345A/L, C348R/S/Y, Y365C, R391C/H/P, S392L1P, A394S, W401G, I405F/S, E409G, W412G/R, K427I, L431F/S, R437P/W, I438F, G439D/S/V, Y442C, K444R, Y450D/N, T454I, F455C, G466E, P470L/R/T, G474E/R/V, E475K, G477V, D478N, T479R, F484C, A488G, R490G, Y492C/H, Y492H, I494T, P496R, G498R, R503H, G513S/V, I522Y, K529E, W532G, P540T, T541S, D544N, R546W, R550C/G/H, S553P, S554C/G, V556D, R560T, D561G/H/Y, I567T, P569R, S577F, V578A D579A/H, N583S, Q584H/K/R, I585R-T, M586V, D588G/Y, L594Q, S596P, N601D/K, R602G, S603/R, W604C, Y605H/S, N6091, R612C, N631K/S, M6331, S635N, N637D/I/S, Y639C, L644V, L650F, V653A/M, L659P, A663V, Q664P, F677L, M681I, V682F, Y683C/N, T686R, F698L, M699T/V, M70I, G705V, G710W, N713I, R717L/W, G720D/S, M721I/L, A723T, L725Q, V727F, E739K, Y742C, R795G, P947R, V1012L, E1057K, H1066Y, D1260E, K1289Q, Q1336K, N1460K, L1481P, A1610S, I1698T, Y1699C/F, E1701K, Q1705H, R1708C/H, T1714S, R1715G, A1720V, E1723K, D1727V, Y1728C, R1740G, K1751Q, F1762L, R1768H, G1769R, L1771P, L1775F/N, L1777P, G1779E/R P1780L, I1782R, D1788H, M1791T, A1798P, S1799H, R1800C/G/H, P1801A, Y1802C, S1803Y, F1804S, L1808F, M18421, P1844S, T1845P, E1848G, A1853TV, S1858C, K1864E, D1865N/Y, H1867P/R, G1869D/V, G1872E, P1873R, L1875P, V1876L, C1877R/Y, L1882P, R18881, E1894G, I1901F, E1904D/K, S1907C/R, W1908L, Y1909C, A1939T/V, N1941D/S, G1942A, M1945V, L1951F, R1960L/Q, L1963P, S19651, M19661V, G1967D, S1968R, N1971T, H1973L, G1979V, H1980P/Y, F19821, R1985Q, L1994P, Y1998C, G2000A, T2004R, M2007I, G2013R, W2015C, R2016P/W, E2018G, G2022D, G2028R, S2030N, V2035A, Y2036C, N2038S, 2040Y, G2045EN, I2051S, I2056N, A2058P, W2065R, P2067L, A2070V, S2082N, S2088F, D2093G/Y, H2101D, T2105N, Q2106E/P/R, G2107S, R2109C, I2117F/S, Q2119R, F2120C/L, Y2124C, R2135P, S2138Y, T2141N, M2143V, F2145C, N2148S, N2157D, P2162L, R2169C/H, P2172L/Q/R, T2173A/I, H2174D, R2178CH/L, R2182CH/P, M2183RV, L2185S/W, S21921, C2193G, P2196R, G2198V, E2200D, I2204T, I2209N, A2211P, A2220P, P2224L, R2228G/LP/Q, L2229F, V2242M, W2248C/S, V2251A/E, M2257V, T2264A, Q2265R, F2279C/I, I2281T, D2286G, W2290L, G2304V, D2307A, P2319US, R2323C/G/H/L, R2326G/L/P/Q, Q2330P, W2332R, I2336F, R2339T, G2344C/D/S, and C2345S/Y. Factor VIII proteins also include polypeptides containing post-translational modifications.

As used herein, the terms “Factor VIII polynucleotide” and “FVIII polynucleotide” refer to a polynucleotide encoding a FVIII polypeptide having Factor IXa cofactor activity (e.g., active FVIII, often referred to as FVIIIa) or protein precursor (e.g., pro-protein or pre-pro-protein) of a protein with Factor IXa cofactor activity under particular conditions, e.g., as measured using the two-step chromogenic Factor X activation assay described in Chapter 2.7.4 of the European Pharmacopoeia 9.0.

By “Factor VIII activity” or “Factor IX serine cofactor activity” herein is meant the ability to cleave a Factor X polypeptide in the presence of Factor IXa, e.g., via hydrolysis of the Arg194-Ile195 peptide bond in wild-type Factor IX, thus activating Factor X to Factor Xa. The activity levels can be measured using any Factor VIII activity assay known in the art. One example assay for determining Factor VIII activity is the two-step chromogenic Factor X activation assay described in Chapter 2.7.4 of the European Pharmacopoeia 9.0.

As used herein, the term “FVIII therapy” includes any therapeutic approach of providing factor VIII to a patient to relieve, diminish, or prevent the reoccurrence of one or more symptoms (i.e., clinical factors) associated with hemophilia A. The term encompasses administering any compound, drug, procedure, or regimen comprising factor VIII, including any modified form of factor VIII, which is useful for maintaining or improving the health of an individual with hemophilia and includes any of the therapeutic agents described herein. One skilled in the art will appreciate that either the course of FVIII therapy or the dose of FVIII therapy can be changed, e.g., based upon the results obtained in accordance with the present disclosure.

As used herein, the term “bypass therapy” includes any therapeutic approach of providing non-factor VIII hemostatic agents, compounds or coagulation factors to a patient to relieve, diminish, or prevent the reoccurrence of one or more symptoms (i.e., clinical factors) associated with hemophilia. Non-FVIII compounds and coagulation factors that may be provided include, but are not limited to, Factor VIII Inhibitor Bypass Activity (FEIBA), recombinant activated factor VII (FVIIa), prothrombin complex concentrates, and activated prothrombin complex concentrates. These non-FVIII compounds and coagulation factors may be recombinant or plasma-derived. One skilled in the art will appreciate that either the course of bypass therapy or the dose of bypass therapy can be changed, e.g., based upon the results obtained in accordance with the present invention. The non-FVIII compounds administered in the bypass therapy are referred to herein as “FVIII bypass complex” or “Factor VIII bypass complex.”

As used herein, the terms “Factor IX” and “FIX” (with the “IX” referring to the Roman numerals to mean “nine”) are used interchangeably, and refer to any protein with Factor IX activity (e.g., active FIX, often referred to as “FXa”) or a protein precursor (e.g., a pro-protein or a pre-pro-protein, often referred to as pFIX and ppFIX) of a protein with Factor IX activity, particularly Factor X cleavage activity in the presence of Factor VIII, e.g., as measured using the one stage Factor IX clotting assay described in Chapter 2.7.11 of the European Pharmacopoeia 9.0, the content of which is hereby incorporated by reference.

Non-limiting examples of wild type Factor IX polypeptides include human pre-pro-Factor IX (e.g., GenBank accession nos. NP_000124.1) and NP_001300842.1 (FIX2-FL-AA), corresponding single chain Factor IX lacking the signal peptide (amino acids 1-28 of the pre-pro-protein) and/or propeptide (amino acids 29-46 of the pre-pro-protein), and natural variants thereof; porcine pre-pro-Factor IX (e.g., UniProt accession no. P00741), corresponding single chain Factor IX lacking the signal peptide, and natural variants thereof, murine pre-pro-Factor IX (e.g., UniProt accession no. P16294), corresponding single chain Factor IX lacking the signal peptide, and natural variants thereof; rat pre-pro-Factor IX (e.g., UniProt accession no. P16296), corresponding single chain Factor IX lacking the signal peptide, and natural variants thereof; and other mammalian Factor VIII homologues (e.g., chimpanzee, ape, hamster, guinea pig, etc.).

Factor IX is translated as an inactive, single-chain polypeptide that includes a signal peptide, a propeptide, a light chain, an activation peptide, and a heavy chain, often referred to as a Factor IX pre-pro-polypeptide. The Factor IX pre-pro-peptide undergoes post-translational processing to form an active Factor IX protein (e.g., FIXa). This processing includes removal (e.g., by cleavage) of the signal peptide, followed by removal (e.g., by cleavage) of the propeptide, to form a single-chain mature Factor IX polypeptide, containing the Factor IX light chain and Factor IX heavy chain, which is still inactive. The mature Factor IX polypeptide is further cleaved to excise the activation peptide between the Factor IX light chain and Factor IX heavy chain, forming an active Factor IX protein (e.g., FIXa). The Factor IX light chain and Factor IX light chain remain associated through a disulfide bond. For additional information on the structure, function, and activation of Factor IX see, e.g., Brandstetter H. et al. P.N.A.S. USA, 92(21):9796-800 (1995), Hopfner K P et al., Structure, 7(8):989-96 (1999). Gailani D. et al., Thromb Res., 133 Suppl 1:S48-51 (2014), and U.S. Patent Application Publication No. 2018/0339026, the contents of which are incorporated herein by reference, in their entireties, for all purposes.

As used herein, the terms “Factor IX polypeptide” and “FIX polypeptide” refer to a polypeptide having Factor IX serine protease activity under particular conditions, e.g., as measured using the one stage Factor IX clotting assay described in Chapter 2.7.11 of the European Pharmacopoeia 9.0. Factor IX polypeptides include single-chain precursor polypeptides (including Factor IX pre-pro-polypeptides, Factor IX pro-peptides, and mature, single-chain Factor IX polypeptides) which, when activated by the post-translational processing described above, become active Factor IX protein with Factor IX serine protease activity, as well as the active Factor IX proteins, themselves. Specifically included in the definition of Factor IX polypeptides are Factor IX polypeptides including the R338L variant. In an exemplary embodiment, a human Factor IX polypeptide refers to a polypeptide that includes an amino acid sequence with high sequence identity (e.g., at least 85%, 90%, 95%, 99%, or more) to the portion of the wild type human Factor IX polypeptide that includes the light and heavy chains, FLX-MP-AA or to the portion of the Padua human Factor LX polypeptide that includes the light and heavy chains, FIXp-MP-AA.

As used herein, a Factor IX polypeptide includes natural variants and artificial constructs with Factor X cleavage activity in the presence of Factor VIII. As used in the present disclosure, Factor IX encompasses any natural variants, alternative sequences, isoforms, or mutant proteins that retain some basal Factor IX cleavage activity (e.g., at least 5%, 10%, 25%, 50%, 75%, or more of the corresponding wild type activity as assayed in a one stage clotting assay according to Chapter 2.7.11 of the European Pharmacopoeia 9.0, which is specifically incorporated herein by reference for its teachings of the Assay of Human Coagulation Factor IX in chapter 2.7.11. Examples of Factor IX amino acid variations (relative to FIX-FL-AA) found in the human population include, without limitation, 117N, L20S, C28R, C28Y, V301, R43L, R43Q, R43W, K45N, R46S, R46T, N481, S49P, L52S, E53A, E54D, E54G, F55C, G58A, G58E, G58R, E66V, E67K, F71S, E73K, E73V, R75Q, E79D, T84R, Y91C, D93G, Q96P, C97S, P101R, C102R, C102R, G106D, G106S, C108S, D110N, I112S, N113K, Y115C, C119F, C119R, E124K. G125E, G125R, G125V, C134Y, I136T, N138H, G139D, G139S, C155F, G160E, Q167H, S169C, C170F, C178R. C178W, R191C, R191H, R226G, R226Q, R226W, V227D, V227F, V228F, V228L, Q241H, Q241K. C252S, C252Y, G253E, G253R, A265T, C268W, A279T, N283D, E291V, R294G, R294Q, V296M, H302R, N306S, 1316F, L318R, L321Q, N328K, N328Y, P333H, P333T, T342K, T342M, 1344L, G351D, W356C, G357E, G357R. K362E, G363W, A366D, R379G, R379Q, C382Y, L392F, L383I, R384L, K387E, 1390F, M394K, F395I, F395L, C396F, C396S, A397P, R404T, C407R, C407S, D410H, S411G, S4111, G412E, G413R, P414T. V419E, F424V, T426P, S430T, W431G, W431R. G432S, E433A, G433K. C435Y, A436V, G442E, G442R, 1443T, R449Q, R449W, Y450C, W453R and 1454T. As discussed more fully below, this numbering is relative to the wild type human FIX. Other amino acid variations identified in the human population are known and can be found, for example, using the National Center for Biotechnology Information's (“NCBI”) variation viewer, accession number GCF_000001405.25. Factor VIII proteins also include polypeptides containing post-translational modifications.

Of particular use in the present disclosure is a FIX protein that includes the so called “Padua” mutation, an arginine to leucine change at position 338 of the mature single-strand Factor IX protein (R338L), position 384 of the Factor IX pre-pro-polypeptide (R384L). This mutation confers hyperfunctional activity to the FIX protein. For example, it was shown that “Padua” protein (e.g., Factor IX containing the R338L mutation) is 5-fold to 10-fold more active than wild-type Factor IX in vivo. U.S. Pat. No. 6,531,298; Simioni P. et al., N Engl J Med. 361(17): 1671-75 (2009), hereby incorporated by reference in its entirety. Accordingly, the disclosure provides amino acid and nucleic acid constructs that encode a Padua-FIX protein, sometimes referred to herein as “FIXp” or “pFIX”.

As used herein, the terms “Factor IX polynucleotide” and “FIX polynucleotide” refer to a polynucleotide encoding a Factor IX polypeptide having Factor IX serine protease activity under particular conditions, e.g., as measured using the one stage Factor IX clotting assay described in Chapter 2.7.11 of the European Pharmacopoeia 9.0. Specifically included in the definition of Factor IX polynucleotides are polynucleotides encoding a Factor IX polypeptide that includes the R338L variant.

By “Factor IX activity” or “Factor IX serine protease activity” herein is meant the ability to cleave a Factor X polypeptide in the presence of a Factor VIIIa co-factor, e.g., via hydrolysis of the Arg194-Ile195 peptide bond in wild-type Factor IX, thus activating Factor X to Factor Xa. The activity levels can be measured using any Factor IX activity assay known in the art. An example assay for determining Factor IX activity is the one stage Factor IX clotting assay described in Chapter 2.7.11 of the European Pharmacopoeia 9.0.

As used herein, the term “FIX therapy” includes any therapeutic approach of providing factor IX to a patient to relieve, diminish, or prevent the reoccurrence of one or more symptoms (i.e., clinical factors) associated with hemophilia B. The term encompasses administering any compound, drug, procedure, or regimen comprising factor IX, including any modified form of factor IX, which is useful for maintaining or improving the health of an individual with hemophilia and includes any of the therapeutic agents described herein. One skilled in the art will appreciate that either the course of FIX therapy or the dose of FIX therapy can be changed, e.g., based upon the results obtained in accordance with the present disclosure.

As used herein, the term “vector” refers to any nucleic acid construct used to transfer a nucleic acid encoding a therapeutic protein into a host cell. In some embodiments, a vector includes a replicon, which functions to replicate the nucleic acid construct. Non-limiting examples of vectors useful for gene therapy include plasmids, phages, cosmids, artificial chromosomes, and viruses, which function as autonomous units of replication in vivo. In some embodiments, a vector is a viral vector for introducing a nucleic acid encoding a therapeutic protein into the host cell. Many modified eukaryotic viruses useful for gene therapy are known in the art. For example, adeno-associated viruses (AAVs) are particularly well suited for use in human gene therapy because humans are a natural host for the virus, the native viruses are not known to contribute to any diseases, and the viruses illicit a mild immune response.

As used herein, the term “viral particle” refers to a viral particle encapsulating a polynucleotide encoding a therapeutic protein, which is specific for expression of the therapeutic protein when introduced into a suitable animal host (e.g., a human). Specifically included within the definition of viral particles are recombinant viral particles encapsulating a genome in which a codon-altered polynucleotide, which encodes a therapeutic protein, has been inserted.

As used herein, the term “missense mutation” refers to a change in one amino acid in a protein, arising from a point mutation in a single nucleotide, and is a type of nonsynonymous substitution in a DNA sequence.

As used herein, the term “haplodeficient” describes a genomic state in a diploid genome in which a particular gene includes a mutation rendering one copy of the gene product encoded by the gene non-functional or nearly non-functional, or where one copy of the gene is partially or completely absent from the diploid genome. Accordingly, the term “haploinsufficiency” refers to a state in which the total level and/or activity of a gene product (e.g., a particular protein) is about half of the normal level and/or activity and that reduced activity is not sufficient for normal cellular function. In some embodiments, a haploinsufficiency represents a state where the normal level and/or activity of a gene product is from about 25% to about 75%, or about 300% to about 70%, or about 35% to about 65%, or about 40% to about 60%, or about 45% to about 55% of a wild-type level and/or activity in an organism without a haploinsufficiency. This is true because, in some instances, a cell will compensate for the loss of one functional copy of a particular gene by producing more of the gene product from the other copy of the gene. Similarly, in some instances, a cell will produce less of the gene product from the functional copy of a gene when the other copy of the gene is deleted or rendered non-functional, for example, in cases where a positive feedback loop serves to regulate expression, at least in part, of the gene.

By “AAV” or “adeno-associated virus” herein can refer to a virus derived from a naturally occurring “wild-type” AAV genome into which a polynucleotide encoding a therapeutic protein has been inserted, a recombinant virus derived from a recombinant polynucleotide encoding a therapeutic protein packaged into a capsid using capsid proteins encoded by a naturally occurring AAV cap gene, or a recombinant virus derived from a recombinant polynucleotide encoding a therapeutic protein packaged into a capsid using capsid proteins encoded by a non-natural capsid cap gene. Included within the definition of AAV are serotypes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), and AAV type 9 (AAV9) viruses encapsulating a polynucleotide encoding a therapeutic protein and viruses formed by one or more variant AAV capsid proteins encapsulating a polynucleotide encoding a therapeutic protein.

As used herein, the term “gene therapy” includes any therapeutic approach of providing a nucleic acid encoding a therapeutic protein to a patient to relieve, diminish, or prevent the reoccurrence of one or more symptoms (e.g., clinical factors) associated with a disorder. The term encompasses administering any compound, drug, procedure, or regimen comprising a nucleic acid encoding a therapeutic protein, including any modified form of the therapeutic protein, for maintaining or improving the health of an individual with a disorder, e.g., a deficiency in the activity of the endogenous therapeutic protein. One skilled in the art will appreciate that either the course of gene therapy or the dose of a gene therapy therapeutic agent can be changed, e.g., based upon the results obtained in accordance with the present disclosure.

As used herein, the term “gene” refers to the segment of a DNA molecule that codes for a polypeptide chain (e.g., the coding region). In some embodiments, a gene is positioned by regions immediately preceding, following, and/or intervening the coding region that are involved in producing the polypeptide chain (e.g., regulatory elements such as a promoter, enhancer, polyadenylation sequence, 5′-untranslated region, 3′-untranslated region, or intron).

As used herein, the term “regulatory elements” refers to nucleotide sequences, such as promoters, enhancers, terminators, polyadenylation sequences, introns, etc., that provide for the expression of a coding sequence in a cell.

As used herein, the term “promoter element” refers to a nucleotide sequence that assists with controlling expression of a coding sequence. Generally, promoter elements are located 5′ of the translation start site of a gene. However, in certain embodiments, a promoter element may be located within an intron sequence, or 3′ of the coding sequence. In some embodiments, a promoter useful for a gene therapy vector is derived from the native gene of the target protein. In some embodiments, a promoter useful for a gene therapy vector is specific for expression in a particular cell or tissue of the target organism (e.g., a liver-specific promoter). In yet other embodiments, one of a plurality of well characterized promoter elements is used in a gene therapy vector described herein. Non-limiting examples of well-characterized promoter elements include the CMV early promoter, the 0-actin promoter, and the methyl CpG binding protein 2 (MeCP2) promoter. In some embodiments, the promoter is a constitutive promoter, which drives substantially constant expression of the target protein. In other embodiments, the promoter is an inducible promoter, which drives expression of the target protein in response to a particular stimulus (e.g., exposure to a particular treatment or agent). For a review of designing promoters for AAV-mediated gene therapy, see Gray et al. (Human Gene Therapy 22:1143-53 (2011)), the contents of which are expressly incorporated by reference in their entirety for all purposes.

As used herein, the term “CpG” refers to a cytosine-guanine dinucleotide along a single strand of DNA, with the “p” representing the phosphate linkage between the two.

As used herein, the term “CpG island” refers to a region within a polynucleotide having a statistically elevated density of CpG dinucleotides. As used herein, a region of a polynucleotide (e.g., a polynucleotide encoding a therapeutic protein) is a CpG island if, over a 200-base pair window: (i) the region has GC content of greater than 50%, and (ii) the ratio of observed CpG dinucleotides per expected CpG dinucleotides is at least 0.6, as defined by the relationship:

$$\frac{N [ CpG ] \times N [ length of window ]}{N [ C ] \times N [ G ]} \geq 0.6$$

For additional information on methods for identifying CpG islands, see Gardiner-Garden M. et al., J Mol Biol., 196(2):261-82 (1987), the content of which is expressly incorporated herein by reference, in its entirety, for all purposes.

As used herein, the term “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). However, particularly useful embodiments herein, for use in gene therapy in patients, use phosphodiester bonds.

The term “amino acid” refers to naturally occurring and non-natural amino acids, including amino acid analogs and amino acid mimetics that function in a manner similar to the naturally occurring amino acids. Naturally occurring amino acids include those encoded by the genetic code, as well as those amino acids that are later modified, e.g., hydroxyproline, y-carboxyglutamate, and O-phosphoserine. Naturally occurring amino acids can include, e.g., D- and L-amino acids. As to amino acid sequences, one of ordinary skill in the art will recognize that individual substitutions, deletions or additions to a nucleic acid or peptide sequence that alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

As used herein, the term “liver-specific expression” refers to the preferential or predominant in vivo expression of a particular gene in hepatic tissue, as compared to in other tissues. In some embodiments, liver-specific expression means that at least 50% of all expression of the particular gene occurs within hepatic tissues of a subject. In other embodiments, liver-specific expression means that at least 55%, 60%, 65%, 70%, 75%, 80%, 85%, 90%, 95%, 99%, or 100% of all expression of the particular gene occurs within hepatic tissues of a subject. Accordingly, a liver-specific regulatory element is a regulatory element that drives liver-specific expression of a gene in hepatic tissue.

As described herein, polynucleotides encoding therapeutic proteins can include regulatory elements, such as promoters, enhancers, terminators, polyadenylation sequences, and introns, as well viral packaging elements, such as inverted terminal repeats (“ITRs”), and/or other elements that support replication of the polynucleotide in a non-viral host cell, e.g., a replicon supporting propagation of the polynucleotide, e.g., in a bacterial, yeast, or mammalian host cell.

Of particular use in the present disclosure are codon-altered polynucleotides encoding therapeutic proteins. As described herein, the codon-altered polynucleotides provide increased expression of the transgenic therapeutic protein in vivo, as compared to the level of the therapeutic protein expression provided by a natively-coded construct (e.g., a polynucleotide encoding the same therapeutic amino acid sequence using the wild-type human codons). As used herein, the term “increased expression” refers to an increased level of the transgenic therapeutic protein in the blood of an animal administered the codon-altered polynucleotide, as compared to the level of the transgenic therapeutic protein in the blood of an animal administered a natively-coded construct. Increased expression of the protein leads to an increase in the protein's activity: thus, increased expression leads to increased activity.

In some embodiments, increased expression refers to at least 25% greater transgenic therapeutic polypeptide in the blood of an animal administered the codon-altered polynucleotide, as compared to the level of the transgenic therapeutic polypeptide in the blood of an animal administered a natively-coded polynucleotide. For the purpose of the present disclosure, increased expression refers to an effect generated by the alteration of the codon sequence, rather than hyperactivity caused by an underlying amino acid substitution, e.g., a “Padua” mutation in Factor IX. That is, the expression level obtained from a codon-optimized sequence encoding a “Padua” Factor IX polynucleotide is compared relative to the expression level obtained from a natively-coded “Padua” protein. In some embodiments, increased expression refers to at least 50% greater, at least 75% greater, at least 100% greater, at least 3-fold greater, at least 4-fold greater, at least 5-fold greater, at least 6-fold greater, at least 7-fold greater, at least 8-fold greater, at least 9-fold greater, at least 10-fold greater, at least 15-fold greater, at least 20-fold greater, at least 25-fold greater, at least 30-fold greater, at least 40-fold greater, at least 50-fold greater, at least 60-fold greater, at least 70-fold greater, at least 80-fold greater, at least 90-fold greater, at least 100-fold greater, at least 125-fold greater, at least 150-fold greater, at least 175-fold greater, at least 200-fold greater, at least 225-fold greater, or at least 250-fold greater transgenic therapeutic polypeptide in the blood of an animal administered the codon-altered polynucleotide, as compared to the level of transgenic therapeutic polypeptide in the blood of an animal administered a natively coded polynucleotide. Therapeutic polypeptide levels in the blood of an animal can be measured, for example, using an ELISA assay specific for the therapeutic polypeptide.

IL-6 is a cytokine also termed B cell stimulating factor-2 (BSF2) or interferon β2. IL-6 was discovered as a differentiation factor involved in activation of B lymphocyte lineage cells (Hirano, T. et al., Nature, 324:73-76, 1986), and after then it has been demonstrated that IL-6 is the multifunctional cytokine which affects functions of various cells (Akira, S. et al., Adv, in Immunology, 54:1-78, 1993). It has been reported that IL-6 induces maturation of T lymphocyte lineage cells (Lotz. M. et al., J. Exp. Med., 167:1253-1258, 1988).

IL-6 transmits its biological activity via two types of protein on cells. One is IL-6 receptor (also referred to herein as IL-6R) which is a ligand binding protein with molecular weight of about 80 kD, to which IL-6 binds (Taga, T. et al., J. Exp. Med., 166:967-981, 1987; Yamasaki, K. et al., Science, 241:825-828, 1987). IL-6 receptor also occurs as soluble IL-6 receptor mainly composed of its extracellular region, in addition to a membrane binding type which penetrates through cell membrane and expresses on the cell membrane.

As referred to herein, “silencing mediator of retinoid or thyroid hormone receptor-2/nuclear receptor co-repressor 2 gene” or “SMRT/NCOR-2 gene” refers to the gene encoding the silencing mediator of retinoid or thyroid hormone receptor-2/nuclear receptor co-repressor 2, a nuclear receptor corepressor required for formation of a nuclear receptor co-repressor complex that mediates transcriptional silencing of target genes via recruitment of general chromatin modifying enzymes such as histone deacetylases and methyltransferases. The SMRT complex directly interacts with HNF4α, thereby potentially directly affecting HNF4α-mediated TTR promoter expression.

As used herein, the term “genotype” refers to the genetic makeup of an individual. The terms “genotyping” or “genotypic assay” refer to a process of determining the genotype of an individual with a biological assay. The biological assays used for genotyping include or a combination of techniques such as, for example, polymerase chain reaction (PCR), DNA fragment analysis, allele specific oligonucleotide (ASO) probes, DNA sequencing, DNA microarrays, etc. Common genotyping techniques include, but are not limited to, restriction fragment length polymorphism (RFLP), terminal restriction fragment length polymorphism (t-RFLP), amplified fragment length polymorphism (AFLP), multiplex ligation-dependent probe amplification (MLPA), and whole exome sequencing and variant analysis.

In an embodiment, the genotyping assay includes whole exome sequencing and variant analysis. For example, in an embodiment, genomic DNA of the patient is extracted from whole blood samples treated with EDTA. Exome enrichment is then performed using commercially available kits. A fragmented DNA library is constructed for the sample and high-throughput sequencing is performed. The sequence reads are aligned using, for example, Burrows-Wheeler Aligner. Variations in the whole exome of the patient (when compared to whole genome of other like individuals) are evaluated using Combined Annotation Dependent Depletion (CADD) to score likely deleteriousness of single nucleotide variants and insertions/deletions in the human genome to determine whether the patient has mutations in the genes of interest (e.g., those are associated with increased sensitivity to persistent infection by a viral-based gene therapy vector).

### INTRODUCTION

In clinical trials for treating patients with hemophilia B with AAV based FIX gene therapy, transaminitis and the instability of FIX expression were found to be associated with vector dose-dependent production of circulating cytotoxic T cells (CTLs) recognizing the AAV capsid, leading to the hypothesis that adaptive CTL IRs might be responsible for the clearance of transduced hepatocytes and the loss of FIX expression.

This effect had not previously been observed in preclinical animal studies with AAV FIX gene therapy, and the proposed model of CTL-mediated transgene loss has left several unanswered questions. Firstly, evidence suggests that AAV capsid antigen presentation is most efficient immediately after AAV administration and then declines, implying that capsid-specific CTLs should eliminate most AAV-transduced hepatocytes at earlier time-points. There are also difficulties explaining how prednisolone can restore FIX activity after CTL-mediated removal of transduced hepatocytes. Both single-stranded and self-complementary (scAAV) vectors were demonstrated to upregulate innate immune signaling early after vector infusion, through Toll-like receptor 9 (TLR9; one of the critical virus sensors involved in recognition of pathogen-associated molecular patterns [PAMPS]), and a role for the innate immune system in orchestrating later responses to long-term AAV-transduction has also been proposed.

The vector stocks used in an earlier AAV FIX gene therapy trial contained an excess of AAV empty capsids (only an estimated ˜15% contained vector genomes), so that the remaining majority of capsids were potentially immunogenic but did not carry FIX genes. If an AAV capsid dose-dependent T cell response was the sole mechanism underlying the transaminitis observed, then greatly reducing the AAV empty capsid load could potentially avoid the complication of transaminitis and associated loss of FIX activity.

Subsequent efforts to reduce the immunogenicity of AAV vectors used initially for FIX gene therapy have focused on maximizing FIX expression, while reducing exposure to the viral vector. Switching from single-stranded DNA to scAAV vectors (demonstrated to be more potent in preclinical studies), as well as codon optimization of the FIX sequence, have facilitated the use of lower vector doses. In addition, the AAV serotype 8 (AAV8; a rhesus macaque serotype) was used in preference to AAV2 because of the lower reported seroprevalence of neutralizing antibodies (NAbs) in humans. AAV8 is also associated with improved tropism towards hepatocytes, helping to lower vector doses while permitting delivery into a peripheral vein.

The scAAV8 FIX expression strategy has also been re-designed to incorporate a hyperactive FIX variant (FIX Padua). This naturally occurring single amino acid variant contains a gain-of-function mutation (leucine substituted for arginine in position 338) that results in a 5- to 10-fold increase in specific activity relative to the wild-type FIX protein.

The development of the AAV8-based FIX Padua gene therapy FIX gene therapy construct incorporated the hyperactive FIX Padua variant to maximize FIX expression, and included purification steps to reduce the amount of AAV empty capsid to 30% to minimize the potential for immune system activation. Encouraging results in pre-clinical testing led to the further development of FIX gene therapy construct as a potential treatment for hemophilia B designed to improve FIX activity to levels expected to provide effective protection from joint bleeding.

However, persistent expression of the transgene remains an issue. Advantageously, the present disclosure provides methods for improved gene therapy through improved persistent expression of the transgene. Specifically, in some embodiments, the improved persistent expression is achieved by concomitant suppression of the IL6 signaling pathway and/or the NCoR2/SMRT deacetylation pathway.

The present disclosure also provides a method for identifying patients for viral-based gene therapy, not only for hemophilia but for any other disease that can potentially be treated with gene therapy, and a method for treating a patient with a viral-based gene therapy that promote persistent expression of the gene therapy vector after administration.

Methods for Improved Viral-Vector Based Gene Therapy

As described in Example 1, out of eight hemophilia B patients administered an AAV-based Factor IX gene therapy vector, only a single patient (patient 5) maintained expression of transgenic Factor IX from the gene therapy vector for over four years. Genomic analysis of patient 5 revealed that the patient had mutations in their interleukin-6 (IL6) and NCOR2/SMRT genes. Accordingly, in one aspect, the present disclosure provides methods for improved gene therapy that simulate the suppressed IL6 and/or NCOR2/SMRT function observed in this patient, by concomitant administration of an inhibitor of the interleukin-6 (IL6) signaling pathway or the NCOR2/SMRT histone deacetylation pathway, and a viral-based gene therapy vector.

### Co-Administration of IL6 Pathway Inhibitors

Accordingly, in some embodiments, the methods described herein include administration of an inhibitor of the interleukin-6 (IL6) signaling pathway. In some embodiments, the inhibitor is an IL6 inhibitor. For instance, in some embodiments, the IL6 inhibitor is a monoclonal antibody or a derivative thereof, e.g., an IL6-specific binding molecule engineered based on the CDR sequences of an anti-IL6 monoclonal antibody. Several anti-IL6 monoclonal antibodies are approved for therapeutic use or are in pre-clinical/clinical trials. These antibodies include siltuximab, olokizumab, elsilimomab, clazakizumab, sirukumab, gerilimzumab, FM101, and MEDI5117. Non-limiting examples of anti-IL6 monoclonal antibodies, variants thereof, and derivatives thereof are described, for example, in U.S. Pat. Nos. 7,291,721, 7,560,112, 7,612,182, 7,820,155, 7,919,095, 7,955,597, 8,062,866, 8,632,774, 9,234,034, U.S. Patent Application Publication Nos. 2014/0127209, 2011/0059080, and PCT Publication No. WO 2019/109947, the contents of which are hereby incorporated herein by reference, in their entireties, for all purposes.

Accordingly, in some embodiments, the method includes administering to the patient a therapeutically effective dose of a viral-based gene therapy vector and siltuximab. In related embodiments, the method includes administering to the patient a therapeutically effective dose of a viral-based gene therapy vector and a variant of siltuximab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of siltuximab, e.g., a derivative of an antibody containing one or more of the CDR regions of siltuximab. For more information on siltuximab, which is marketed under the tradename SYLVANT®, see U.S. Pat. Nos. 7,612,182 and 7,291,721, the contents of which are hereby incorporated herein by reference, in their entireties, for all purposes.

Similarly, in some embodiments, the method includes administering to the patient a therapeutically effective dose of a viral-based gene therapy vector and olokizumab. In related embodiments, the method includes administering to the patient a therapeutically effective dose of a viral-based gene therapy vector and a variant of olokizumab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of olokizumab, e.g., a derivative of an antibody containing one or more of the CDR regions of olokizumab.

Similarly, in some embodiments, the method includes administering to the patient a therapeutically effective dose of a viral-based gene therapy vector and elsilimomab. In related embodiments, the method includes administering to the patient a therapeutically effective dose of a viral-based gene therapy vector and a variant of elsilimomab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of elsilimomab, e.g., a derivative of an antibody containing one or more of the CDR regions of elsilimomab.

Similarly, in some embodiments, the method includes administering to the patient a therapeutically effective dose of a viral-based gene therapy vector and clazakizumab. In related embodiments, the method includes administering to the patient a therapeutically effective dose of a viral-based gene therapy vector and a variant of clazakizumab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of clazakizumab, e.g., a derivative of an antibody containing one or more of the CDR regions of clazakizumab.

Similarly, in some embodiments, the method includes administering to the patient a therapeutically effective dose of a viral-based gene therapy vector and sirukumab. In related embodiments, the method includes administering to the patient a therapeutically effective dose of a viral-based gene therapy vector and a variant of sirukumab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of sirukumab, e.g., a derivative of an antibody containing one or more of the CDR regions of sirukumab. For more information on sirukumab, see U.S. Pat. No. 7,560,112, the content of which is hereby incorporated herein by reference, in its entirety, for all purposes.

Similarly, in some embodiments, the method includes administering to the patient a therapeutically effective dose of a viral-based gene therapy vector and gerilimzumab. In related embodiments, the method includes administering to the patient a therapeutically effective dose of a viral-based gene therapy vector and a variant of gerilimzumab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of gerilimzumab, e.g., a derivative of an antibody containing one or more of the CDR regions of gerilimzumab.

Similarly, in some embodiments, the methods described herein include administration of an inhibitor of the interleukin-6 receptor (IL6R). In some embodiments, the IL6R inhibitor is a monoclonal antibody or a derivative thereof, e.g., an IL6R-specific binding molecule engineered based on the CDR sequences of an anti-IL6R monoclonal antibody. Several anti-IL6 monoclonal antibodies are approved for therapeutic use or are in pre-clinical/clinical trials. These antibodies include tocilizumab, sarilumab, levilimab, vobarilizumab, or satralizumab. Non-limiting examples of anti-IL6 monoclonal antibodies, variants thereof, and derivatives thereof are described, for example, in U.S. Pat. Nos. 5,795,965, 7,582,298, 8,337,849, 8,562,991, 9,017,678, 9,173,880, 9,828,430, and 10,618,964, U.S. Patent Application Publication No. 2017/0166646, and PCT Publication Nos. WO 2018/029182, WO 2018/112237, and WO 2019/052457, the contents of which are hereby incorporated herein by reference, in their entireties, for all purposes.

Accordingly, in some embodiments, the method includes administering to the patient a therapeutically effective dose of a viral-based gene therapy vector and tocilizumab. In related embodiments, the method includes administering to the patient a therapeutically effective dose of a viral-based gene therapy vector and a variant of tocilizumab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of tocilizumab, e.g., a derivative of an antibody containing one or more of the CDR regions of tocilizumab. For more information on siltuximab, which is marketed under the tradename ACTEMRA®, see U.S. Pat. No. 5,795,965, the content of which is hereby incorporated herein by reference, in its entirety, for all purposes.

Similarly, in some embodiments, the method includes administering to the patient a therapeutically effective dose of a viral-based gene therapy vector and sarilumab. In related embodiments, the method includes administering to the patient a therapeutically effective dose of a viral-based gene therapy vector and a variant of sarilumab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of sarilumab, e.g., a derivative of an antibody containing one or more of the CDR regions of sarilumab. For more information on sarilumab, which is marketed under the tradename KEVZARA®, see U.S. Pat. No. 7,582,298, the content of which is hereby incorporated herein by reference, in its entirety, for all purposes.

Similarly, in some embodiments, the method includes administering to the patient a therapeutically effective dose of a viral-based gene therapy vector and levilimab. In related embodiments, the method includes administering to the patient a therapeutically effective dose of a viral-based gene therapy vector and a variant of levilimab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of levilimab, e.g., a derivative of an antibody containing one or more of the CDR regions of levilimab.

Similarly, in some embodiments, the method includes administering to the patient a therapeutically effective dose of a viral-based gene therapy vector and vobarilizumab. In related embodiments, the method includes administering to the patient a therapeutically effective dose of a viral-based gene therapy vector and a variant of vobarilizumab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of vobarilizumab, e.g., a derivative of an antibody containing one or more of the CDR regions of vobarilizumab.

Similarly, in some embodiments, the method includes administering to the patient a therapeutically effective dose of a viral-based gene therapy vector and satralizumab. In related embodiments, the method includes administering to the patient a therapeutically effective dose of a viral-based gene therapy vector and a variant of satralizumab, e.g., a monoclonal antibody with one or more amino acid substitutions in the constant region of the antibody, one or more amino acid substitutions in the variable region of the antibody, and/or one or more amino acid substitutions in a CDR of the antibody. Similarly, in some embodiments, the method includes administering to the patient a therapeutically effective dose of a viral-based gene therapy vector and a derivative of satralizumab, e.g., a derivative of an antibody containing one or more of the CDR regions of satralizumab. For more information on satralizumab, see U.S. Pat. No. 8,562,991, the content of which is hereby incorporated herein by reference, in its entirety, for all purposes.

Interleukin 6 (IL-6) is an interleukin that acts as both a pro-inflammatory cytokine and an anti-inflammatory myokine. The overproduction of IL-6 is implicated in the pathogenesis of a variety of diseases, including several chronic inflammatory diseases and cancer. IL6 is known to act within the JAK/STAT signaling pathway, the Ras/MAK signaling pathway, the SHP-2/ERK MAPK signaling pathway, and the PI3K/Akt signaling pathway. In the liver, the IL6/IL6R signal transduction system functions to activate two intercellular signaling pathways, the SHP-2/ERK MAPK pathway and the JAK/STAT pathway. See, Mihara M. et al., Clin Sci (Lond), 122(4):143-59 (2012), the content of which is hereby incorporated by reference, in its entirety, for all purposes. Accordingly, in some embodiments, the methods described herein include administration of an inhibitor of a factor, e.g., other than IL6 or IL6R, in one of these pathways.

In some embodiments, the methods described herein include administration of an inhibitor of a factor in the JAK/STAT signaling pathway in concert with a viral-based gene therapy vector. For instance, in some embodiments, the methods include administration of a JAK inhibitor. JAK inhibitors function by inhibiting the activity of one or more of the Janus kinase family of enzymes (JAK1, JAK2, JAK3, TYK2), thereby interfering with the JAK-STAT signaling pathway. Non-limiting examples of JAK inhibitors include ruxolitinib, tofacitinib, oclacitinib, baricitinib, peficitinib, fedratinib, upadacitinib, filgotinib, cerdulatinib, gandotinib, lestaurtinib, momelotinib, pacritinib, abrocitinib, cucurbitacin 1, and CHZ868. Similarly, in some embodiments, the methods described herein include administration of a STAT inhibitor. STAT inhibitors function by inhibiting the activity of one or more of the signal transducer and activator of transcription factors (STAT1, STAT2, STAT3, STAT4, STAT5A, STAT5B, STAT6). Non-limiting examples of STAT inhibitors include pioglitazone, methotrexate, sirolimus, tacrolimus, sirolimus, AT9283, crytotanshinone, capsaicin, curcumin, curcubitacin 1, celastrol, atriprimod, and sulforaphane. For more information on JAK and STAT inhibitors in clinical trials, see Chin-Yap L. et al., Front. Oncol., 9(48) (2019), the content of which is incorporated herein by reference, in its entirety, for all purposes.

Similarly, in some embodiment, the methods described herein include administration of an inhibitor of a factor in the SHP-2/ERK MAPK signaling pathway in concert with a viral-based gene therapy vector. Non-limiting examples of inhibitors of the MAPK signaling pathway include somatostatin analogs (e.g., SOM230 and octreotide), dopamine and agonists thereof (e.g., dopamine, bromocriptine, and cabergoline), TGF-beta 18-beta-glycyrrhetinic acid, BIM-23A760, usolic acid, and fulvestrant. For more information on inhibitors of the MAPK signaling pathway, see Lu M et al., Frontiers in Endocrinology, 10(330) (2019).

### Co-administration of Histone Deacetylase Inhibitors

In some embodiments, the methods described herein include administration of an inhibitor of the NCOR2/SMRT histone deacetylation pathway. The NCOR2/SMRT gene encodes a nuclear receptor co-repressor that mediates transcriptional silencing of certain target genes. The encoded protein is a member of a family of thyroid hormone- and retinoic acid receptor-associated co-repressors. This protein acts as part of a multi-subunit complex which includes histone deacetylases to modify chromatin structure that prevents basal transcriptional activity of target genes. Aberrant expression of this gene is associated with certain cancers. Alternate splicing results in multiple transcript variants encoding different isoforms.

Accordingly, in some embodiments, the methods described herein provide an improved method for gene therapy that includes administering to the patient a therapeutically effective dose of a viral-based gene therapy vector and a histone deacetylase inhibitor.

Several histone deacetylase inhibitors have been identified that act on class I. IIa and IIb HDACs, typically by binding to the zinc-containing catalytic domain of the HDACs. These inhibitors fall into several groupings, including hydroxamic acids (e.g., tricostatin A), cyclic tetrapeptides (e.g., trapoxin B) and depsipeptides, benzamides, electrophilic ketones, and aliphatic acid compounds (e.g., phenylbutyrate and valproic acid). Second-generation inhibitors derived from these groupings include the hydroxamic acids vorinostat (SAHA), belinostat (PXD101), LAQ824, panobinostat (LBH-589), givinostat (ITF2357), and the benzamides entinostat (MS275). CL-994, and mocetinostat (MGCD0103). Generally, benzamides inhibit class I, but not class II HDACs, and hydroxamic acids are stronger inhibitors of class I and IIb HDACs than of class IIa HDACs, while isoform selectivity within a class is significantly lower.

### Therapeutic Proteins

Generally, the methods described herein are suitable for use with a viral-based gene therapy vector, e.g., an adeno-associated gene therapy vector, encoding any therapeutic protein, since the proposed mechanism of action is independent of the protein being expressed from the vector. Examples of the types of therapeutic proteins that are well-suited for use in the methods described herein include blood coagulation factors, serine proteases, cytokines, soluble portions of cytokine receptor proteins, immunoglobulins, soluble portions of a T-cell receptor, soluble portions of a major histocompatibility complex (MHC) protein, complement regulatory proteins, growth factors, soluble portions of hormone receptor proteins, soluble portions of cholesterol receptor proteins, transcription factor proteins, and metabolic enzymes.

In fact, there are several gene therapies that have been awarded regulatory approval. For example, talimogene laherparepvec (encoding granulocyte-macrophage colony-stimulating factor (GM-CSF)), voretigene neparvovec-rzyl (encoding retinoid isomerohydrolase (RPE65)), and onasemnogene abeparvovec-xioi (encoding survival of motor neuron 1 (SMN1)). Accordingly, in some embodiments, the methods described herein include administration of an IL6/IL6R inhibitor and a viral-based gene therapy vector encoding a granulocyte-macrophage colony-stimulating factor (GM-CSF) polypeptide, a retinoid isomerohydrolase (RPE65) polypeptide, or a survival of motor neuron 1 (SMN1) polypeptide. In specific embodiments, the methods described herein include administration of an IL6/IL6R inhibitor and one of talimogene laherparepvec, voretigene neparvovec-rzyl, and onasemnogene abeparvovec-xioi.

In some embodiments, the gene therapy vector encodes a blood factor, e.g., a coagulation factor, e.g., Factor II, Factor V, Factor VII, Factor VIII, Factor IX, Factor X, Factor XI, or Factor XII. Accordingly, in some embodiments, the methods described herein include administration of an IL6/IL6R inhibitor and a viral-based gene therapy vector encoding Factor II, Factor V. Factor VII, Factor VIII, Factor IX, Factor X, Factor XI, or Factor XII.

### Factor VIII

In some embodiments, the patient has hemophilia A and the viral-based gene therapy vector encodes a Factor VIII polypeptide. In an embodiment, the encoded Factor VIII polypeptide is a B-domain deleted Factor VIII polypeptide. In some embodiments, the present disclosure provides codon-altered polynucleotides encoding Factor VIII variants. These codon-altered polynucleotides provide markedly improved Factor VIII biopotency (e.g., activity) when administered in an AAV-based gene therapy construct. The codon-altered polynucleotides also demonstrate improved AAV-virion packaging, as compared to conventionally codon-optimized constructs.

Wild-type Factor VIII is encoded with a 19 amino acid signal peptide, which is cleaved from the encoded polypeptide prior to activation of Factor VIII. As appreciated by those in the art the Factor VIII signal peptide may be mutated, replaced by signal peptides from other genes or Factor VIII genes from other organisms, or completely removed, without affecting the sequence of the mature polypeptide left after the signal peptide is removed by cellular processing.

Accordingly, in some embodiments, a codon-altered polynucleotide (e.g., a nucleic acid composition) provided herein has a nucleotide sequence with high sequence identity to the portions encoding a Factor VIII heavy and light chains, and a short, 14 amino acid, B-domain substituted linker (e.g., the “SQ” linker containing a furin cleavage site to facilitate maturation of an active FVIIIa protein in vivo), that further includes one or more of the five “X5 mutations” (e.g., one, two, three, four, or all five of the 1105V/A127S/G151K/M166T/L171P mutations (SPI numbering: (SPE numbering is 186V/A108S/G132K/M147T/L152P, respectively)), relative to the full-length human wild type Factor VIII sequence), and/or a short glycosylation peptide inserted into the B-domain substituted linker (e.g., an SQ linker).

Specifically, the X5 mutation set is based on the fact that substitution of porcine amino acids 82-176 for the corresponding human amino acids in a B-domain deleted gene therapy construct increased Factor VIII activity when expressed in HEK293 cells (W. Xiao, communication). Back-mutation of single porcine amino acids into the human BDD-FVIII construct identified five amino acids within the A1 domain that contribute to this phenomenon: 1105V, A127S, G151K, M166T, and L171P (SPI). Introduction of the combination of these mutations into the human construct recapitulated the improved activity of the larger porcine substitution. Accordingly, in some embodiments, the encoded Factor VIII polypeptides include one or more amino acid substitutions selected from 1105V, A127S, G151K. M166T, and L171P, with the entire 5 amino acid set finding particular use in many embodiments.

In some embodiments, a Factor VIII polypeptide includes a mutation within an 11 amino acid hydrophobic β-sheet in the A1 domain, which interacts with BiP, increase secretion of Factor VII. For example, an F328S (SPI F309S SPE) amino acid substitution within the pocket increased Factor VIII secretion 3-fold. The number of the variants can be done inclusive of the signal peptide, “Signal Peptide Inclusive”, or “SPI”, or starting from the processed final protein sequence, “Signal Peptide Exclusive”, or “SPE”. Thus, using SPI numbering, the mutation F328S is the same as the F309 SPE mutant. Generally the specification uses the SPI numbering, but as will be appreciated by those in the art, either numbering system results in the same mutation(s).

Other Factor VIII variants are known to provide advantageous properties. For example, mutation of residues A108, R121, and L2302 (SPE), located at the interface between the A1 and C2 domains, increases the stability of Factor VIII. For example, the A1081 amino acid substitution introduces a hydrophobic residue that better fills the inter-domain space, stabilizing the interaction. Likewise, an R121C/L2302C (SPE) double amino acid substitution introduces a disulfide bond spanning the A1-C2 domains, further stabilizing the interaction. Taken together, all three amino acid substitutions increase the thermal stability of Factor VIII by 3 to 4-fold. For review, see Wakabayashi et al., J Biol Chem. 286(29):25748-55 (2011) and Wakabayashi et al., Thromb Haemost. 10(3):492-95 (2012).

Similarly, mutation of E113 (SPE), located within the calcium binding domain of Factor VIII, increases the specific FVIII clotting activity. For example, E113A appears to increase FXase formation through increased FVIII affinity for Factor IXa. Specifically, the E113A amino acid substitution increases specific FVIII clotting activity two-fold and increases affinity for Factor IXa by four-fold (Biochemistry, 41:8485 (2002); J. Biol. Chem., 279:12677 (2004); and Biochemistry, 44:10298 (2005)).

Substitution of one or more amino acid residues surrounding the Factor VIII APC cleavage site (residues 331-341 (SPE)) reduce Factor VIIIa inactivation by activated protein C, without affecting FVIII activity. For example PQL333-335VDQ (SPE) amino acid substitutions reduce Factor VIII inactivation by 16-fold. Likewise, MKN336-339GNQ amino acid substitutions reduce Factor VIII inactivation by 9-fold. When combined, the two triple amino acid substitutions (e.g., PQLRMKN333-339VDQRGNQ) (SEQ ID NOs: 8 and 9, respectively) reduce Factor VIII inactivation by 100-fold (J. Biol. Chem., 282:20264 (2007). Accordingly, in some embodiments, the encoded Factor VIII polypeptide include PQL333-335VDQ and/or MKN337-339GNQ (SPE) amino acid substitutions.

Mutations within the A2 domain interface also increase Factor VIII stability. Specifically, mutating charged residues in the A1-A2 and A2-A3 domain interfaces increases stability and retention of the A2 subunit in Factor VIIIa. For example, mutation of D519, E665, and E1984 to V or A yields up to 2-fold increased stability in Factor VIII and up to 5-fold stability in Factor VIIIa. Specifically, D519A/E665V amino acid substitutions provide a 3-fold increase in stability: D519V/E665V amino acid substitutions provide a 2-fold increase in stability, an 8-fold decrease in A2 dissociation, and a 2-4-fold increase in thrombin generation potential; D519V/E1984A amino acid substitutions provide a 2-fold increase in stability; and D519V/E665V/E1984A amino acid substitution provide a 2-fold increase in stability (Blood 112:2761-69 (2008); J. Thromb. Haemost., 7:438-44 (2009)).

Substitution of seven amino acids for six across the HC-B domain interface that introduces an additional glycosylation site introduced close to the interface. Accordingly, in some embodiments, m3 is the deletion of amino acids AIEPRSF755-761 (SEQ ID NO: 10) and the insertion of amino acids TTYVNRSL (SEQ ID NO: 11) after N754, relative to FVIII-FL-AA (e.g., AIEPRSF755-761TTYVNRSL) (“TTYVNRSL” disclosed as SEQ ID NO: 11). Residues AIEPR755-759, relative to the wild type Factor VIII sequence, fall within the end of the heavy chain, while residues S760 and F761 fall within the B-domain. In some embodiments, where the FVIII B-domain is deleted, truncated, or replaced, residues S760 and F761 may not be present in the underlying amino acid sequence being mutated.

In additional embodiments, the polypeptides and polynucleotides of the disclosure include m4 mutations. Elimination of the C1899-C1903 disulfide bond in Factor VIII also increased secretion. Moreover, the increases in Factor VIII secretion are additive for the combination of F328S (SPI, F309S SPE) and C1918G/C1922G amino acid substitutions (Miao et al., Blood, 103:3412-19 (2004); Selvaraj et al., J. Thromb. Haemost., 10:107-15 (2012)).

In some embodiments, the linkage between the FVIII heavy chain and the light chain (e.g., the B-domain in wild-type Factor VIII) is further altered. Due to size constraints of AAV packaging capacity, B-domain deleted, truncated, and or linker substituted variants should improve the efficacy of the FVIII gene therapy construct. The most conventionally used B-domain substituted linker is that of SQ FVIII, which retains only 14 amino acids of the B domain as linker sequence. Another variant of porcine VIII (“OBI-1,” described in U.S. Pat. No. 6,458,563) is well expressed in CHO cells, and has a slightly longer linker of 24 amino acids. In some embodiments, the Factor VIII constructs encoded by the codon-altered polynucleotides described herein include an SQ-type B-domain linker sequence. In other embodiments, the Factor VIII constructs encoded by the codon-altered polynucleotides described herein include an OBI-1-type B-domain linker sequence.

In some embodiments, the encoded Factor VIII polypeptides described herein include an SQ-type B-domain linker, including amino acids 760-762/1657-1667 of the wild-type human Factor VIII B-domain (Sandberg et al. Thromb. Haemost. 85:93 (2001)). In some embodiments, the SQ-type B-domain linker has one amino acid substitution relative to the corresponding wild-type sequence. In some embodiments, the SQ-type B-domain linker has two amino acid substitutions relative to the corresponding wild-type sequence. In some embodiments, a glycosylation peptide is inserted into the SQ-type B-domain linker.

In some embodiments, the encoded Factor VIII polypeptides described herein include a Greengene-type B-domain linker, including amino acids 760/1582-1667 of the wild-type human Factor VIII B-domain (Oh et al., Biotechnol. Prog., 17:1999 (2001)). In some embodiments, the Greengene-type B-domain linker has one amino acid substitution relative to the corresponding wild-type sequence. In some embodiments, the Greengene-type B-domain linker has two amino acid substitutions relative to the corresponding wild-type sequence. In some embodiments, a glycosylation peptide is inserted into the Greengene-type B-domain linker.

In some embodiments, the encoded Factor VIII polypeptides described herein include an extended SQ-type B-domain linker (SFSQNPPVLKRHQR (SEQ ID NO: 12)), including amino acids 760-769/1657-1667 of the wild-type human Factor VIII B-domain (Tim et al., Haemophilia, 16:349 (2010)). In some embodiments, the extended SQ-type B-domain linker has one amino acid substitution relative to the corresponding wild-type sequence. In some embodiments, the extended SQ-type B-domain linker has two amino acid substitutions relative to the corresponding wild-type sequence. In some embodiments, a glycosylation peptide is inserted into the extended SQ-type B-domain linker.

In some embodiments, the encoded Factor VIII polypeptides described herein include a porcine OBI-1-type B-domain linker, including the amino acids SFAQNSRPPSASAPKPPVLRRHQR (SEQ ID NO: 13) from the wild-type porcine Factor VIII B-domain (Toschi et al., Curr. Opin. Mol. Ther. 12:517 (2010)). In some embodiments, the porcine OBI-1-type B-domain linker has one amino acid substitution relative to the corresponding wild-type sequence. In some embodiments, the porcine OBI-1-type B-domain linker has two amino acid substitutions relative to the corresponding wild-type sequence. In some embodiments, a glycosylation peptide is inserted into the porcine OBI-1-type B-domain linker.

In some embodiments, the encoded Factor VIII polypeptides described herein include a human OBI-1-type B-domain linker, including amino acids 760-772/1655-1667 of the wild-type human Factor VIII B-domain (FVIII-FL-AA). In some embodiments, the human OBI-1-type B-domain linker has one amino acid substitution relative to the corresponding wild-type sequence. In some embodiments, the human OBI-1-type B-domain linker has two amino acid substitutions relative to the corresponding wild-type sequence. In some embodiments, a glycosylation peptide is inserted into the human OBI-1-type B-domain linker.

In some embodiments, the encoded Factor VIII polypeptides described herein include an 08-type B-domain linker, including the amino acids SFSQNSRHQAYRYRRG (SEQ ID NO: 14) from the wild-type porcine Factor VIII B-domain (Toschi et al., Curr. Opin. Mol. Ther. 12:517 (2010)). In some embodiments, the porcine OBI-1-type B-domain linker has one amino acid substitution relative to the corresponding wild-type sequence. In some embodiments, the porcine OBI-1-type B-domain linker has two amino acid substitutions relative to the corresponding wild-type sequence. In some embodiments, a glycosylation peptide is inserted into the porcine OBI-1-type B-domain linker.

Removal of the B-domain from Factor VIII constructs does not appear to affect the activity of the activated enzyme (e.g., FVIIIa), presumably because the B-domain is removed during activation. However, the B-domain of Factor VIII contains several residues that are post-translationally modified. e.g., by N- or O-linked glycosylation. In silico analysis (Prediction of N-glycosylation sites in human proteins, R. Gupta, E. Jung and S. Brunak, in preparation (2004)) of the wild-type Factor VIII B-domain predicts that at least four of these sites are glycosylated in vivo. It is thought that these modifications within the B-domain contribute to the post-translational regulation and/or half-life of Factor VIII in vivo.

While the Factor VIII B-domain is absent in mature Factor VIIIa protein, glycosylation within the B-domain of the precursor Factor VIII molecule may increase the circulating half-life of the protein prior to activation. Thus, in some embodiments, the polypeptide linker of the encoded Factor VIII constructs described herein includes one or more glycosylation sequences, to allow for glycosylation in vivo. In some embodiments, the polypeptide linker includes at least one consensus glycosylation sequence (e.g., an N- or O-linked glycosylation consensus sequence). In some embodiments, the polypeptide linker includes at least two consensus glycosylation sequences. In some embodiments, the polypeptide linker includes at least three consensus glycosylation sequences. In some embodiments, the polypeptide linker includes at least four consensus glycosylation sequences. In some embodiments, the polypeptide linker includes at least five consensus glycosylation sequences. In some embodiments, the polypeptide linker includes at least 6, 7, 8, 9, 10, or more consensus glycosylation sequences.

In some embodiments, the polypeptide linker contains at least one N-linked glycosylation sequence N—X-ST, where X is any amino acid other than P, S, or T. In some embodiments, the polypeptide linker contains at least two N-linked glycosylation sequences N—X-S/T, where X is any amino acid other than P. S, or T. In some embodiments, the polypeptide linker contains at least three N-linked glycosylation sequences N—X-S/T, where X is any amino acid other than P, S, or T. In some embodiments, the polypeptide linker contains at least four N-linked glycosylation sequences N—X-S/T, where X is any amino acid other than P, S, or T. In some embodiments, the polypeptide linker contains at least five N-linked glycosylation sequences N—X-S/T, where X is any amino acid other than P, S, or T. In some embodiments, the polypeptide linker contains at least 6, 7, 8, 9, 10, or more N-linked glycosylation sequences N—X-S/T, where X is any amino acid other than P, S, or T.

### Factor VIII

In some embodiments, the patient has hemophilia A and the viral-based gene therapy vector encodes a Factor VIX polypeptide. In an embodiment, the encoded Factor IX polypeptide has an R338L amino acid change relative to the wild type Factor IX sequence. In some embodiments, the present disclosure provides codon-altered polynucleotides encoding Factor IX variants. These codon-altered polynucleotides provide markedly improved Factor IX biopotency (e.g., activity) when administered in an AAV-based gene therapy construct. The codon-altered polynucleotides also demonstrate improved AAV-virion packaging, as compared to conventionally codon-optimized constructs.

### Co-Administration of Corticosteroids

In some embodiments, the methods described above for improved gene therapy also include administering, to the human patient, a course of a corticosteroid (e.g., prednisolone or prednisone) in concert with an inhibitor of the IL6/IL6R signaling pathway and/or NCoR2/SMRT deacetylation pathway, e.g., to reduce the level of an inflammatory response, for example, by lowering the subject's production of cytokines and/or chemokines. Example methods for co-administering prednisolone or prednisone with a gene therapy are described, for example, in International Patent Application Publication No. WO 2008/069942, the content of which is incorporated herein by reference, in its entirety, for all purposes.

In some embodiments, the corticosteroid (e.g., prednisolone or prednisone) is administered to the human patient prior to administering the viral-based gene therapy vector (e.g., adeno-associated virus (AAV) particles). For example, in some embodiments, the corticosteroid (e.g., prednisolone or prednisone) is administered about a week, or about one or two days, before the viral-based gene therapy vector (e.g., AAV particles) are administered to the patient. In some embodiments, a course of the corticosteroid (e.g., prednisolone or prednisone) is administered starting about a week, or about one or two days, before the viral-based gene therapy vector (e.g., AAV particles) are administered, and is continued after administration of the viral-based gene therapy vector (e.g., AAV particles).

In some embodiments, the corticosteroid (e.g., prednisolone or prednisone) is co-administered to the human subject when administering the viral-based gene therapy vector (e.g., adeno-associated virus (AAV) particles). For example, in some embodiments, the corticosteroid (e.g., prednisolone or prednisone) is administered on the same day, e.g., directly before or after administration of the viral-based gene therapy vector (e.g., AAV particles). In some embodiments, a course of the corticosteroid (e.g., prednisolone or prednisone) is administered on the same day as the viral-based gene therapy vector (e.g., AAV particles) are administered, and is continued after administration of the viral-based gene therapy vector (e.g., AAV particles).

In some embodiments, the corticosteroid (e.g., prednisolone or prednisone) is administered to the patient after administering the viral-based gene therapy vector (e.g., adeno-associated virus (AAV) particles). For example, in some embodiments the corticosteroid (e.g., prednisolone or prednisone) is first administered about one or two days after the viral-based gene therapy vector (e.g., AAV particles) are administered to the patient.

It should be noted that the corticosteroid (e.g., prednisolone or prednisone) is a small molecule drug that is administered orally (although it can also be administered intravenously), and thus “co-administration” in this context does not require that a single solution contains both drugs.

In some embodiments, the course of the corticosteroid (e.g., prednisolone or prednisone) is administered to the patient over a period of at least two weeks, e.g., daily or every two days. In some embodiments, the course of the corticosteroid (e.g., prednisolone or prednisone) is administered over a period of at least three weeks. In some embodiments, the dose of the corticosteroid (e.g., prednisolone or prednisone) decreases during the course. For example, in one embodiment, the course begins with administration of about 60 mg of the corticosteroid (e.g., prednisolone or prednisone) per day, and is reduced as the course progresses.

In one embodiment, the course includes administration of about 60 mg of the corticosteroid (e.g., prednisolone or prednisone) per day to the human patient, during the first week of the course, administration of about 40 mg of the corticosteroid (e.g., prednisolone or prednisone) per day to the patient, during the second week of the course, and administration of about 30 mg of the corticosteroid (e.g., prednisolone or prednisone) per day to the patient, during the third week immediately following infusion of the AAV particles.

In some embodiments, the course includes further tapering administration of the corticosteroid (e.g., prednisolone or prednisone) after the third week, e.g., administration of a tapering dose of the corticosteroid (e.g., prednisolone or prednisone). In one embodiment, the tapering dose of the corticosteroid (e.g., prednisolone or prednisone) includes successively administering doses (e.g., one or more doses at each concentration) of about 20 mg the corticosteroid (e.g., prednisolone or prednisone) per day, about 15 mg the corticosteroid (e.g., prednisolone or prednisone) per day, about 10 mg the corticosteroid (e.g., prednisolone or prednisone) per day, and about 5 mg the corticosteroid (e.g., prednisolone or prednisone) per day.

In one embodiment, the tapering dose of the corticosteroid (e.g., prednisolone or prednisone) includes administration of about 20 mg of the corticosteroid (e.g., prednisolone or prednisone) per day to the patient, for 5 consecutive days (e.g., immediately) following completion of the initial course of the corticosteroid (e.g., prednisolone or prednisone), administration of about 15 mg of the corticosteroid (e.g., prednisolone or prednisone) per day to the patient, for 3 consecutive days (e.g., immediately) following the 5 days on which the patient was administered 20 mg of the corticosteroid (e.g., prednisolone or prednisone), administration of about 10 mg of the corticosteroid (e.g., prednisolone or prednisone) per day to the patient, for 3 consecutive days (e.g., immediately) following the 3 days on which the patient was administered 15 mg of the corticosteroid (e.g., prednisolone or prednisone), and administration of about 5 mg of the corticosteroid (e.g., prednisolone or prednisone) per day to the patient, for 3 consecutive days (e.g., immediately) following the 3 days on which the patient was administered 10 mg of the corticosteroid (e.g., prednisolone or prednisone).

In one embodiment, the tapering dose of the corticosteroid (e.g., prednisolone or prednisone) includes administration of about 30 mg of the corticosteroid (e.g., prednisolone or prednisone) per day to the patient, for 7 consecutive days immediately following completion of the initial course of the corticosteroid (e.g., prednisolone or prednisone), administration of about 20 mg of the corticosteroid (e.g., prednisolone or prednisone) per day to the patient, for 7 consecutive days immediately following the 7 days on which the patient was administered 30 mg of the corticosteroid (e.g., prednisolone or prednisone), administration of about 15 mg of the corticosteroid (e.g., prednisolone or prednisone) per day to the patient, for 5 consecutive days immediately following the 7 days on which the human subject was administered 20 mg of the corticosteroid (e.g., prednisolone or prednisone), administration of about 10 mg of the corticosteroid (e.g., prednisolone or prednisone) per day to the patient, for 5 consecutive days immediately following the 5 days on which the patient was administered 15 mg of the corticosteroid (e.g., prednisolone or prednisone), and administration of about 5 mg of the corticosteroid (e.g., prednisolone or prednisone) per day to the patient, for 5 consecutive days immediately following the 5 days on which the patient was administered 10 mg of the corticosteroid (e.g., prednisolone or prednisone).

In some embodiments, the length of a tapering dose of the corticosteroid (e.g., prednisolone or prednisone) administered to the patient is determined based on whether the patient is still exhibiting signs of liver inflammation at the end of the initial course of the corticosteroid (e.g., prednisolone or prednisone), e.g., as indicated by a reduction in expression of the transgene, a reduction in the expression in the activity of the expressed therapeutic protein, or increases in liver enzymes.

In some embodiments, an increase in the level of liver enzymes in the patient indicates liver inflammation in the subject. For example, in some embodiments, the level of liver enzymes in the patient is monitored following administration of the viral-based gene therapy vector (e.g., AAV particles), and the patient is administered a course of prednisolone or prednisone if an increase in the level of liver enzymes (e.g., more than a threshold increase in the amount of liver enzymes, e.g., as compared to a baseline level of liver enzymes in the patient before administration of the vector or shortly after administration of the vector) is detected. For more details on various regimens for co-administering a corticosteroid with a viral-based gene therapy vector, see PCT Application PCT/US19/41802, filed on Jul. 19, 2019, the content of which is incorporated herein by reference, in its entirety, for all purposes.

Given the clinical evidence for corticosteroid sparing by tocilizumab, in some embodiments, the methods described herein include administration of an anti-IL6IL6R pathway inhibitor and a low dose corticosteroid regimine. In some embodiments, low-dose corticosteroid therapy includes a dose of no more than 30 mg of the corticosteroid (e.g., prednisolone or prednisone) per day. In some embodiments, low-dose corticosteroid therapy includes a dose of no more than 25 mg of the corticosteroid (e.g., prednisolone or prednisone) per day. In some embodiments, low-dose corticosteroid therapy includes a dose of no more than 20 mg of the corticosteroid (e.g., prednisolone or prednisone) per day. In some embodiments, low-dose corticosteroid therapy includes a dose of no more than 15 mg of the corticosteroid (e.g., prednisolone or prednisone) per day. In some embodiments, low-dose corticosteroid therapy includes a dose of no more than 10 mg of the corticosteroid (e.g., prednisolone or prednisone) per day. In some embodiments, low-dose corticosteroid therapy includes a dose of no more than 7.5 mg of the corticosteroid (e.g., prednisolone or prednisone) per day. In some embodiments, low-dose corticosteroid therapy includes a dose of no more than 5 mg of the corticosteroid (e.g., prednisolone or prednisone) per day. In some embodiments, low-dose corticosteroid therapy includes a dose of no more than 2.5 mg of the corticosteroid (e.g., prednisolone or prednisone) per day. In some embodiments, low-dose corticosteroid therapy includes a dose of from 1 mg per day to 30 mg per day of the corticosteroid (e.g., prednisolone or prednisone). In some embodiments, low-dose corticosteroid therapy includes a dose of from mg per day to 20 mg per day of the corticosteroid (e.g., prednisolone or prednisone).

In some embodiments, low-dose corticosteroid therapy includes a dose of from 1 mg per day to 15 mg per day of the corticosteroid (e.g., prednisolone or prednisone). In some embodiments, low-dose corticosteroid therapy includes a dose of from 1 mg per day to 10 mg per day of the corticosteroid (e.g., prednisolone or prednisone). In some embodiments, low-dose corticosteroid therapy includes a dose of from 1 mg per day to 7.5 mg per day of the corticosteroid (e.g., prednisolone or prednisone). In some embodiments, low-dose corticosteroid therapy includes a dose of from 1 mg per day to 5 mg per day of the corticosteroid (e.g., prednisolone or prednisone). In some embodiments, low-dose corticosteroid therapy includes a dose of from 1 mg per day to 30 mg per day of the corticosteroid (e.g., prednisolone or prednisone). In some embodiments, low-dose corticosteroid therapy includes a dose of about 1 mg, 2.5 mg, 5 mg, 7.5 mg, 10 mg, 12.5 mg, 15 mg, 20 mg, 25 mg, or 30 mg per day of the corticosteroid (e.g., prednisolone or prednisone).

Similarly as described above, in some embodiments, the methods described herein include an initial course of low dose corticosteroid, when administered with an IL6/IL6R poathway inhibitor, followed by a reduction in dose and/or tapering of the dose. In some embodiments, a further reduced dose is administered for an extended time period. However, in some embodiments, the further reduced dose is not administered daily but, rather, every other day, every third day, twice weekly, weekly, bi-weekly, monthly, quarterly, semi-annually, annually, and the like. In some embodiments, a low-dose corticosteroid is administered in an on-demand fashion.

### Methods for Identifying Suitable Patients

In one aspect, the present disclosure relates to a method for treating a patient with viral-based gene therapy that promotes or ensures persistent expression of the gene therapy vector after administration. The method includes determining whether the patient has a genotype sensitizing the patient to persistent infection by a viral-based gene therapy vector by one or both of: (i) evaluating whether the patient has a mutation in the SMRT/NCOR2 gene associated with reduced SMRT/NCOR2 protein function, and (ii) evaluating whether the patient has a mutation in the interleukin-6 receptor (IL-6R) gene associated with reduced IL-6R function. If the patient has either a mutation in the SMRT/NCOR2 gene associated with reduced SMRT/NCOR2 protein function or a mutation in the IL-6R gene associated with reduced IL-6R function, a viral-based gene therapy vector is assigned to the patient.

In an embodiment, determining whether the patient has a genotype sensitizing the patient to persistent infection by a viral-based gene therapy vector includes determining whether the patient has a mutation in the SMRT/NCOR2 or IL-6R genes associated with increased sensitivity to persistent infection by a viral-based gene therapy vector by, for example, obtaining a biological sample from the patient, and performing a genotypying assay to on the biological sample to determine whether the patient has a mutation in the SMRT/NCOR2 or IL-6R genes.

In some embodiments, the genotype sensitizing the patient to persistent infection by a viral-based gene therapy vector includes mutations in both copies of the patient's SMRT/NCOR2 gene that reduce the protein function of the encoded SMRT/NCOR2 proteins by at least 75% relative to the wild type SMRT/NCOR2 protein function.

In some embodiments, the genotype sensitizing the patient to persistent infection by a viral-based gene therapy vector includes a mutation in at least one copy of the patient's IL-6R gene that causes IL-6R haplodeficiency. In an embodiment, the mutation in the at least one copy of the patient's IL-6R gene is a missense mutation in the IL-6R gene.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure, a method for treating a patient with a viral-based gene therapy includes determining whether the patient has a genotype sensitizing the patient to persistent infection by a viral-based gene therapy vector by evaluating whether the patient has a mutation in the interleukin-6 receptor (IL-6R) gene associated with reduced IL-6R function. A viral-based gene therapy vector is administered to the patient if the patient has a mutation in the IL-6R gene associated with reduced IL-6R function.

In some embodiments, determining whether the patient has a genotype sensitizing the patient to persistent infection by a viral-based gene therapy vector includes determining whether the patient has a mutation in the IL-6R gene associated with increased sensitivity to persistent infection by a viral-based gene therapy vector by, for example, obtaining a biological sample from the patient, and performing a genotypying assay to on the biological sample to determine whether the patient has a mutation in the IL-6R gene.

In some embodiments, the genotype sensitizing the patient to persistent infection by a viral-based gene therapy vector includes a mutation in at least one copy of the patient's IL-6R gene that causes IL-6R haplodeficiency. In an embodiment, the mutation in the at least one copy of the patient's IL-6R gene is a missense mutation in the IL-6R gene.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure, a method for treating a patient with a viral-based gene therapy includes determining whether the patient has a genotype sensitizing the patient to persistent infection by a viral-based gene therapy vector by evaluating whether the patient has a mutation in the SMRT/NCOR2 gene associated with reduced SMRT/NCOR2 protein function. A viral-based gene therapy vector is administered to the patient if the patient has a mutation in the SMRT/NCOR2 gene associated with reduced SMRT/NCOR2 protein function.

In some embodiments, determining whether the patient has a genotype sensitizing the patient to persistent infection by a viral-based gene therapy vector includes determining whether the patient has a mutation in the SMRT/NCOR2 gene associated with increased sensitivity to persistent infection by a viral-based gene therapy vector by, for example, obtaining a biological sample from the patient, and performing a genotypying assay to on the biological sample to determine whether the patient has a mutation in the SMRT/NCOR2 gene.

In some embodiments, the genotype sensitizing the patient to persistent infection by a viral-based gene therapy vector includes mutations in both copies of the patient's SMRT/NCOR2 gene that reduce the protein function of the encoded SMRT/NCOR2 proteins by at least 75% relative to the wild type SMRT/NCOR2 protein function.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the protein therapeutic includes Factor VIII. In an embodiment, the protein therapeutic includes a Factor VIII bypass complex. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the protein therapeutic includes Factor IX. In an embodiment, the encoded Factor IX polypeptide has an R338L amino acid change relative to the wild type Factor LX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure a method for treating a disease associated with insufficient level of an enzymatic activity in a patient includes determining whether the patient has a genotype sensitizing the patient to persistent infection by a viral-based gene therapy vector by one or both of: (i) evaluating whether the patient has a mutation in the SMRT/NCOR2 gene associated with reduced SMRT/NCOR2 protein function, and (ii) evaluating whether the patient has a mutation in the interleukin-6 receptor (IL-6R) gene associated with reduced IL-6R function. A viral-based gene therapy vector is administered to the patient if the patient has either a mutation in the SMRT/NCOR2 gene associated with reduced SMRT/NCOR2 protein function or a mutation in the IL-6R gene associated with reduced IL-6R function. A protein therapeutic having the enzymatic activity is administered to the patient if the patient does not have either or both a mutation in the SMRT/NCOR2 gene associated with reduced SMRT/NCOR2 protein function or a mutation in the IL-6R gene associated with reduced IL-6R function.

In an embodiment, determining whether the patient has a genotype sensitizing the patient to persistent infection by a viral-based gene therapy vector includes determining whether the patient has a mutation in the SMRT/NCOR2 or IL-6R genes associated with increased sensitivity to persistent infection by a viral-based gene therapy vector by, for example, obtaining a biological sample from the patient, and performing a genotypying assay to on the biological sample to determine whether the patient has a mutation in the SMRT/NCOR2 or IL-6R genes.

In some embodiments, the genotype sensitizing the patient to persistent infection by a viral-based gene therapy vector includes mutations in both copies of the patient's SMRT/NCOR2 gene that reduce the protein function of the encoded SMRT/NCOR2 proteins by at least 75% relative to the wild type SMRT/NCOR2 protein function.

In some embodiments, the genotype sensitizing the patient to persistent infection by a viral-based gene therapy vector includes a mutation in at least one copy of the patient's IL-6R gene that causes IL-6R haplodeficiency. In an embodiment, the mutation in the at least one copy of the patient's IL-6R gene is a missense mutation in the IL-6R gene.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the protein therapeutic includes Factor VIII. In an embodiment, the protein therapeutic includes a Factor VIII bypass complex. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the protein therapeutic includes Factor IX. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure a method for treating a disease associated with insufficient level of an enzymatic activity in a patient includes determining whether the patient has a genotype sensitizing the patient to persistent infection by a viral-based gene therapy vector by evaluating whether the patient has a mutation in the interleukin-6 receptor (IL-6R) gene associated with reduced IL-6R function. A viral-based gene therapy vector is administered to the patient if the patient has either a mutation in the IL-6R gene associated with reduced IL-6R function. A protein therapeutic having the enzymatic activity is administered to the patient if the patient does not have a mutation in the IL-6R gene associated with reduced IL-6R function.

In an embodiment, determining whether the patient has a genotype sensitizing the patient to persistent infection by a viral-based gene therapy vector includes determining whether the patient has a mutation in the IL-6R gene associated with increased sensitivity to persistent infection by a viral-based gene therapy vector by, for example, obtaining a biological sample from the patient, and performing a genotypying assay to on the biological sample to determine whether the patient has a mutation in the IL-6R gene.

In some embodiments, the genotype sensitizing the patient to persistent infection by a viral-based gene therapy vector includes a mutation in at least one copy of the patient's IL-6R gene that causes IL-6R haplodeficiency. In an embodiment, the mutation in the at least one copy of the patient's IL-6R gene is a missense mutation in the IL-6R gene.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the protein therapeutic includes Factor VIII. In an embodiment, the protein therapeutic includes a Factor VIII bypass complex. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the protein therapeutic includes Factor IX. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure a method for treating a disease associated with insufficient level of an enzymatic activity in a patient includes determining whether the patient has a genotype sensitizing the patient to persistent infection by a viral-based gene therapy vector by evaluating whether the patient has a mutation in the SMRT/NCOR2 gene associated with reduced SMRT/NCOR2 protein function. A viral-based gene therapy vector is administered to the patient if the patient has either a mutation in the SMRT/NCOR2 gene associated with reduced SMRT/NCOR2 protein function. A protein therapeutic having the enzymatic activity is administered to the patient if the patient does not have a mutation in the SMRT/NCOR2 gene associated with reduced SMRT/NCOR2 protein function.

In an embodiment, determining whether the patient has a genotype sensitizing the patient to persistent infection by a viral-based gene therapy vector includes determining whether the patient has a mutation in the SMRT/NCOR2 gene associated with increased sensitivity to persistent infection by a viral-based gene therapy vector by, for example, obtaining a biological sample from the patient, and performing a genotypying assay to on the biological sample to determine whether the patient has a mutation in the SMRT/NCOR2 gene.

In some embodiments, the genotype sensitizing the patient to persistent infection by a viral-based gene therapy vector includes mutations in both copies of the patient's SMRT/NCOR2 gene that reduce the protein function of the encoded SMRT/NCOR2 proteins by at least 75% relative to the wild type SMRT/NCOR2 protein function.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the protein therapeutic includes Factor VIII. In an embodiment, the protein therapeutic includes a Factor VIII bypass complex. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the protein therapeutic includes Factor IX. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure, a method of assigning viral-based gene therapy to a patient includes determining whether the patient has a genotype sensitizing the patient to persistent infection by a viral-based gene therapy vector by one or both of: (i) evaluating whether the patient has a mutation in the SMRT/NCOR2 gene associated with reduced SMRT/NCOR2 protein function, and (ii) evaluating whether the patient has a mutation in the interleukin-6 receptor (IL-6R) gene associated with reduced IL-6R function. A viral-based gene therapy is assigned to the patient if the patient has either a mutation in the SMRT/NCOR2 gene associated with reduced SMRT/NCOR2 protein function or a mutation in the IL-6R gene associated with reduced IL-6R function.

In an embodiment, determining whether the patient has a genotype sensitizing the patient to persistent infection by a viral-based gene therapy vector includes determining whether the patient has a mutation in the SMRT/NCOR2 or IL-6R genes associated with increased sensitivity to persistent infection by a viral-based gene therapy vector by, for example, obtaining a biological sample from the patient, and performing a genotypying assay to on the biological sample to determine whether the patient has a mutation in the SMRT/NCOR2 or IL-6R genes.

In some embodiments, the genotype sensitizing the patient to persistent infection by a viral-based gene therapy vector includes mutations in both copies of the patient's SMRT/NCOR2 gene that reduce the protein function of the encoded SMRT/NCOR2 proteins by at least 75% relative to the wild type SMRT/NCOR2 protein function.

In some embodiments, the genotype sensitizing the patient to persistent infection by a viral-based gene therapy vector includes a mutation in at least one copy of the patient's IL-6R gene that causes IL-6R haplodeficiency. In an embodiment, the mutation in the at least one copy of the patient's IL-6R gene is a missense mutation in the IL-6R gene.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure, a method of assigning viral-based gene therapy to a patient includes determining whether the patient has a genotype sensitizing the patient to persistent infection by a viral-based gene therapy vector by evaluating whether the patient has a mutation in the SMRT/NCOR2 gene associated with reduced SMRT/NCOR2 protein function. A viral-based gene therapy is assigned to the patient if the patient has a mutation in the SMRT/NCOR2 gene associated with reduced SMRT/NCOR2 protein function.

In some embodiments, determining whether the patient has a genotype sensitizing the patient to persistent infection by a viral-based gene therapy vector includes determining whether the patient has a mutation in the SMRT/NCOR2 gene associated with increased sensitivity to persistent infection by a viral-based gene therapy vector by, for example, obtaining a biological sample from the patient, and performing a genotypying assay to on the biological sample to determine whether the patient has a mutation in the SMRT/NCOR2 gene.

In some embodiments, the genotype sensitizing the patient to persistent infection by a viral-based gene therapy vector includes mutations in both copies of the patient's SMRT/NCOR2 gene that reduce the protein function of the encoded SMRT/NCOR2 proteins by at least 75% relative to the wild type SMRT/NCOR2 protein function.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure, a method of assigning viral-based gene therapy to a patient includes determining whether the patient has a genotype sensitizing the patient to persistent infection by a viral-based gene therapy vector by evaluating whether the patient has a mutation in the interleukin-6 receptor (IL-6R) gene associated with reduced IL-6R function. A viral-based gene therapy is assigned to the patient if the patient has a mutation in the IL-6R gene associated with reduced IL-6R function.

In some embodiments, determining whether the patient has a genotype sensitizing the patient to persistent infection by a viral-based gene therapy vector includes determining whether the patient has a mutation in the IL-6R gene associated with increased sensitivity to persistent infection by a viral-based gene therapy vector by, for example, obtaining a biological sample from the patient, and performing a genotypying assay to on the biological sample to determine whether the patient has a mutation in the IL-6R gene.

In some embodiments, the genotype sensitizing the patient to persistent infection by a viral-based gene therapy vector includes a mutation in at least one copy of the patient's IL-6R gene that causes IL-6R haplodeficiency. In an embodiment, the mutation in the at least one copy of the patient's IL-6R gene is a missense mutation in the IL-6R gene.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure a method for assigning a treatment for a disease associated with insufficient level of an enzymatic activity in a patient includes determining whether the patient has a genotype sensitizing the patient to persistent infection by a viral-based gene therapy vector by one or both of: (i) evaluating whether the patient has a mutation in the SMRT/NCOR2 gene associated with reduced SMRT/NCOR2 protein function, and (ii) evaluating whether the patient has a mutation in the interleukin-6 receptor (IL-6R) gene associated with reduced IL-6R function. A viral-based gene therapy is assigned to the patient if the patient has either a mutation in the SMRT/NCOR2 gene associated with reduced SMRT/NCOR2 protein function or a mutation in the IL-6R gene associated with reduced IL-6R function. A treatment by administering a polypeptide having the enzymatic activity is assigned to the patient if the patient does not have either or both a mutation in the SMRT/NCOR2 gene associated with reduced SMRT/NCOR2 protein function or a mutation in the IL-6R gene associated with reduced IL-6R function.

In an embodiment, determining whether the patient has a genotype sensitizing the patient to persistent infection by a viral-based gene therapy vector includes determining whether the patient has a mutation in the SMRT/NCOR2 or IL-6R genes associated with increased sensitivity to persistent infection by a viral-based gene therapy vector by, for example, obtaining a biological sample from the patient, and performing a genotypying assay to on the biological sample to determine whether the patient has a mutation in the SMRT/NCOR2 or IL-6R genes.

In some embodiments, the genotype sensitizing the patient to persistent infection by a viral-based gene therapy vector includes mutations in both copies of the patient's SMRT/NCOR2 gene that reduce the protein function of the encoded SMRT/NCOR2 proteins by at least 75% relative to the wild type SMRT/NCOR2 protein function.

In some embodiments, the genotype sensitizing the patient to persistent infection by a viral-based gene therapy vector includes a mutation in at least one copy of the patient's IL-6R gene that causes IL-6R haplodeficiency. In an embodiment, the mutation in the at least one copy of the patient's IL-6R gene is a missense mutation in the IL-6R gene.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the protein therapeutic includes Factor VIII. In an embodiment, the protein therapeutic includes a Factor VIII bypass complex. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the protein therapeutic includes Factor IX. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure a method for assigning a treatment for a disease associated with insufficient level of an enzymatic activity in a patient includes determining whether the patient has a genotype sensitizing the patient to persistent infection by a viral-based gene therapy vector by evaluating whether the patient has a mutation in the interleukin-6 receptor (IL-6R) gene associated with reduced IL-6R function. A viral-based gene therapy is assigned to the patient if the patient has either a mutation in the IL-6R gene associated with reduced IL-6R function. A treatment by administering a polypeptide having the enzymatic activity is assigned to the patient if the patient does not have a mutation in the IL-6R gene associated with reduced IL-6R function.

In an embodiment, determining whether the patient has a genotype sensitizing the patient to persistent infection by a viral-based gene therapy vector includes determining whether the patient has a mutation in the IL-6R gene associated with increased sensitivity to persistent infection by a viral-based gene therapy vector by, for example, obtaining a biological sample from the patient, and performing a genotypying assay to on the biological sample to determine whether the patient has a mutation in the IL-6R gene.

In some embodiments, the genotype sensitizing the patient to persistent infection by a viral-based gene therapy vector includes a mutation in at least one copy of the patient's IL-6R gene that causes IL-6R haplodeficiency. In an embodiment, the mutation in the at least one copy of the patient's IL-6R gene is a missense mutation in the IL-6R gene.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the protein therapeutic includes Factor VIII. In an embodiment, the protein therapeutic includes a Factor VIII bypass complex. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the protein therapeutic includes Factor IX. In an embodiment, the encoded Factor IX polypeptide has an R338L amino acid change relative to the wild type Factor IX sequence. In an embodiment, the viral-based gene therapy vector includes a Factor IX polynucleotide encoding the Factor IX polypeptide and the Factor IX polynucleotide includes a nucleic acid sequence CS06.

In another aspect of the present disclosure a method for assigning a treatment for a disease associated with insufficient level of an enzymatic activity in a patient includes determining whether the patient has a genotype sensitizing the patient to persistent infection by a viral-based gene therapy vector by evaluating whether the patient has a mutation in the SMRT/NCOR2 gene associated with reduced SMRT/NCOR2 protein function. A viral-based gene therapy is assigned to the patient if the patient has either a mutation in the SMRT/NCOR2 gene associated with reduced SMRT/NCOR2 protein function. A treatment by administering a polypeptide having the enzymatic activity is assigned to the patient if the patient does not have a mutation in the SMRT/NCOR2 gene associated with reduced SMRT/NCOR2 protein function.

In an embodiment, determining whether the patient has a genotype sensitizing the patient to persistent infection by a viral-based gene therapy vector includes determining whether the patient has a mutation in the SMRT/NCOR2 gene associated with increased sensitivity to persistent infection by a viral-based gene therapy vector by, for example, obtaining a biological sample from the patient, and performing a genotypying assay to on the biological sample to determine whether the patient has a mutation in the SMRT/NCOR2 gene.

In some embodiments, the genotype sensitizing the patient to persistent infection by a viral-based gene therapy vector includes mutations in both copies of the patient's SMRT/NCOR2 gene that reduce the protein function of the encoded SMRT/NCOR2 proteins by at least 75% relative to the wild type SMRT/NCOR2 protein function.

In some embodiments, the viral-based gene therapy vector is an adeno-associated virus (AAV) vector. In an embodiment, the AAV vector is a serotype 8 AAV (AAV8) vector. In some embodiments, the viral-based gene therapy vector includes a polynucleotide having a nucleic acid sequence encoding a therapeutic protein wherein the nucleic acid sequence encoding the therapeutic protein includes at least 10 CG dinucleotides. In some embodiments, the nucleic acid sequence encoding the therapeutic protein includes at least 25 CG dinucleotides, at least 30 CG dinucleotides, at least 35 CG dinucleotides, at least 40 CG dinucleotides, at least 50 CG dinucleotides, or any other number of CG dinucleotides between any two of these numbers.

In some embodiments, the patient has hemophilia A and the viral-based gene therapy vector encodes a Factor VIII polypeptide. In an embodiment, the protein therapeutic includes Factor VIII. In an embodiment, the protein therapeutic includes a Factor VIII bypass complex. In an embodiment, the encoded Factor VIII polypeptide is a B-domain deleted Factor VIII polypeptide. In an embodiment, the viral-based gene therapy vector includes a Factor VIII polynucleotide encoding the Factor VIII polypeptide, and the Factor VIII polynucleotide includes a nucleic acid sequence CS04. In an embodiment, the viral-based gene therapy vector includes a Factor VIII polynucleotide encoding the Factor VIII polypeptide, and the Factor VIII polynucleotide includes a nucleic acid sequence CS04+NG5+X5.

In some embodiments, the patient has hemophilia B and the viral-based gene therapy vector encodes a Factor IX polypeptide. In an embodiment, the protein therapeutic includes Factor IX. In an embodiment, the encoded Factor IX polypeptide has an R338L amino acid change relative to the wild type Factor LX sequence. In an embodiment, the viral-based gene therapy vector includes a Factor IX polynucleotide encoding the Factor IX polypeptide and the Factor IX polynucleotide includes a nucleic acid sequence CS06.

### EXAMPLES

### Example 1—Whole Exome Sequencing of Patients Treated with Adeno-Associated Virus Serotype 8-Factor IX (AAV8-FIX) Gene Therapy Reveals Potential Determinants of Persistent Transgene Expression

Safety and kinetics of FIX gene therapy in patients with hemophilia B was studied in a phase 1/II clinical trial to assess the impact of treatment on FIX activity levels and bleeding rates, and monitor the patients.

The first-in-human, phase/II, prospective, multicenter, open-label study (NCT01687608) employed a non-randomized (unblended single arm study), single ascending dose design to evaluate the safety and kinetics of FIX gene therapy construct FIX Padua gene therapy in adults with hemophilia B. The study was conducted in accordance with the standards of Good Clinical Practice and the principles of The Declaration of Helsinki. Ethical approval was obtained from the Institutional Review Boards of all clinical sites. The study protocol was reviewed by the US National Institutes of Health Recombinant DNA Advisory Committee, US Food and Drug Administration, and US National Heart, Lung and Blood Institute. Written informed consent for study participation and for whole exome sequencing was provided by all patients.

The study included male patients (aged 18 to 75 years) with hemophilia B (FIX≤2%; ≤1% for the first cohort), >150 exposure days to exogenous FIX concentrates, and either ≥3 hemorrhages per year requiring FIX replacement or regular use of FIX prophylaxis to prevent bleeding. Patients were excluded if they had evidence of liver disease, active viral infections with hepatitis, or poorly controlled HIV, or neutralizing antibodies against AAV8 (antibody titer <1:5) (see Table 3 for the complete list of eligibility criteria). Eligible patients were enrolled from January 2013 to July 2016 and underwent screening (see Supplemental Materials for a full list of laboratory screening assessments [Table S2], including methodology details for serum cytokine. AAV8 and AAV2 NAbs, and antigen-specific IFN-γ enzyme-linked immunospot [ELISPOT], assays).

TABLE 3
Study inclusion and exclusion criteria

| Inclusion criteria | | Exclusion criteria | |
|---|---|---|---|
| 1. | Males age18-75 years, inclusive | 1. | Bleeding disorder(s) other than |
| | | | hemophilia B |
| 2. | Established hemophilia B with ≥3 | 2. | Family history of inhibitor to FIX |
| | hemorrhages per year requiring treatment | | protein or personal laboratory evidence |
| | with exogenous FIX OR use of FIX | | of having developed inhibitors to FIX |
| | prophylaxis because of history of frequent | | protein at any time (≥0.6 Bethesda Units |
| | bleeding episodes | | on any single test) |
| 3. | Plasma FIX activity ≤2% (<1% for first | 3. | Documented prior allergic reaction to |
| | cohort; then per protocol) | | any FIX product |
| 4. | History of >150 exposure days to | 4. | AAV8 neutralizing antibodies titer |
| | exogenously administered FIX | | higher than 1:X |
| 5. | Normal prothrombin time (PT) | 5. | ELISPOT positive |
| 6. | Negative for active Hepatitis C virus | 6. | Serum markers indicating possible |
| | (HCV), defined as Hepatitis C virus | | autoimmune-mediated hepatitis: |
| | antibody negative and negative | | Antinuclear antibody (ANA) titer >1:180 |
| | (undetectable) polymerase chain reaction | | Anti-smooth muscle antibody assay results |
| | (PCR) test for plasma Hepatitis C virus | | ≥40 (Inova QUANTA LiteTM Actin |
| | ribonucleic acid (RNA) OR if Hepatitis | | immunoglobulin [IgG] enzyme-linked |
| | C virus antibody positive must have ≥2 | | immunosorbent assay [ELISA]); values of |
| | consecutive negative (undetectable) PCR | | 31-39 will be flagged as possibly abnormal |
| | tests for plasma HCV RNA at least 3 | | and PI and Medical Monitor will evaluate |
| | months apart, and negative at screening | | subject for eligibility |
| | | | Elevated anti-liver-kidney microsomal |
| | | | antibody type 1 (LKM1) titers |
| | | | Total IgG >2 × ULN (unless co-infected |
| | | | with human immunodeficiency virus [HIV]) |
| 7. | Men capable of fathering a child must | 7. | Received systemic immunosuppressive |
| | agree to use barrier contraception | | therapy within 72 hours prior to Study |
| | (combination of a condom and | | Day 0 |
| | spermicide) or limit sexual intercourse to | | |
| | post-menopausal, surgically sterilized, or | | |
| | contraception-practicing partners, for a | | |
| | minimum of 6 months after | | |
| | administration of AskBio009, or until | | |
| | AskBio009 genomes are no longer | | |
| | detected in the semen, whichever is | | |
| | sooner | | |
| 8. | Signed informed consent | 8. | Positive for Hepatitis B surface antigen |
| 9. | Willing and able to maintain a diary of | 9. | HIV infection AND: |
| | bleeding episodes and a personal record | | CD4 cell count <350 cells/mm3 (if CD4 is |
| | of home/outpatient FIX protein use | | not collected must have documentation |
| | | | negative for HIV infection within 30 days of |
| | | | screening), or |
| | | | Change in antiretroviral therapy regimen |
| | | | within 6 months prior to Study Day 0, or |
| | | | ALT or AST >1.5 × ULN |
| 10. | Willing and able to comply with the | 10. | Known immune disorder other than HIV |
| | other requirements of the protocol, | | (including myeloma or lymphoma) |
| | including provision of semen samples | | |
| | | 11. | Markers of hepatic inflammation or |
| | | | overt or occult cirrhosis as evidenced by |
| | | | one or more of the following: |
| | | | Platelet count <175,000/μL |
| | | | Albumin ≤3.5 g/dL |
| | | | Total bilirubin >1.5 × ULN and direct |
| | | | bilirubin ≥0.5 mg/dL |
| | | | Alkaline phosphatase >2.0 × ULN |
| | | | ALT or AST >2.0 × ULN (except for |
| | | | subjects who are HIV infected, per |
| | | | exclusion 8) |
| | | | Liver biopsy in the past indicating moderate |
| | | | or severe fibrosis (Metavir staging of 2 or |
| | | | greater) |
| | | | History of ascites, varices, variceal |
| | | | hemorrhage or hepatic encephalopathy |
| | | 12. | Absolute neutrophil count (ANC) <1000 |
| | | | cells/mm3 |
| | | 13. | Serum creatinine >1.5 mg/dL |
| | | 14. | Urine protein >1+ |
| | | 15. | Creatine kinase (CK) >2.5 × ULN |
| | | 16. | Body mass index >35 |
| | | 17. | Received an adeno-associated virus |
| | | | (AAV) vector previously |
| | | 18. | Received any other gene transfer agent |
| | | | within the 12 months prior to Study Day |
| | | | 0 |
| | | 19. | Received an investigational intervention |
| | | | or participated in another clinical trial |
| | | | within 30 days prior to enrollment or |
| | | | within 5 half-lives of the investigational |
| | | | drug in blood, whichever is longer |
| | | 20. | Received systemic antiviral and/or |
| | | | interferon therapy within 30 days prior |
| | | | to Study Day 0 (with the exception of |
| | | | treatment for HIV) |
| | | 21. | Received chemotherapy or biological |
| | | | therapy for treatment of neoplastic |
| | | | disease or other disorders within the 12 |
| | | | months prior to Study Day 0 |
| | | 22. | Clinically significant infection within 30 |
| | | | days prior to Study Day 0, as determined |
| | | | by the Investigator |
| | | 23. | Major surgery or an orthopedic surgical |
| | | | procedure within 3 months prior to |
| | | | Study Day 0 or planned within 6 months |
| | | | after Study Day 0 |
| | | 24. | Acute or chronic disease that would |
| | | | adversely affect subject safety or which |
| | | | could preclude subject from prophylactic |
| | | | administration of corticosteroid |
| | | | treatment as required by the protocol, or |
| | | | compliance or interpretation of study |
| | | | results, as determined by the Investigator |
| | | 25. | Recent history of psychiatric illness or |
| | | | cognitive dysfunction (including drug or |
| | | | alcohol abuse) that is likely to impair |
| | | | subject's ability to comply with |
| | | | protocol-mandated procedures, as |
| | | | determined by the Investigator |

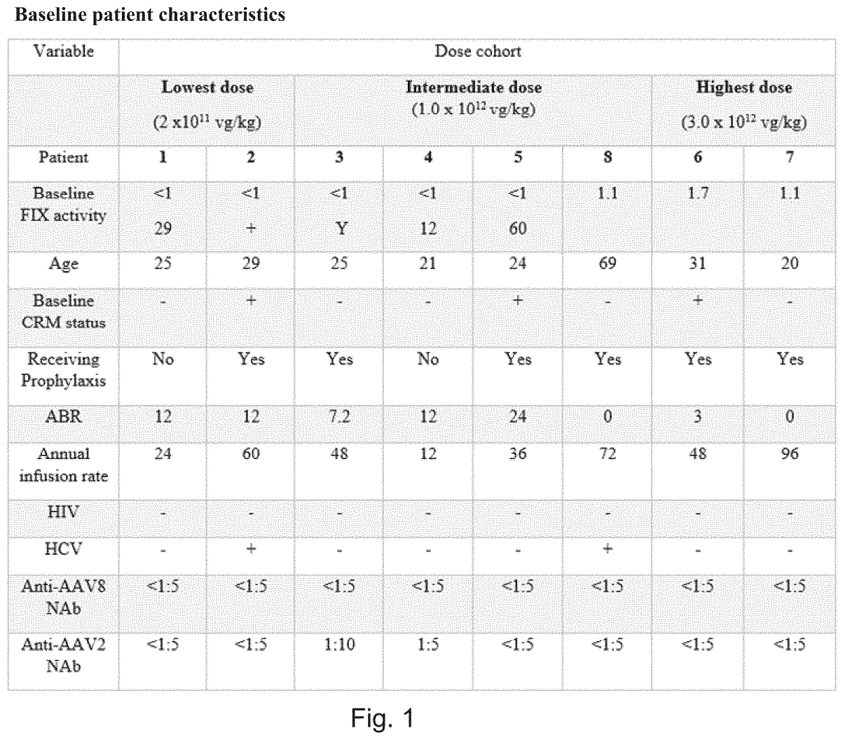

The FIX gene therapy expression cassette consisted of a self-complementary transgene flanked by AAV2-derived inverted terminal repeats (ITRs), liver specific transthyretin (TTR) promoter/% enhancers, and the hyperactive FIX (R338L) Padua variant (FIG. 1). Good Manufacturing Practice FIX gene therapy construct was manufactured at the University of North Carolina at Chapel Hill School of Medicine (UNC) Vector Core Facility using a triple plasmid transfection protocol of suspension HEK293 cells grown in the WAVE bioreactor system (GE Healthcare, Piscataway, N.J., USA). The vector titer was determined by Q-PCR and dot blot assay.

Enrolled patients were assigned to receive single i.v. infusions of FIX gene therapy construct in 1 of 3 ascending dose cohorts; 1) 2.0×10¹¹ vector genomes (vg)/kg; 2) 1.0×10¹² vg/kg 3) 3.0×10¹² vg/kg. Patients were permitted to receive standard of care hemophilia B treatment (including exogenous FIX protein for on-demand treatment of bleeding episodes and/or prophylaxis) as required during the study.

The primary outcome measures were assessment of adverse events by dose cohort and changes in laboratory evaluations from baseline. Secondary outcome measures included the monitoring of vector shedding in bodily fluids (blood, saliva, urine, stool and semen), as well as the assessment of systemic IRs (humoral and cellular) to the FIX Padua (R338L) transgene product and to AAV8 capsid proteins, at specified time points post infusion. Binding Ig antibodies against wt FIX and FIX Padua were also assayed. In addition, circulating tumor necrosis factor alpha (TNFα) and interleukin-6 (IL-6) levels in blood were measured pre- and 24-hours post FIX gene therapy construct infusion (further details in Supplemental Materials).

Secondary efficacy outcomes included changes in FIX activity and FIX protein levels from preinfusion baseline. FIX activity was measured in a standard one-stage FIX activity assay, using a Siemens BCS-XP automated analyzer and ellagic acid as the aPTT activator, performed at the central laboratory (Esoterix, Inc. Englewood, Colo.). This assay also formed the basis of the Bethesda Inhibitor assays, which were used to examine inhibition of clotting in wt FIX-containing plasma and in FIX Padua-containing plasma. Development of a transgene product-specific ELISA, to quantify FIX Padua protein from samples of patient plasma, has been described previously. Bleeding episode frequency and severity and use of exogenous FIX products during the study was also compared with data collected for the 12-month pre-study period.

Human Luminex kit was custom-designed by Millipore® (Merck Millipore, Darmstadt, Germany). The kit provided ready-to-use cocktails of the respective analytes as standard for the assay. The assay was performed accordingly to the manufacture's protocol. In brief, the standard was reconstituted with 250 μl deionized water to obtain a concentration of 10000 μg/ml for all cytokines. The vials were inverted multiple times for mixing and were kept on ice until use. Quality controls (low and high) were components of the kits and the respective quality control ranges were provided by the manufacturer. The two quality controls (low and high) were reconstituted with 250 μl deionized water (low and high). The samples were measured on a suspension array multiplex system (Bio-Plex® 200 System, BioRad®). The following parameters were analyzed: cytokines (interferon [IFN]α2, IFNγ, interleukin [IL]-10, IL-12p70, IL-13, IL-17A, IL-1α, IL-1B, IL-2, IL-4, IL-6, IL-8, monocyte chemoattractant protein-1 [MCP-1], macrophage inflammatory protein-1α [MIP-1α], tumor necrosis factorα [TNFα], transforming growth factor-β1 [TGF-0l], soluble CD40-ligand [sCD40L] and interferon gamma-induced protein 10 [IP-10]; liver parameters (gamma-glutamyl transferase [GGT], alkaline phosphatase, ferrum, ferritin, total protein, albumin, α-1-globulin, α-2-globulin, β-globulin, β-1-globulin, β-2-globulin, γ-globulin, α-1-fetoprotein and C-reactive protein [CRP]).

C57BL6 mice (Charles River Laboratories) were immunized with FIX gene therapy construct vector and with a next generation CpG-depleted vector using 4×10¹² vg/kg. Four weeks after challenge, anti-AAV8 neutralizing anti-bodies (NAbs) were assessed using the above described neutralizing antibody assay adapted to the mouse.

Root cause analyses, investigating the loss of transgene expression in some patients, included a comparison of AAV8 neutralizing antibody titers (Nab) in mice injected with CpG-rich versus CpG-depleted AAV8 vectors.

Assay for neutralizing Abs for AAV8 and AAV2 (in vitro transduction inhibition assays)

Serum was assayed for the presence of neutralizing antibodies (NAbs) against the AAV8 capsid during screening and regularly following FIX gene therapy construct infusion. At each time point, NAbs against AAV2 were also assayed; humans are the natural host of AAV2 and it was anticipated that approximately half of adult men screened would have pre-existing anti-AAV2 Nabs. An in vitro transduction inhibition assay was used as previously described to assay the potential for serum from a study subject to inhibit luciferase marker gene transfer in cell culture by AAV. Serial two-fold dilutions of subject serum were mixed 1:1 with AAV.luciferase and incubated for 2 hours at 37° C. and then used to infect Huh7 cells (which are permissive for infection by both AAV2 and AAV8) in tissue culture. Following 24 hours infection, luciferin was added to cells as a substrate for expressed luciferase, and luciferase activity quantified by luminometer. The highest dilution of the subject's serum that resulted in inhibition of ≥50% of luciferase activity (compared to control without serum) was recorded as the NAb titer.

Evaluation of T cell responses directed against AAV8 capsid and FIX Padua were evaluated at baseline and serially following FIX gene therapy construct infusion using IFNγ release Enzyme-linked Immunospot assay of peripheral blood mononuclear cells (peripheral blood T lymphocytes, PBMCs). A library of 15-mer peptides overlapping by 10 amino acids in sequence was generated (Mimotopes) to span the entire AAV8 capsid VP1 protein and was organized into 3 pools (AAV8 antigen pools 1-3). Also, a library of I5-mer peptides overlapping by 10 amino acids in sequence was generated (Mimotopes) to span the entire FIX R338L protein and was organized into 2 pools (FIX R338L antigen pools 1 and 2). As a positive control of the viability of the cells, leucoagglutinin PHA-L was tested at a final concentration of 1 μg/ml. As a positive control for polyclonal activation, PMA-ionomycin was tested at a final concentration of 5 ng/ml PMA and 2 mM ionomycin. For each plate a reference control was also run against CEF (Cellular Technology Limited) which comprises a pool of epitopes from CMV, EBV, and influenza viruses. Complete lymphocyte culture medium (RPMI with 10% FBS) was tested as the negative reactivity control. The day prior to anticipated cell culture, multiwell plates were coated with human IFNγ coating antibody (Mabtech) in sterile PBS overnight. On the day of cell culture, plates were washed with PBS and blocked with complete media. Fresh PBMCs from study subjects were adjusted to a concentration of 2×10 cells/ml in lymphocyte culture medium and 100 μl of cell suspension added to wells containing antigens (or controls) in an equal volume. After 18-24 hours stimulation (incubation) at 37° C., cell culture plates were washed and incubated with human IFNγ HRP detection antibody (Mabtech), followed by incubation with Aviden-HRP and subsequent incubation with AEC chromogenic reagent. Color development was stopped following five minutes incubation, plates dried, and human IFNγ activation counts quantified using AID Elispot Reader ELR03.

Quantitative real time polymerase chain reaction (qPCR) was used to detect FIX gene therapy construct vector genomes in whole blood, saliva, semen, urine, and stool on study day 1 post-treatment and at weekly intervals until 2 consecutive samples were below the limit of detection.

With written informed consent, genomic DNA from the 8 patients receiving FIX gene therapy construct was extracted for whole exome sequencing. The patient with sustained high level FIX expression during the study and for >4 years of follow-up (patient 5) was used as the index subject. A variant analysis was conducted to compare exome sequences from 7 patients who failed to sustain FIX transgene expression following FIX gene therapy construct infusion with the exome sequence from the index patient 5. Genomic DNA was extracted from EDTA-treated whole blood samples using the QIAamp DSP DNA Blood Mini Kit (Qiagen). Exome enrichment was performed using the TruSeq Rapid Capture Exome Library kit (Illumina) for each sample according to manufacturer's instructions. A fragmented DNA library was constructed for each sample and high-throughput sequencing was performed using Illumina HiSeq2000 sequencing platform in 100 bp paired-end mode. Mean sequencing depth of 125-180 reads per base satisfied the recommended sequencing depth for confident variant identification. The sequenced reads were aligned to GRCh37 using Burrows-Wheeler Aligner. Duplicate reads were marked with Picard Tools http://picard.sourceforge.net), while insertion/deletion realignment and base quality score recalibration was performed utilizing the GATK. Variant calling was performed using the SnpEff tool within GATK package. Variant prioritization was performed using the Ingenuity Variant Analysis tool (Qiagen). Variations between the whole exome sequence of patient 5 and the other subjects were evaluated using Combined Annotation Dependent Depletion (CADD) as a method to score likely deleteriousness of single nucleotide variants and insertions/deletions in the human genome. CADD provides a framework that integrates multiple variant annotation tools into a single metric by contrasting variants that survived natural selection with simulated mutations. Variants that are less likely to be observed (not having survived natural selection) receive a higher score, which correlates to how likely the variant is to be deleterious.

To evaluate the potential of CpG oligodeoxynucleotide sequences in the FIX gene therapy construct vector to serve as putative innate immunologic signals capable of augmenting adaptive anti-AAV immune responses, additional gene therapy vectors expressing codon optimized FIX cDNAs were generated. All of these were scAAV8 vectors packaging the FIX Padua coding sequences flanked by the same AAV2 ITR sequence, with expression driven by the same TTR promoter/enhancer and transcriptional regulatory elements. The vectors differed only in the number of CpG elements in the FIX Padua expression cassette, detailed as follows: 99 CpG in FIX gene therapy construct; zero CpG in vector ODN0 (Blue02); 3 CpG in vector ODN3. Additionally, the Gray01 gene sequence was synthesized using the codon optimized FIX gene sequence reported by Nathwani et al. (Self-complementary adeno-associated virus vectors containing a novel liver-specific human factor IX expression cassette enable highly efficient transduction of murine and nonhuman primate liver. Blood 2006107:2653-61.)

All animal experiments were approved by the Institution Animal Care and Use Committee of. All animal experiments were performed in hemostatically normal male C56B/6 mice. At 8-10 weeks of age, mice received a single tail vein injection of 4×10¹² vg/kg of either FIX gene therapy construct or of one of the CpG-reduced scAAV8 FIX expression vectors (n=6-8 mice/treatment group/experiment). Three separate clinical production lots of FIX gene therapy construct were examined. Mice were sacrificed at day 26 following vector infusion. Blood was collected to examine the formation of anti-AAV8 neutralizing antibodies (NAb) as a marker of whether adaptive immunity (antibody production) was boosted by the enrichment of TLR9 ligand CpG in the gene therapy vector.

Results

Eight males aged 20-69 years (mean [SD] age 30.5 [15.98] years; 6/8 aged <30 years) were eligible for study inclusion at screening and were enrolled into 1 of 3 dose cohorts (FIG. 1). Seven patients were Caucasian and 1 was Black/African American; 3 patients were positive for FIX antigen cross-reactive material (CRM). No patient had serologic evidence of active HIV or HCV (2 had anti-HCV antibodies, but all were negative for active HCV RNA by PCR). All had undetectable levels of AAV8 NAbs, but NAbs against AAV2 were detected in 2 patients (titers: 1:10 and 1:5, respectively) (FIG. 1). A circulating peripheral blood mononuclear cell (PBMC) response to AAV8 was detected in patient 7 at screening.

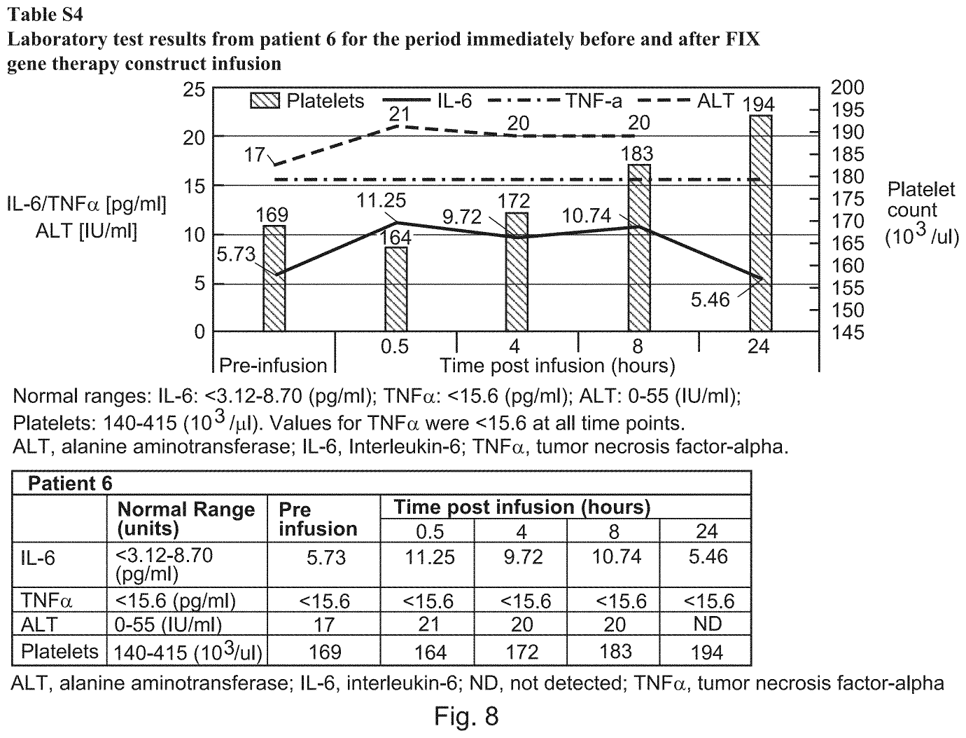

No abnormalities in vital signs or serum/hematologic parameters were reported during systemic FIX gene therapy construct infusion or in the 8-hour post-infusion period. One patient in the lowest dose cohort had a Grade 1 hypersensitivity reaction, which resolved within 30 minutes. No patient had his infusion interrupted. An asymptomatic elevation of serum IL-6 was reported in 1 patient in the highest dose cohort between 4 and 8 hours post-infusion, returning to baseline 24 hours post-infusion (FIG. 8).

Four SAEs were reported in 3 patients: rhabdomyolysis, bacterial infection of the tonsil, tonsillar hemorrhage, and squamous cell carcinoma of the tonsil (narrative information on this last event is provided in Supplemental Information); all were considered unrelated to FIX gene therapy construct. Ten AEs (in 4 patients) were considered as possibly related to the study treatment. Of these, 7 were mild in severity (fatigue, feeling flushed, headache, influenza-like symptoms, ankle swelling, elevated liver enzymes, and hypertension) and 3 were moderate (elevated liver enzymes, abscess, and fatigue).

Vector genomes were measured in urine, semen, saliva, stool, and whole blood, with the peak concentrations and duration of shedding broadly displaying a dose-dependence across the 3 cohorts (Table 4). With the exception of whole blood, no patient demonstrated shedding in body fluids after week 4 (semen, stool) to week 5 (saliva, urine). The vector signal from blood was lost in cohorts 1 and 2 but remained above the detection limit at the 12-month visit in the highest dose cohort.

TABLE 4
Shedding of wt FIX vector genomes in bodily fluids

| | Duration (days) | | |
|---|---|---|---|
| | Cohort 1 | Cohort 2 | Cohort 3 |
| | (2.0 × 10¹¹ | (1.0 × 10¹² | (3.0 × 10¹² |
| Sample | vg/kg) | vg/kg) | vg/kg) |
| Blood | 155.0 | 330.0 | NR |
| Saliva | 11.5 | 23.8 | 35.5 |
| Semen | 8.5 | 22.3 | 26.0 |
| Stool | 18.5 | 21.7 | 28.5 |

NR, not reached

Mean number of days that WT FIX genomes were detected in bodily fluids by dosing cohort. For whole blood samples taken from patients in the highest dosing cohort, WT FIX genomes remained above the level of detection at the 12-month visit.

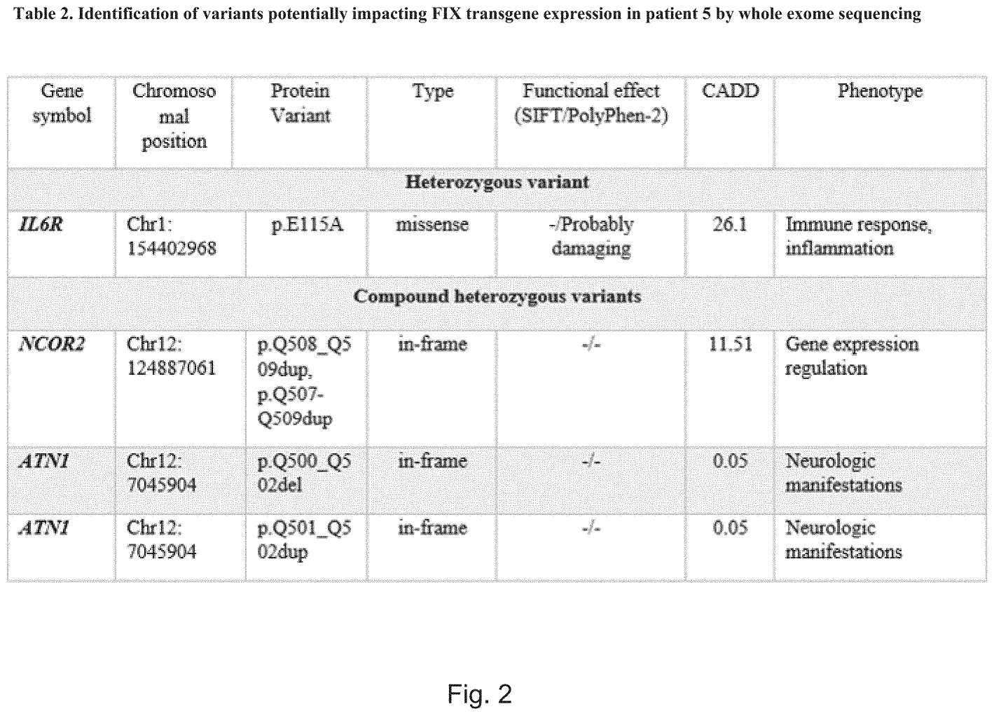

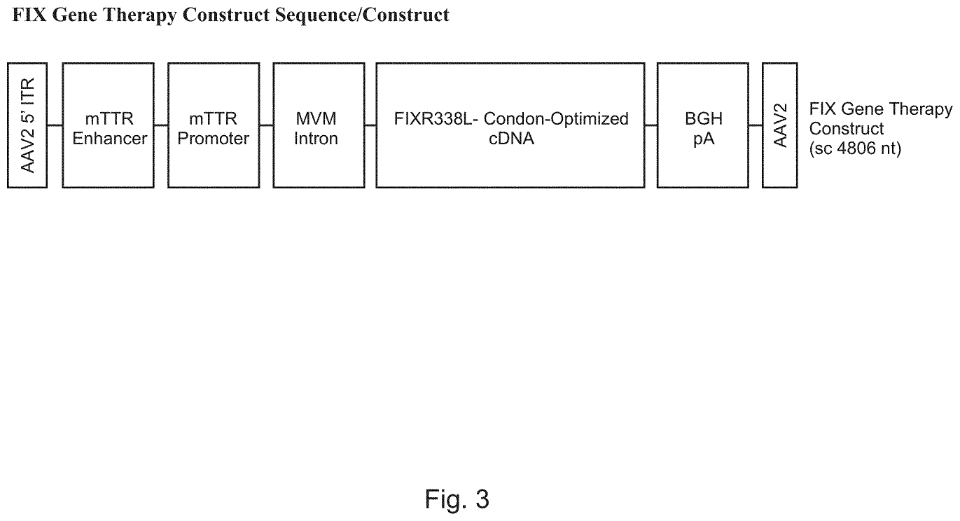

With the exception of the first patient treated in cohort 1, all patients demonstrated vector-derived hFIX expression, which was evident by weeks 1-2 after FIX gene therapy construct infusion in the higher dose cohorts (FIG. 2) and occurred without evidence of either thrombogenicity or cellular and humoral IRs to FIX Padua. FIX gene therapy construct-derived FIX expression was dose-dependent (FIGS. 2 and 3). The mean (range) peak FIX activity was 3.5% (3-4%) in cohort 1, 13% (3-25%) in cohort 2 and 45% (32-58%) in cohort 3. At the time of peak expression, circulating coagulation activity derived from gain-of-function FIX Padua (rather than wild type FIX) was demonstrated by the FIX-specific activity in patients who were FIX CRM negative at baseline (FIG. 2). Employing a FIX Padua-specific ELISA corroborated transgene-specific expression for all (CRM+ and CRIM−) patients, as the FIX Padua antigen levels correlated with the measured FIX activities but not with the FIX antigen levels measured with a standard FIX ELISA assay (FIG. 2).

In cohort 1, patient 2 achieved 2-3% FIX activity by week 4 and temporarily discontinued prophylaxis. All 4 patients in cohort 2 experienced significant levels of FIX activity, but with considerable interpatient variability. Patient 3 demonstrated FIX activity with a peak of 16.9% at week 11, but experienced an acute loss of FIX expression at week 12 and subsequently resumed prophylactic treatment. Patient 4 had an initial peak FIX activity level of ˜7%, which subsequently fell between weeks 6 and 7; he also resumed replacement therapy. Patient 8 received prophylactic corticosteroids; FIX activity of 2-3% was attained for the first 6 months. Patient 5 achieved FIX activity of ˜20% at week 7, and was the only patient with sustained activity for >4 years' follow-up, achieved in the absence of bleeding or FIX protein infusions (FIG. 2). Although both patient 3 and patient 4 lost >80% of peak expression acutely (over ˜2 weeks), neither patient demonstrated increased liver transaminases, T cell activation in response to vector or transgene (antigen-specific IFN-γ ELISPOT), development of inhibitors or non-neutralizing antibodies to FIX or to FIX Padua or other symptoms/laboratory perturbations coincident with this loss.

In the highest dose cohort, patient 6 achieved peak FIX activity of 58.5% by week 6, coinciding with an increase in AAV capsid-directed T cell activation. A steep fall in FIX activity occurred at week 7, accompanied by an increase in liver transaminases and further T cell activation. Per protocol, treatment with prednisone was initiated within 72 hours, resulting in rapid normalization of liver enzymes and ELISPOT results. FIX activity did not recover, and the patient resumed prophylactic FIX infusions. Patient 7 exceeded 30% FIX activity by week 5. He was started on prednisone after experiencing a slight elevation in liver enzymes together with a sudden loss of FIX activity at week 6: however, FIX activity continued to decline (FIG. 2).

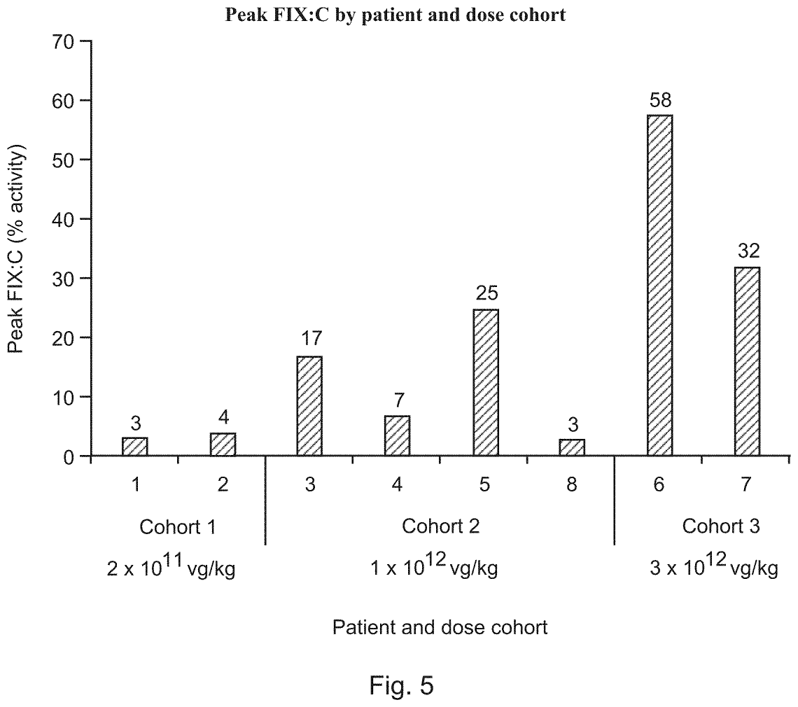

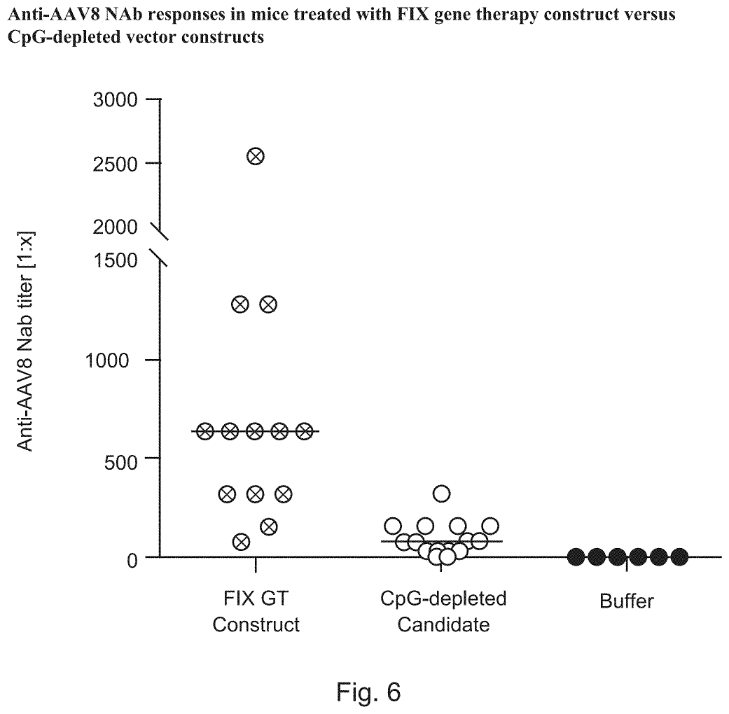

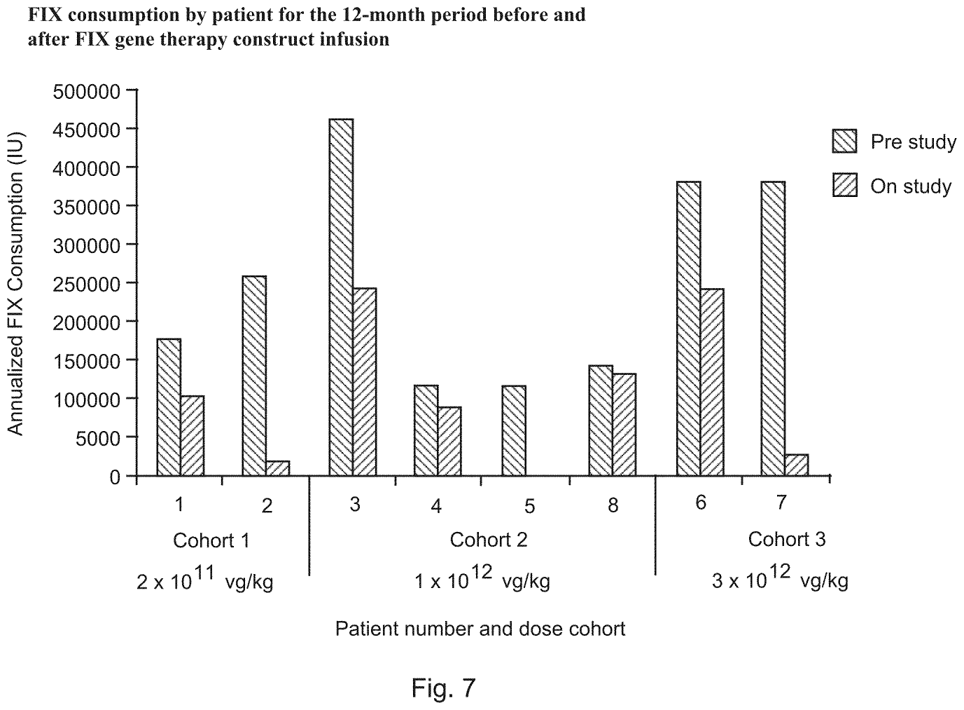

During the 12-month follow-up period after FIX gene therapy construct infusion, which included periods of peak factor expression, exogenous FIX consumption appears to be reduced compared with the previous year (annualized use of FIX was reduced in 6/8 patients and the number of FIX infusions per month in 7/8 patients). The largest reductions in factor consumption appeared to occur in patients from the highest dose cohort (FIG. 5).

AAV8-Specific T Cell Responses Against the Vector

AAV8-specific cytotoxic T cell responses are considered to kill transduced hepatocytes. However, only the two patients in the highest dose cohort had strong ELISPOT signals for AAV8 capsid-reactive T cells and these findings were not accompanied by AEs (FIG. 2). The initiation of corticosteroid therapy was associated with immediate normalization of the IFN-γ ELISPOT in patient 6, but this signal remained elevated for weeks after initiation of prednisone in patient 7.

Systemic corticosteroid administration initiated in response to ALT elevations in patients 6 and 7, and as prophylaxis in patient 8, did not serve to maintain FIX activity levels in these patients (FIG. 2).

Assaying patients' plasma samples for 18 cytokines and 15 liver parameters up to 5-8 months after dosing, did not reveal any conclusive hint as to why 1 only patient 5 selectively showed stable FIX Padua expression for more than 4 years (data not shown).

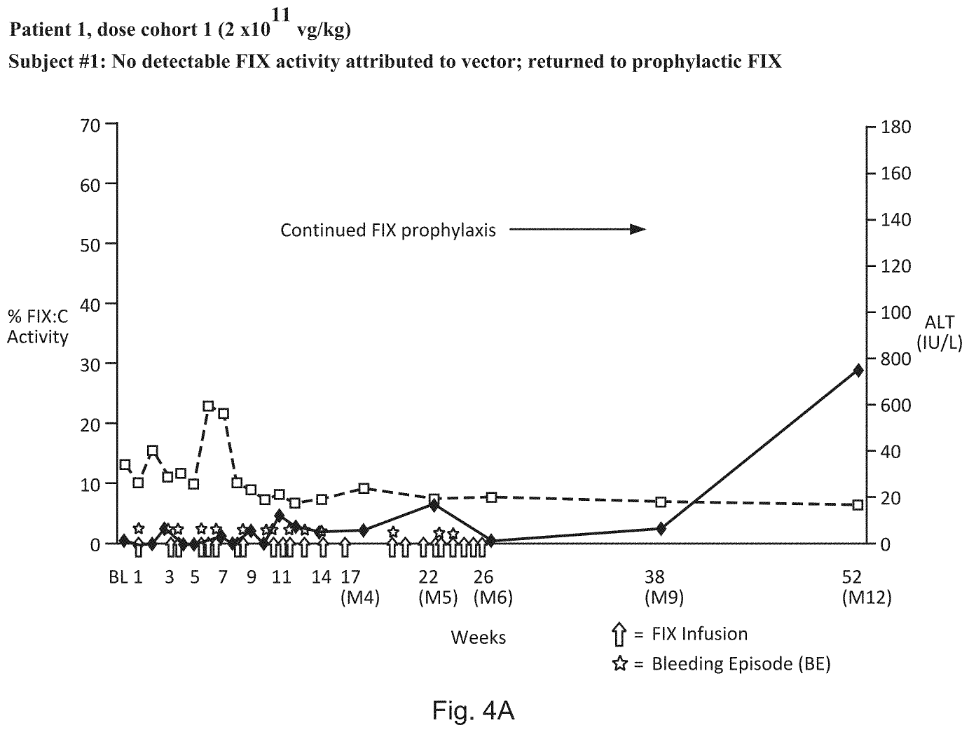

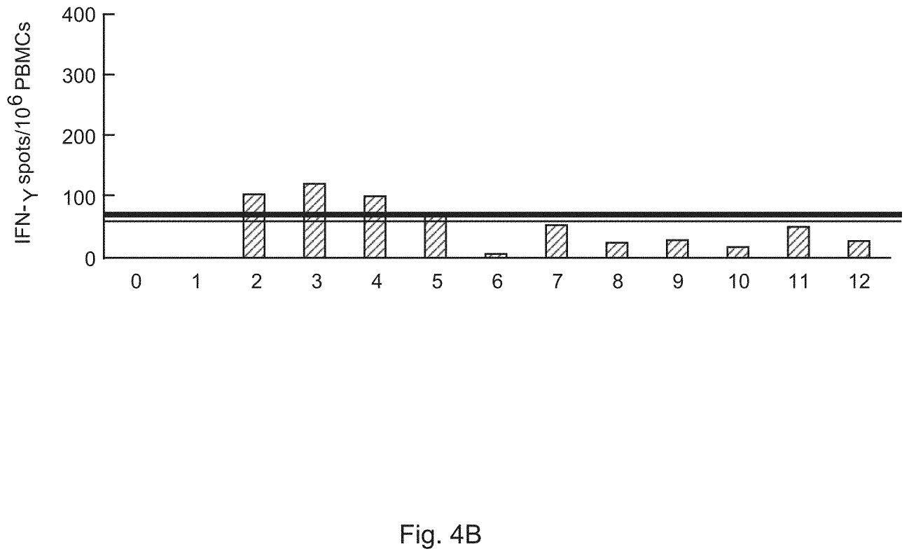

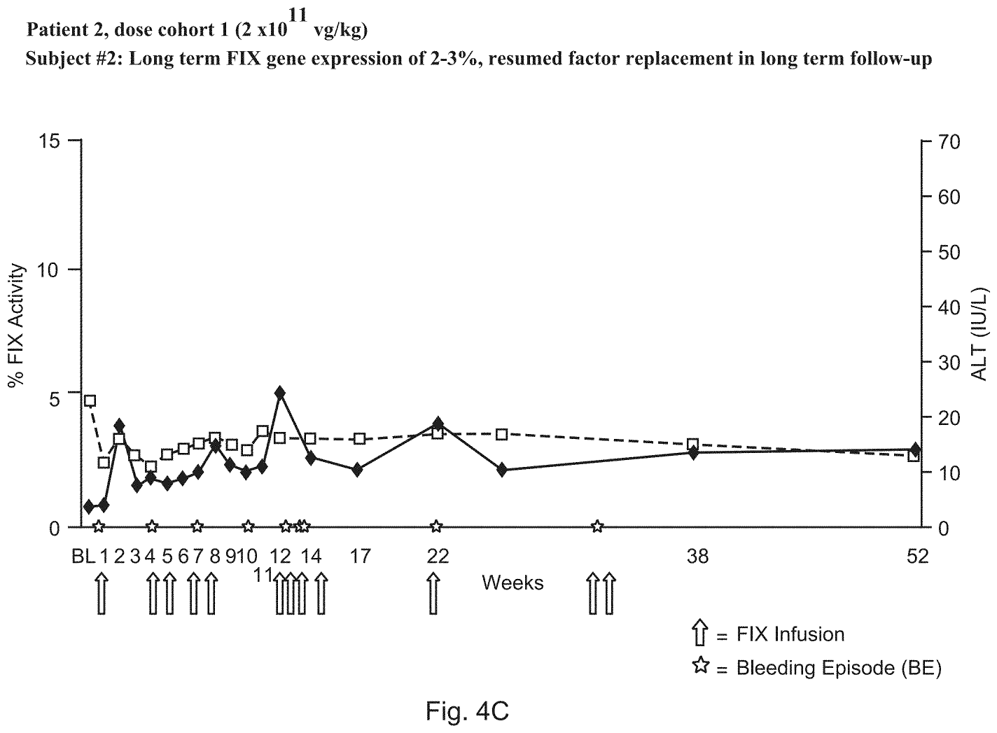

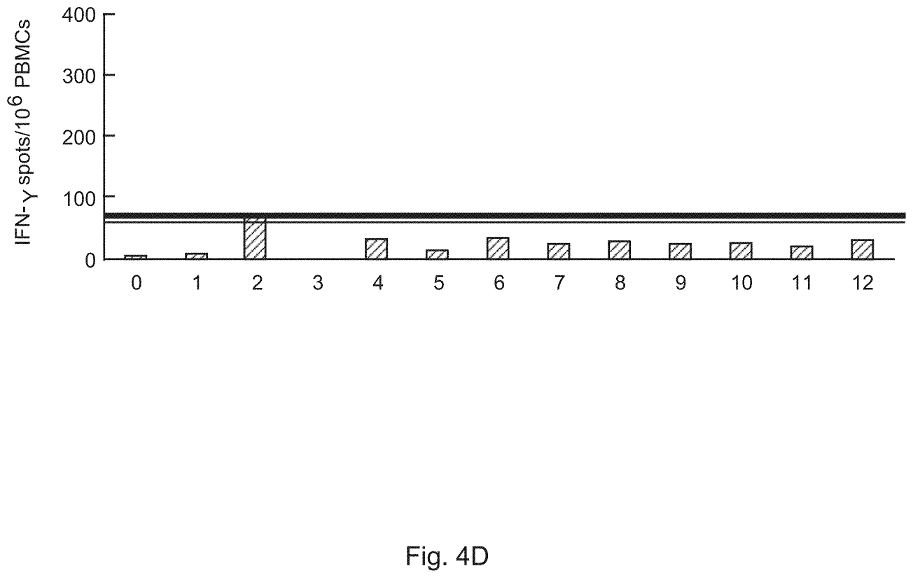

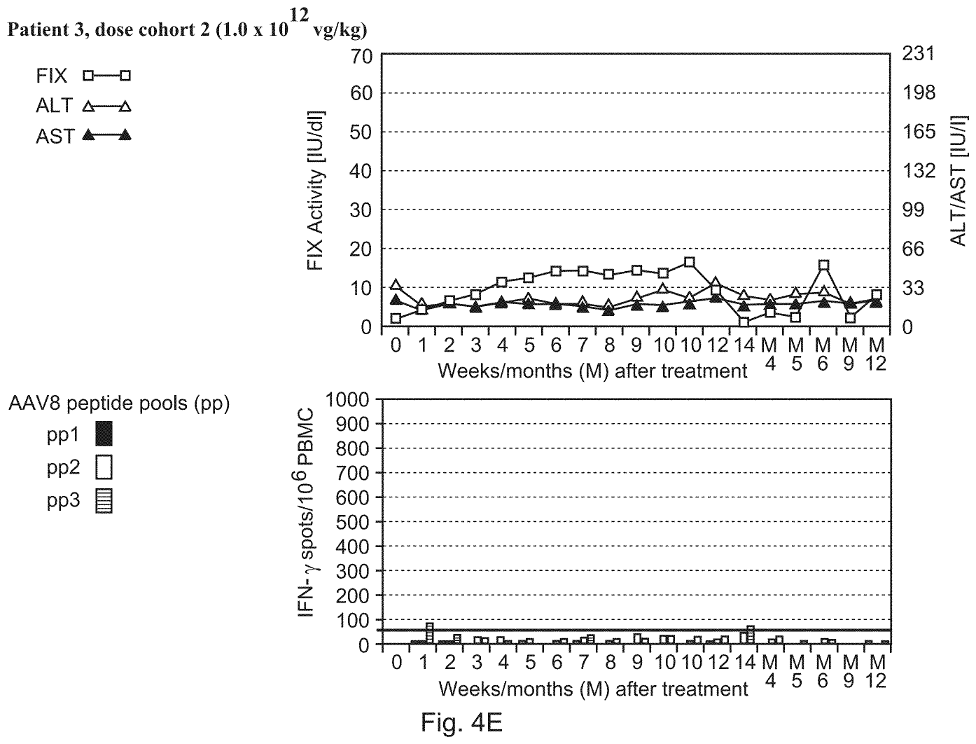

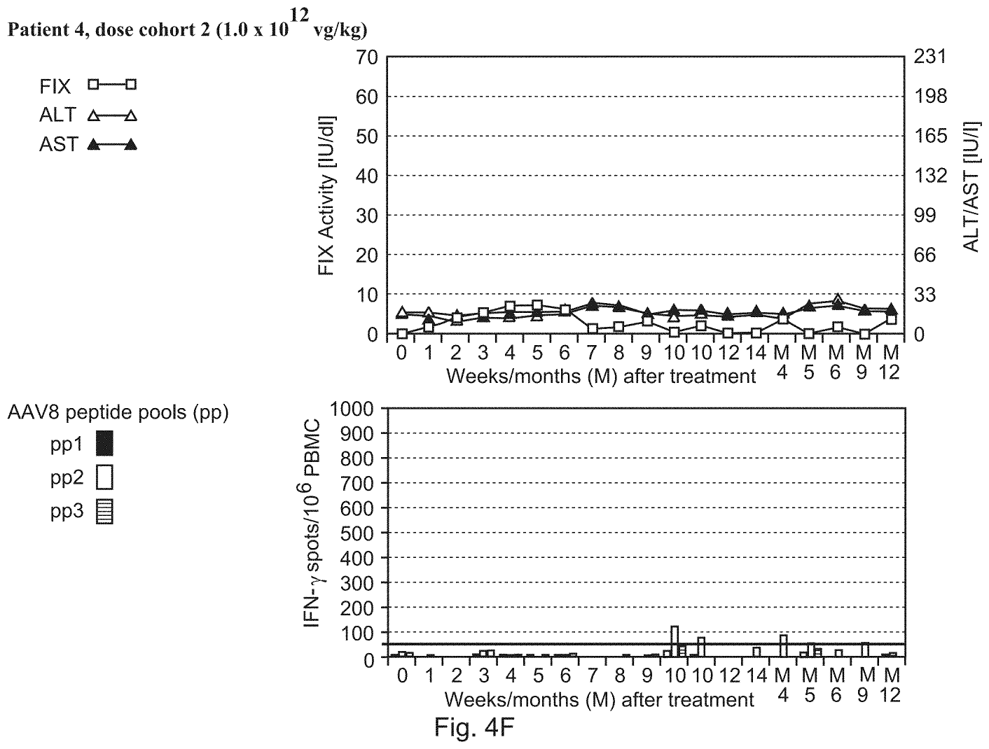

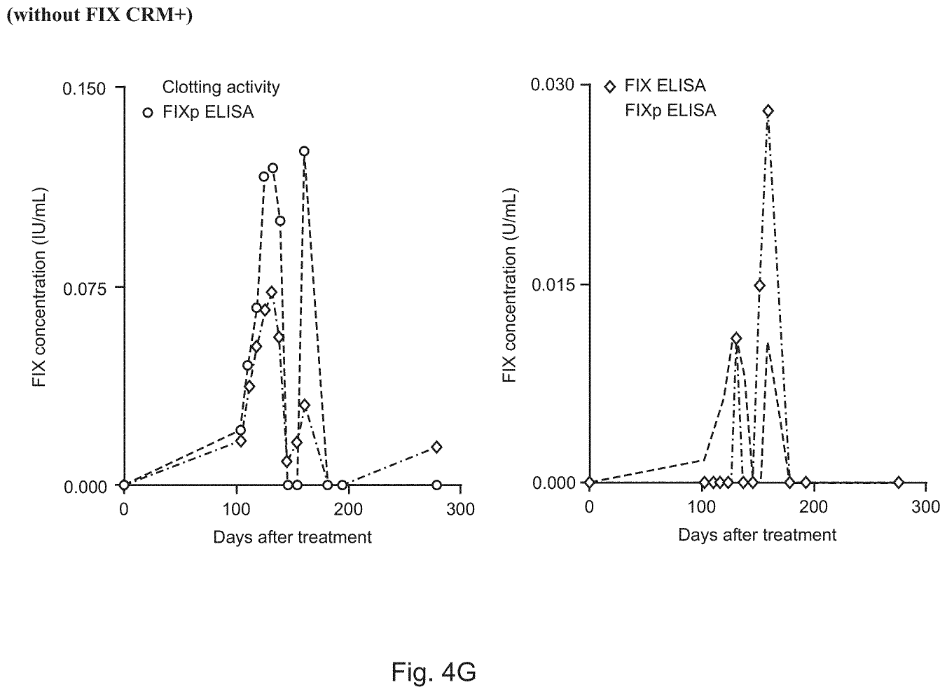

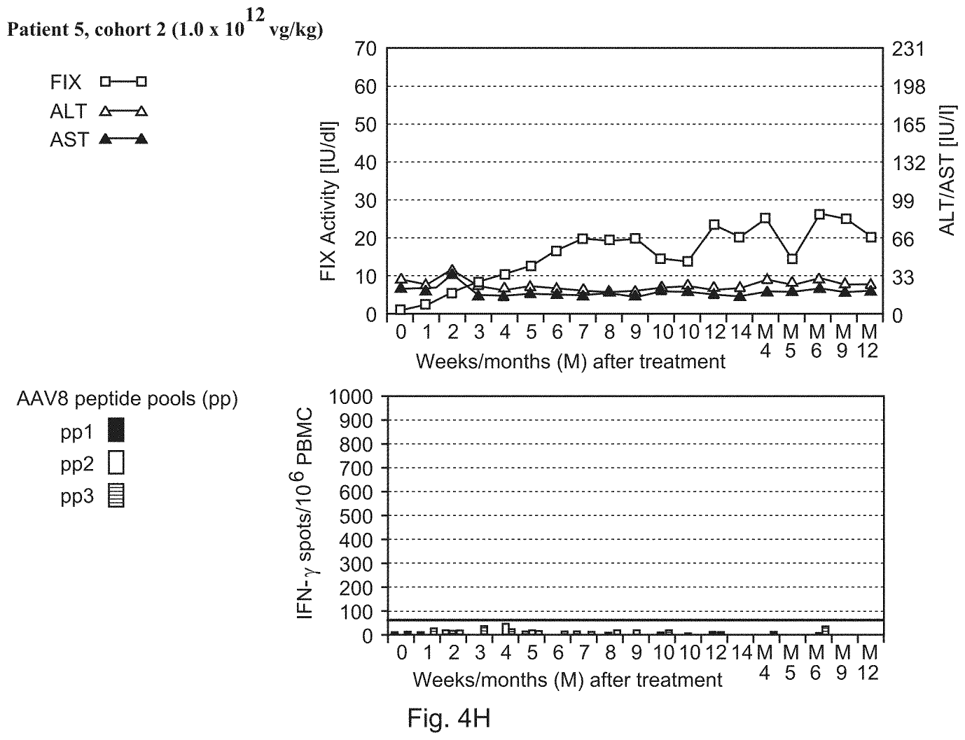

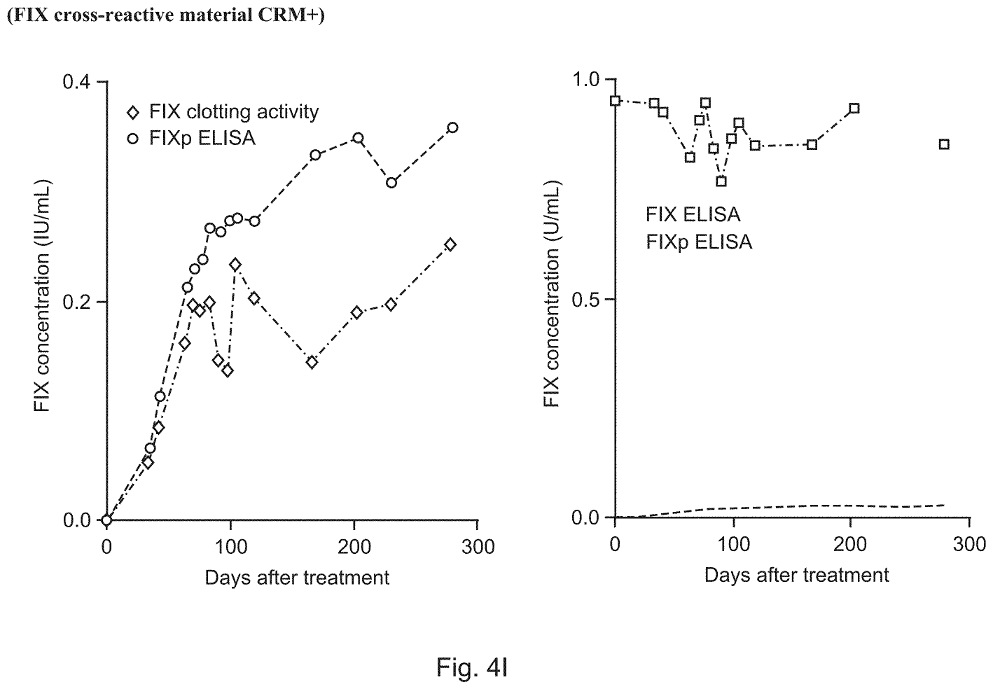

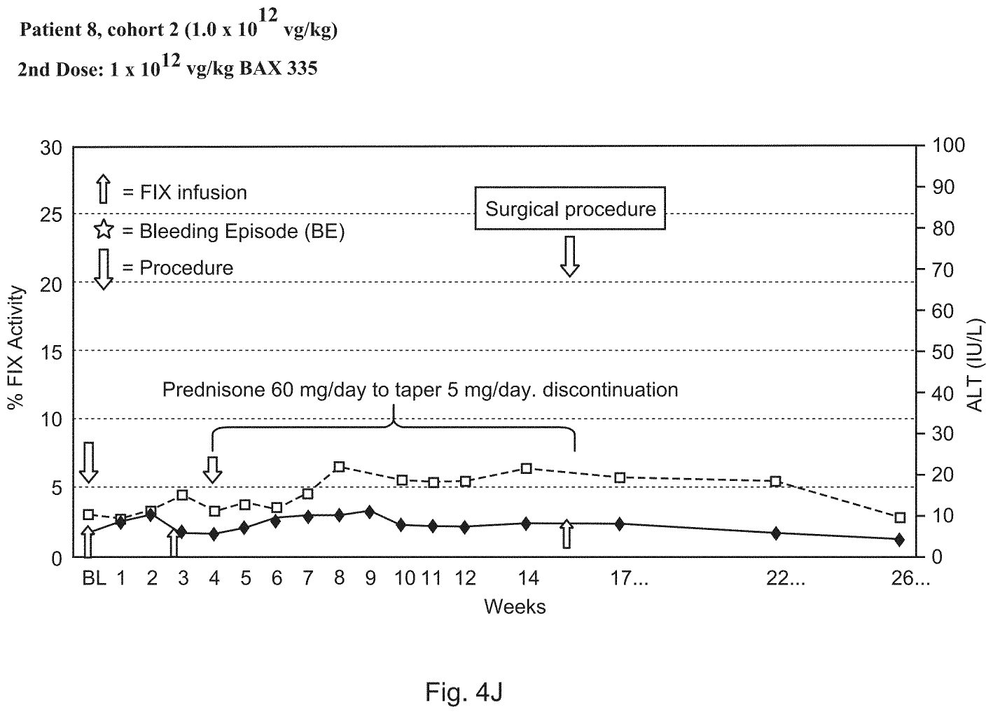

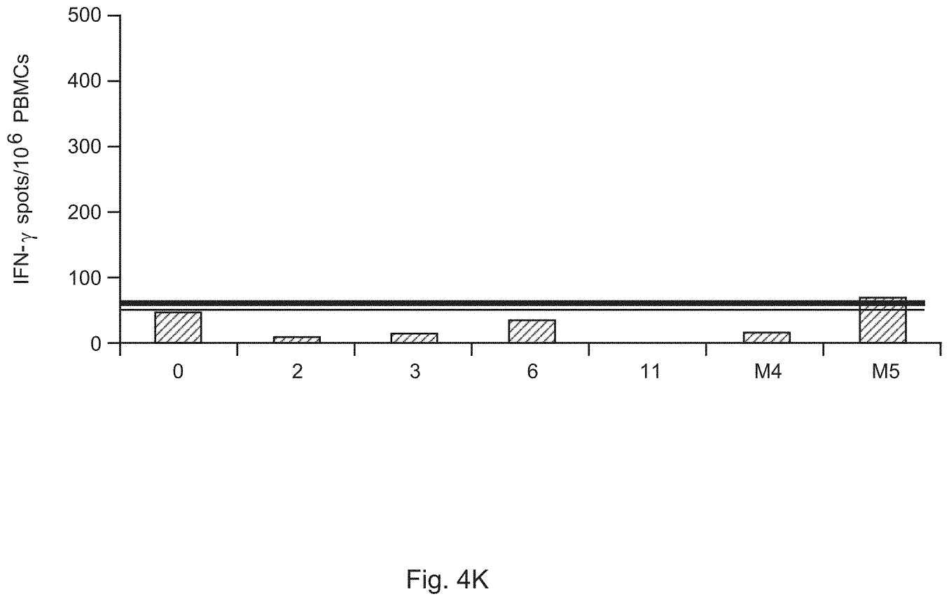

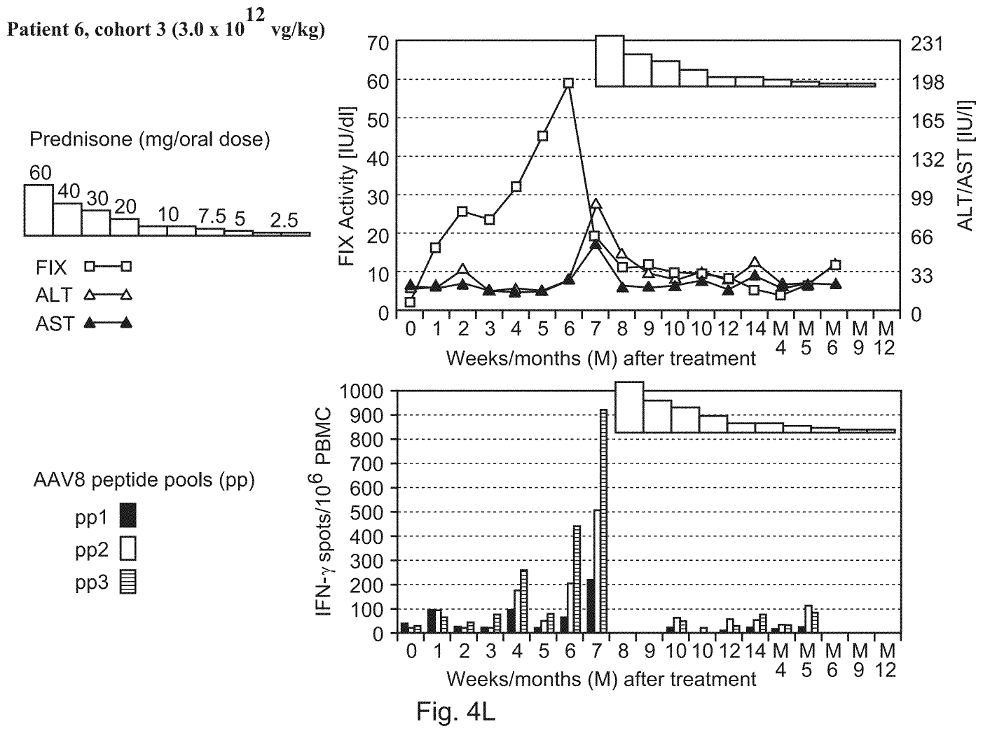

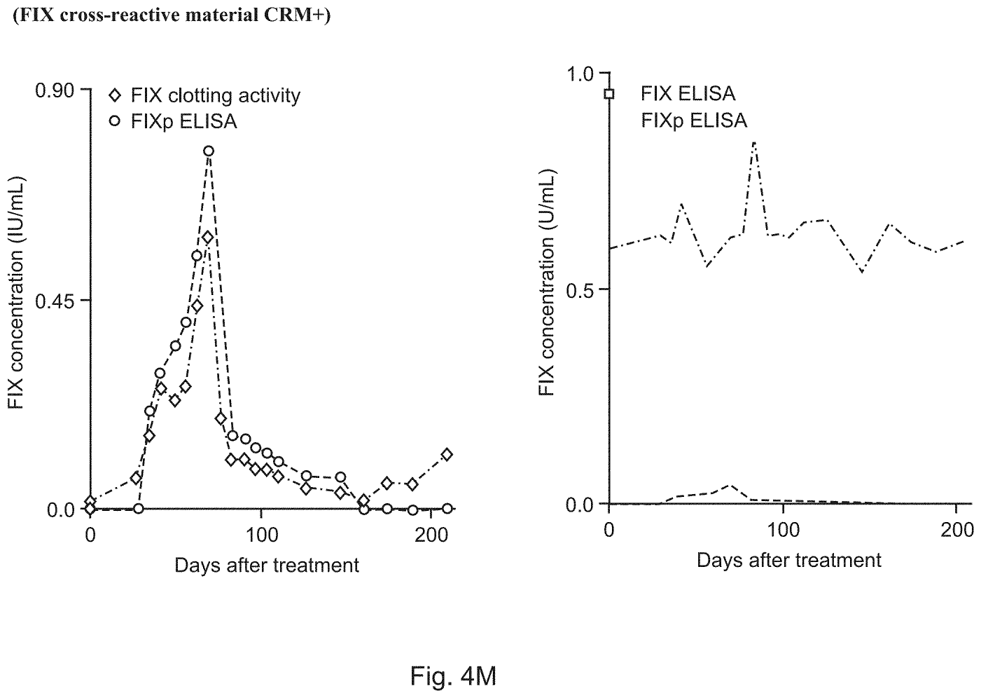

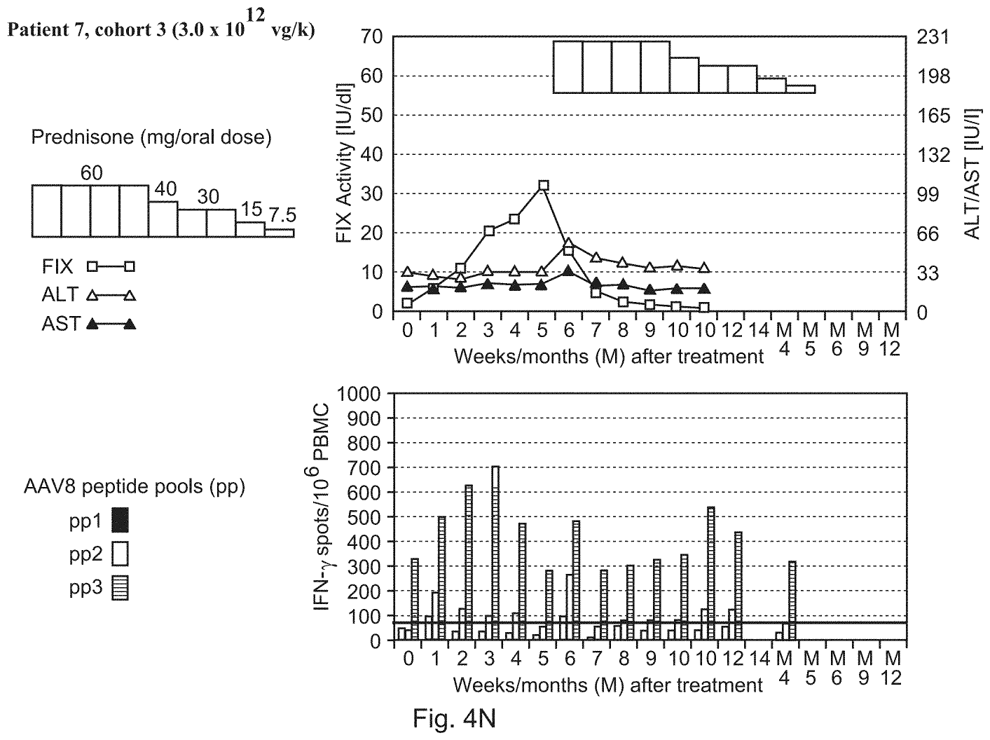

Vectorized expression cassettes harboring CpG clusters may activate the innate immune system via toll like receptor 9 (TLR9) with a potentially negative impact on transgene expression. Since the FIX Padua transgene in FIX gene therapy construct contains 99 CpG motifs giving rise to CpG islands of elevated CpG dinucleotide density. This hypothesis was tested in an animal model where a vector-dependent activation of the innate immune system was analyzed indirectly by measuring the titers of NAbs against AAV8. When animals were challenged with FIX gene therapy construct or a FIX gene therapy construct-like construct bearing a CpG-depleted FIX Padua transgene, the NAb titers against AAV8 were increased in animals treated with FIX gene therapy construct (FIG. 4). These data suggest that increased numbers of CpG motifs could indeed drive innate immune signaling capable of augmenting vector-targeted adaptive immunity.

To understand how the potential immunogenicity of the vector could translated into the stable expression of patient 5, it was hypothesized that a genetic variation could have contributed to the failure of patient 5 to mount an anti-AAV8 immune response facilitating the demonstrated long-term transgene expression. Therefore, potential genetic variations within the coding regions of the genome in all patients by whole exome sequencing were investigated.

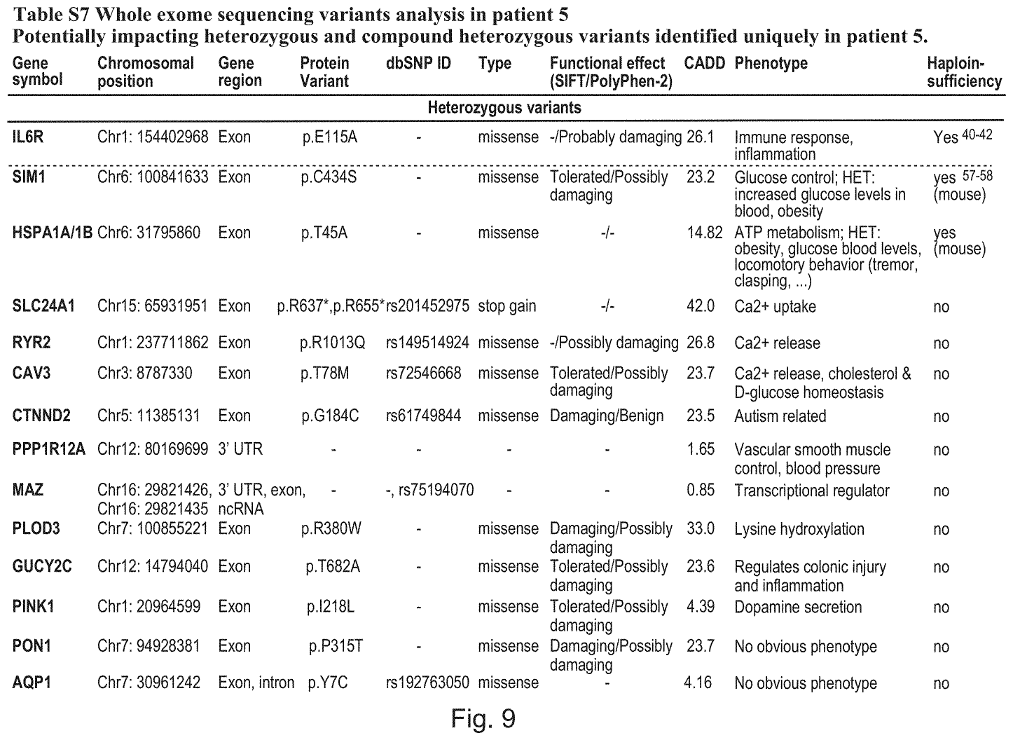

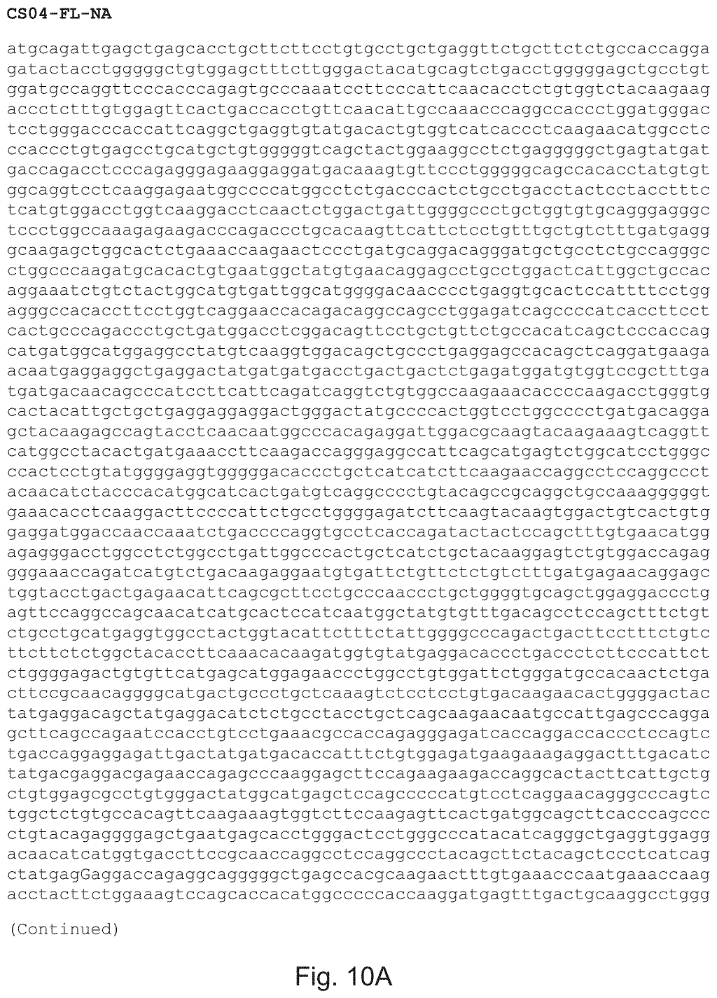

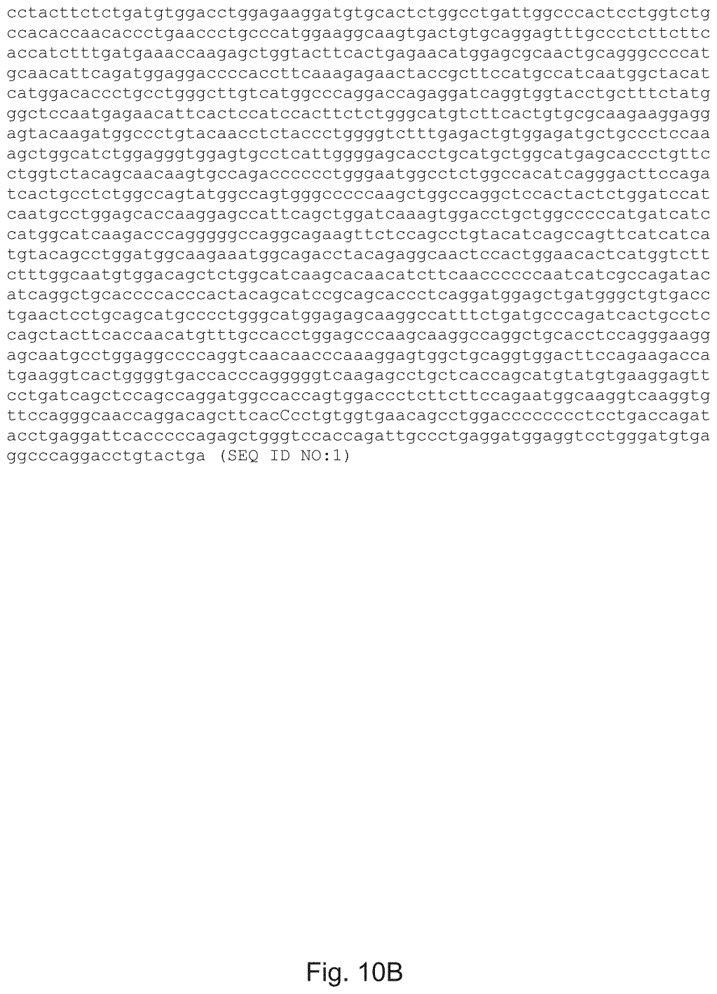

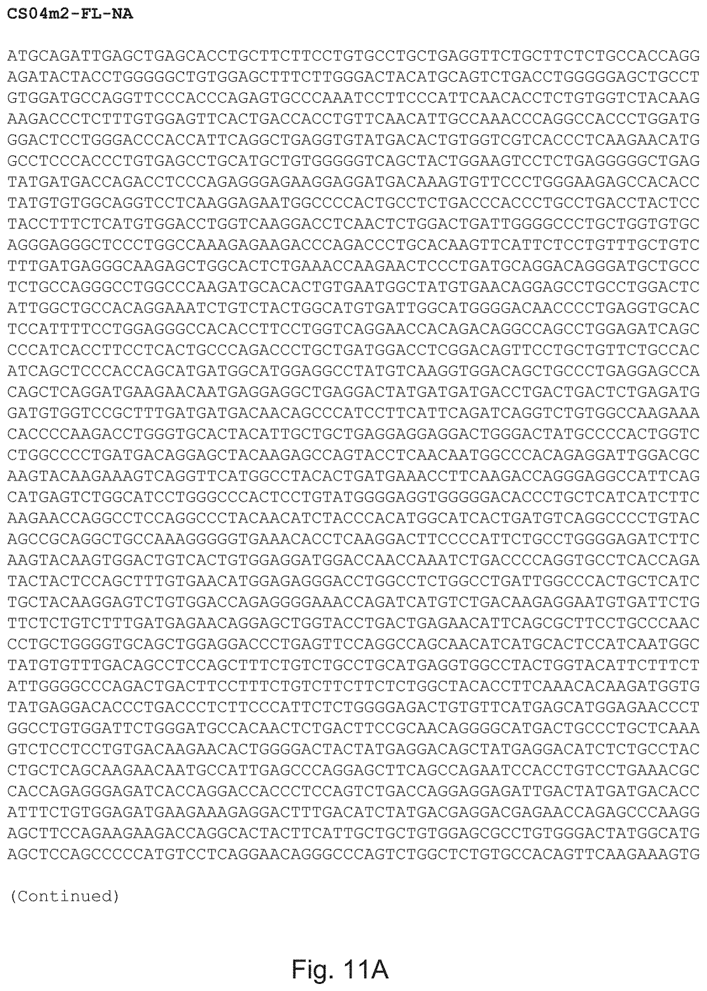

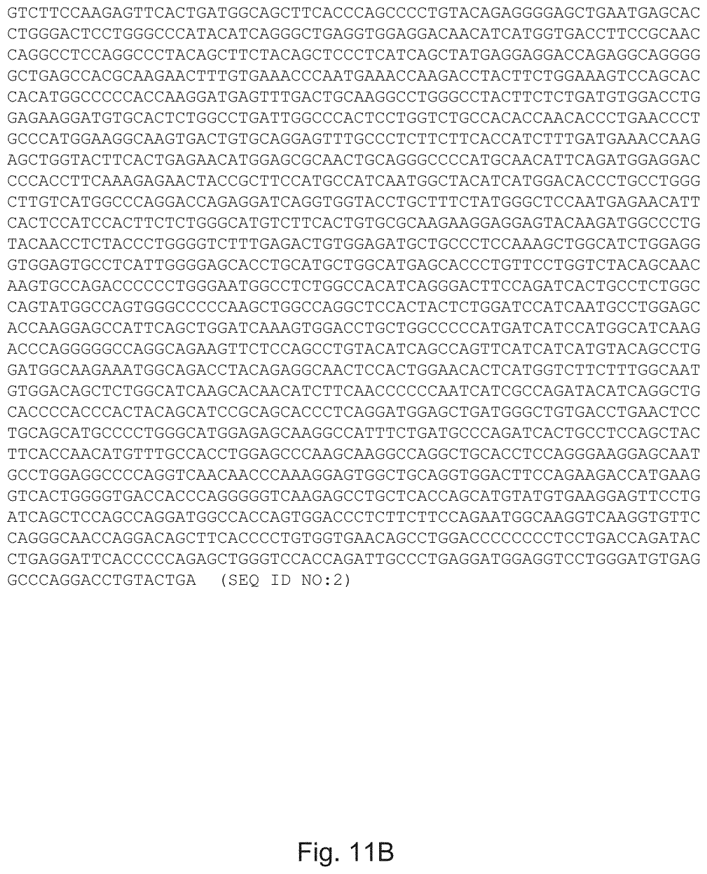

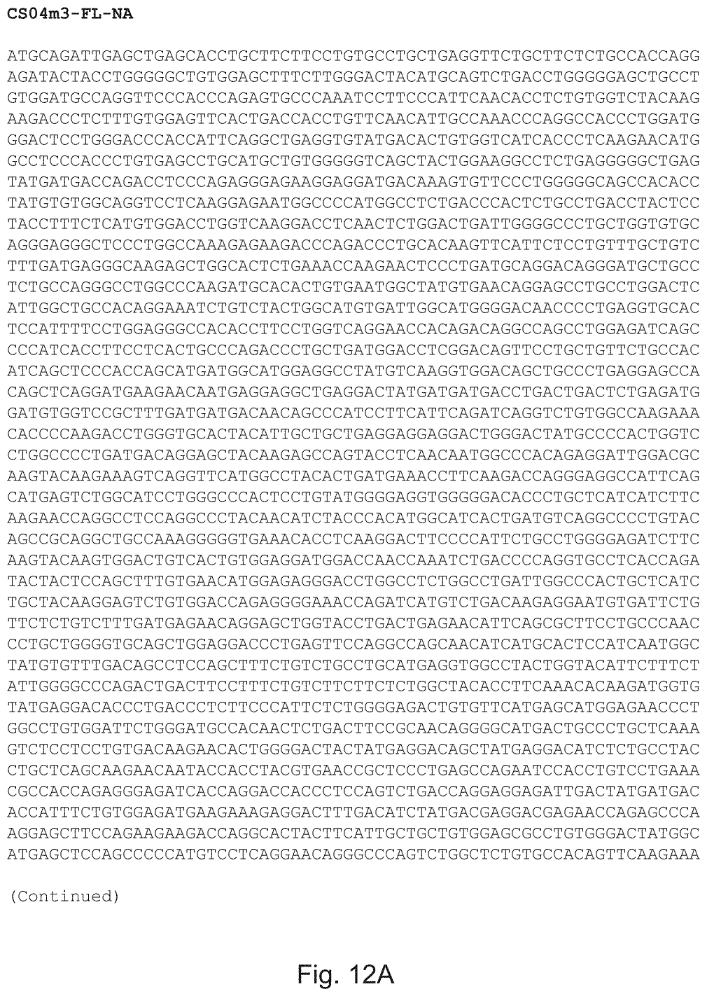

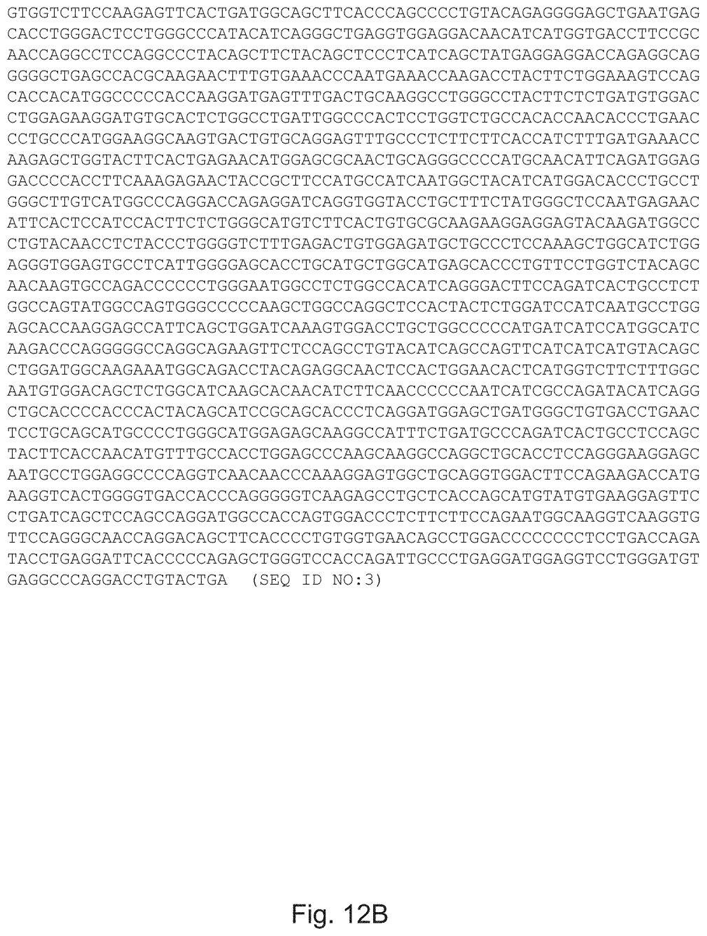

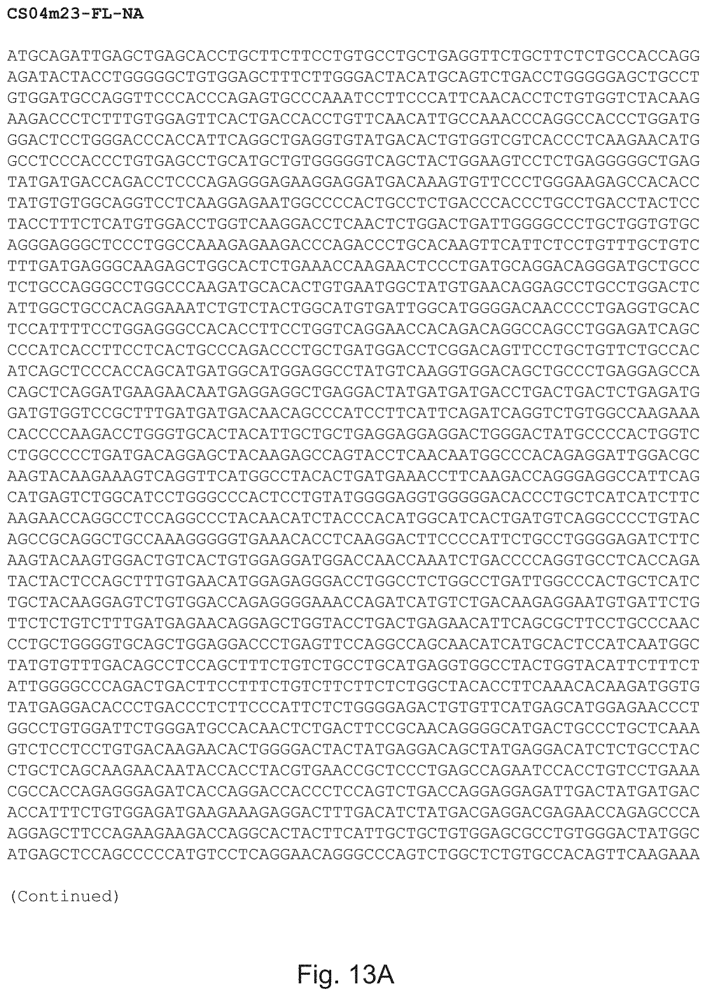

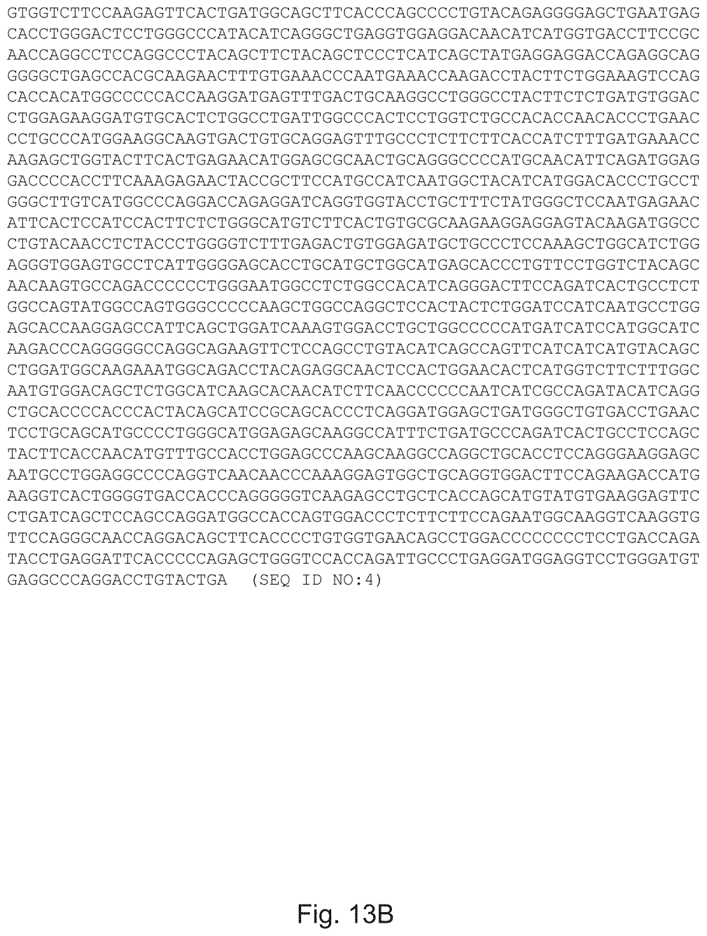

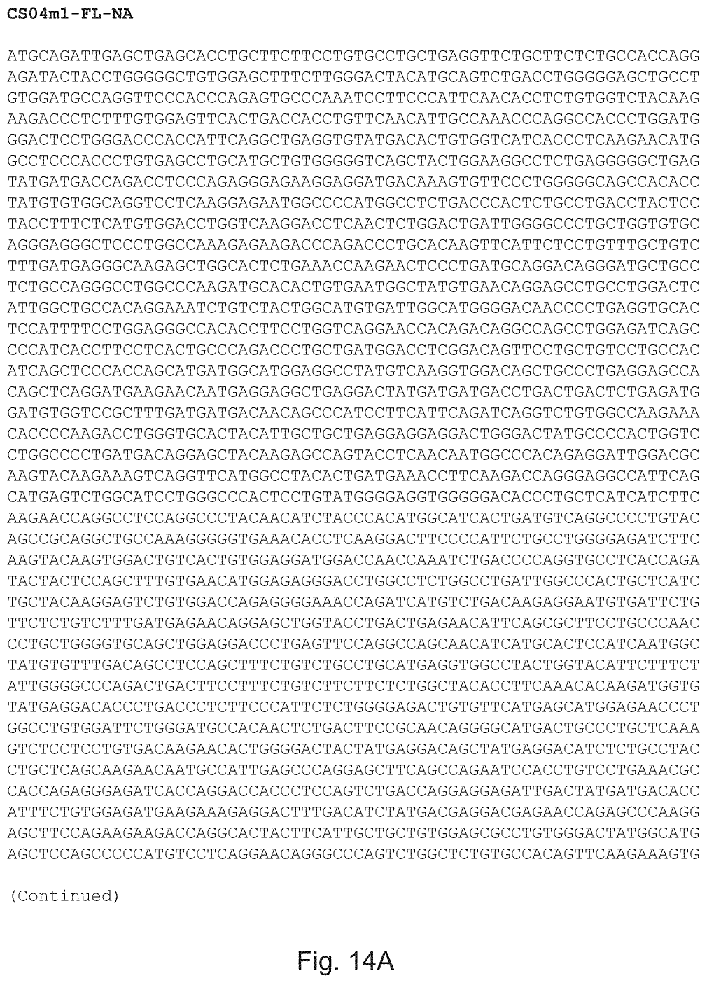

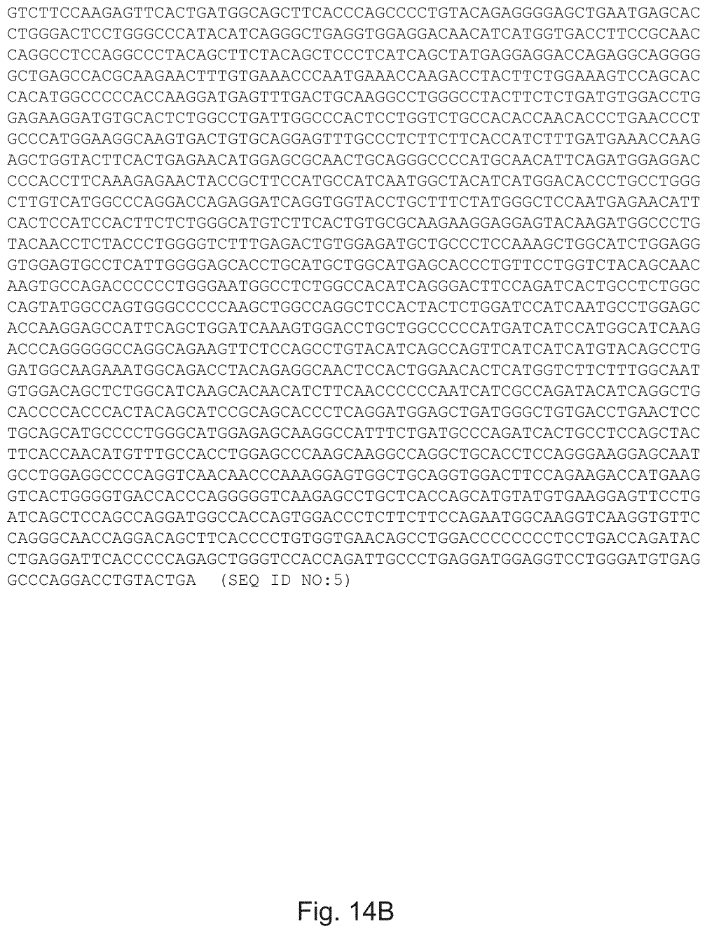

The identified variants were evaluated based on prior knowledge of the phenotypic manifestations of the known variants within identified genes, mutations as well as according to SIFT, PolyPhen-2, and Combined Annotation Dependent Depletion (CADD) algorithm values. All three methods predict deleteriousness of single nucleotide variants and insertions/deletions in the human genome. While there were no homozygous gene variants unique to the genome of patient 5, several unique heterozygous and compound heterozygous variants were found (FIG. 9). Based on current knowledge, two of the identified variants described here could potentially impact transgene expression with higher probability (FIG. 2).

One is a compound heterozygous gene variant, which involves duplications in both alleles of the SMRT/NCOR2 (“Silencing Mediator of Retinoid or Thyroid hormone receptor-2/Nuclear Receptor Co-Repressor 2”) gene, specifically p.Q508-509dup and, p.Q507-509dup. The CADD score of the identified variants indicated a moderate risk of deleteriousness (FIGS. 2 and 9). SMRT/NCoR2 is required for nuclear receptor co-repressor complex formation, which is recruited to a set of target genes via interaction with site-specific transcription factors to mediate, transcriptional silencing by recruitment of general chromatin modifying enzymes.

Another variant identified by exome sequencing was a heterozygous missense mutation variant c.344A>C, p.A115E in the IL-6R gene encoding the receptor for interleukin-6. This alanine for glutamate substitution within the IL-6 binding domain generated a CADD score of 26.1, characteristic for a very high probability of deleteriousness to IL-6 receptor function (FIGS. 2 and 9). Furthermore, haploinsufficiency of IL-6R in humans and mice has been previously documented, suggesting that the genetic variant identified in patient 5 might decrease sensitivity to IL-6-mediated inflammatory stress caused by a high load of AAV8 capsids and protect targeted hepatocytes from stress-induced death.

In this phase/II clinical dose escalation study of 8 adult male patients with hemophilia B, AAV8-based FIX Padua FIX gene therapy construct gene therapy was well-tolerated in all patients. There were no notable infusion-related or subsequent safety abnormalities, no thrombosis and no evidence of inhibitors or other FIX Padua-directed immunity during the 1-year study or subsequent follow-up. Treatment with the FIX gene therapy construct vector construct, designed to reduce immunogenic empty-AAV capsid contaminants, resulted in successful transduction of the codon-optimized FIX Padua transgene and elevated FIX expression in 7 out of 8 patients. FIX gene therapy construct administration was associated with dose-dependent increases in peak FIX:C activity that was unequivocally caused by expression of the transgene product, as demonstrated by a FIX Padua-specific ELISA. Functional FIX Padua expression in patients following FIX gene therapy construct gene therapy resulted in reductions in factor consumption, consistent with this achievement in animal studies and in patients with hemophilia B receiving a different AAV8-based FIX Padua vector.

One patient achieved sustained FIX expression with therapeutic FIX activity of ˜20% without bleeding or the use of FIX replacement therapy for >4 years. However, FIX activity was not sustained beyond 5-11 weeks in the other patients with measurable FIX Padua expression.

The acute loss of transgene expression levels within the first few months after infusion, often accompanied by an elevation in liver enzymes (exemplified by the results from patient 6), has also been reported in other AAV vector hemophilia gene therapy trials. This clinically asymptomatic hepatotoxicity (resembling autoimmune hepatitis), coincident with an acute loss in gene expression, has been attributed to a vector dose-dependent capsid-directed cellular IR. Indeed, the strongest ELISPOT and transaminase signals were measured in the highest dose cohort of this study and support the relationship to the vector dose, evident from previous trials.

In contrast to other AAV FIX gene therapy studies, where capsid-directed adaptive IRs limiting FIX expression could be managed by immunosuppression, FIX activity could not be maintained by corticosteroid prophylaxis or rescue in this study. An AAV8 FIX gene therapy study at the Children's Hospital of Philadelphia similarly reported 3 patients with vector capsid IRs that could not be rescued by immunosuppression, and a clear relationship between ALT normalization, steroid immunosuppression and the stabilization of factor expression has not been demonstrated in all patients receiving AAV gene therapy for hemophilia.

An analysis conducted to examine alternative explanations for the immunogenic loss of FIX expression with FIX gene therapy construct has provided evidence from NAb titers stimulated in mice that the higher CpG oligodeoxynucleotide (ODN) content of the FIX cDNA coding sequence in FIX gene therapy construct (introduced during codon optimization) may have contributed to increasing the immunogenicity of the vector, as lower NAb titers were developed in response to a CpG-reduced vector construct, relative to FIX gene therapy construct. Animal models of liver-directed AAV gene therapy do not predict or reproduce the CTL IRs directed against AAV seen clinically, which makes modelling these responses difficult. Thus, the validity of these models for assessing the comparative immunogenicity of similar FIX-expressing vectors is unclear.

There is firm evidence demonstrating the immunogenicity of CpGs as PAMPs signaling via TLR9 to stimulate innate IRs, which then assist in the development of strong adaptive IRs, his knowledge has been applied to the development of transgenic DNA vaccines, where CpG ODN motifs were deliberately engineered into sequences to provide an adjuvant effect. Furthermore, AAV vectors have been demonstrated to upregulate innate immune signaling through the TLR9-MyD88 pathway, and initiate adaptive IRs. Although innate immune system activation and AAV antigen presentation is thought to occur immediately after AAV vector administration, recent studies in primary human hepatocytes and a human chimeric mouse model have demonstrated a mechanism by which long-term AAV transduction triggers a later innate IR via IFN-0 that can be inactivated in order to boost AAV transduction. Taken together, this evidence suggests that the CpG-enriched FIX gene therapy construct vector could drive innate immune signaling capable of augmenting vector-targeting adaptive immunity and causing the acute loss of FIX expression observed. CpG ODN-stimulated innate immune signaling through TLR9 might be expected to initiate and maintain adaptive immunity against AAV in a more robust fashion compared with the other AAV FIX Padua vector with a lower CpG-ODN content.

Transaminitis or evidence of anti-AAV CTL activation (as measured by IFN-γ ELISPOT of PBMCs) in this study did not always predict the loss of FIX activity, especially in the lower dose cohorts, although relatively weak IRs to the vector localized in the liver might not always be detectable in assays of peripheral blood. The abrupt loss of a FIX expression peak of 58.5% activity at week 6 in patient 6 receiving highest FIX gene therapy construct dose, was concurrent with an increase in liver enzymes and PBMCs reactive to AAV8.

Transient elevation of IL-6 as an indicator for innate immune system activation was described in two different studies mapping the TLR9-dependent activation of NF-kappaB and transient pro-inflammatory cytokine induction following infusion of scAAV vector into mouse liver. Although direct experimental evidence for FIX gene therapy construct triggering an innate immune response in humans was not obtained, the variant in the IL-6R gene of patient 5, identified by sequence analysis, is suggestive of this patient being less susceptible to innate immune signaling driven by IL-6. The particular p.A115E variant identified in patient 5 has not been studied, but there is precedent for nonsynonymous IL-6R SNPs to be associated with reduced functionality. Expression of the minor allelic variant IL-6R 358Ala is specifically associated with decreased cell surface IL-6R expression, decreased sensitivity to IL-6 signalling, and protection from the development of a number of conditions with an established inflammatory component, including rheumatoid arthritis, type 1 diabetes, and coronary heart disease. The example of this specific IL-6R (gene) variant supports the notion that patient 5 might display no clinical phenotype under homeostatic conditions, yet when presented with a strong immune adjuvant, the immune response might be incomplete. In addition, inflammatory cytokines including IL-6 may also affect the degree of interaction of transcription factor HNF4a with the transthyretin (TTR) promoter and thereby modulate gene expression.

To date, no information exists about the clinical impact of the identified compound heterozygote variants of the SMRT/NCOR2 gene encoding a nuclear receptor co-repressor. SMRT/NCOR2 is required for formation of a nuclear receptor co-repressor complex that mediates transcriptional silencing of target genes via recruitment of general chromatin modifying enzymes such as histone deacetylases and methyltransferases. The SMRT complex directly interacts with HNF4α, thereby potentially directly affecting HNF4α-mediated TTR promoter expression. Taking into account that a chromatinized episomal AAV vector genome can be regulated by histone modifications, it is plausible that maintenance of the open chromatin state contributes to persistent transgene expression.

Taken together, the IL-6R variant offers a credible potential explanation for the sustained FIX expression observed uniquely in this patient, in the absence of evidence for AAV-directed IRs. This patient also highlights the influence of inter-patient variability on the success of AAV-based gene therapy that warrants further study.

Prior clinical trial experience has implicated adaptive IRs directed at the capsid as the main limiting factor for sustained expression from AAV vectors. This is the first time that innate immune stimulation, potentially driven by the CpG deoxyribonucleotide content of FIX gene therapy construct, has been implicated in the loss of gene expression from AAV vectors. These findings have led to the development of CpG-reduced vectors for gene therapy and have important implications for future clinical studies. Not only to increase vigilance for immunostimulatory components introduced during vector design, but also early control of innate immunity, which might help reduce vector immunogenicity and improve AAV transduction efficiency. Sustained therapeutic FIX expression for >4 years in patient 5, putatively linked to dysfunctional IL-6 receptor signaling, also highlights the need to understand which patient-related variables are likely to be predictive of success with current gene delivery technologies.

The persistent expression of the gene therapy vector in patient 5 indicates that either or both a mutation in the IL-6R gene associated with reduced IL-6R function or a mutation in the SMRT/NOCR2 gene associated with reduced SMRT/NCOR2 protein function is indicative of increased sensitivity of the patient to persistent infection by a viral-based transgene vector, and that a subject having one or both of these mutations may be more responsive to gene therapy than subjects without these mutations.

Thus, before deciding a treatment plan for a subject in need treatment for a disease associated with insufficient level of an enzymatic activity, it may be helpful to determine if the subject has a genotype sensitizing the subject to persistent infection by a viral-based gene therapy vector by evaluating whether the subject has one or both of a mutation in the IL-6R gene associated with reduced IL-6R function or a mutation in the SMRT/NOCR2 gene associated with reduced SMRT/NCOR2 protein function.

If it is determined that a subject has one or both of a mutation in the IL-6R gene associated with reduced IL-6R function or a mutation in the SMRT/NOCR2 gene associated with reduced SMRT/NCOR2 protein function, the subject may have a genotype sensitizing the subject to persistent infection by a viral-based gene therapy vector, and therefore, may have a greater probability of success with viral-based gene therapy. Thus, a subject determined to have one or both of a mutation in the IL-6R gene associated with reduced IL-6R function or a mutation in the SMRT/NOCR2 gene associated with reduced SMRT/NCOR2 protein function may be assigned gene therapy using the viral-based gene therapy vector.

On the other hand, if a subject is determined not to have either of a mutation in the IL-6R gene associated with reduced IL-6R function or a mutation in the SMRT/NOCR2 gene associated with reduced SMRT/NCOR2 protein function, the subject may not have a genotype sensitizing the subject to persistent infection by a viral-based gene therapy vector, and therefore, the probability of success with viral-based gene therapy may be relatively low. The subject, therefore, may not be assigned a viral-based gene therapy, and instead can be assigned an alternate therapy, such as, for example, an enzyme replacement therapy by administering a protein therapeutic having the enzymatic activity lacking in the subject.

### Example 2—Increased Immunogenicity of CpG Containing Adeno-Associated Virus Serotype 8 (AAV8) Constructs Might Contribute to the Drop of Transgene Expression

AAV8 gene therapy has shown efficacy in clinical trials. However, early spontaneous decline of transgene expression has been observed in some patients. It was hypothesized that anti-AAV8-specific T cell responses killed transduced hepatocytes resulting in a decline of transgene expression and a rise of ALT and AST levels. So far, animal models have not shown a spontaneous drop of transgene expression rendering the analysis of the vector immunogenicity on the drop of transgene activity difficult. Therefore, a murine model and 3D-bioreactor model using primary human hepatocytes was developed to assess immunogenicity of AAV8 vectors. As a first use of the models, the impact of immune-activating CpG islands in human FIX (huFIX) AAV8 vectors (AAV8-huFIX) on the immunogenicity of the vector was evaluated.

The 3D bioreactor system is an optimal system to culture primary human hepatocytes. Hence, it is used in hospitals for extracorporeal liver support. Briefly, primary human liver cells were isolated from human partial hepatectomies and cultured in the bioreactor, starting at day −3, as described in Schmelzer et al., Biotechnol Bioeng., 103(4):817-27 (2009) and Hoffmann S A, et al., Biotechnol Bioeng., 109(12):3172-81 (2012), the contents of which are incorporated herein by reference. Three days later, at day 0, primary liver cells, e.g. including Kupffer cells, were treated with an AAV8-huFIX-cpg construct containing 99 CpG islands and an AAV8-huFIX-null construct without CpGs. The cultures were terminated ten days later, at day 10, and cytokine production, liver-specific enzyme levels, and FIX gene expression were then evaluated.

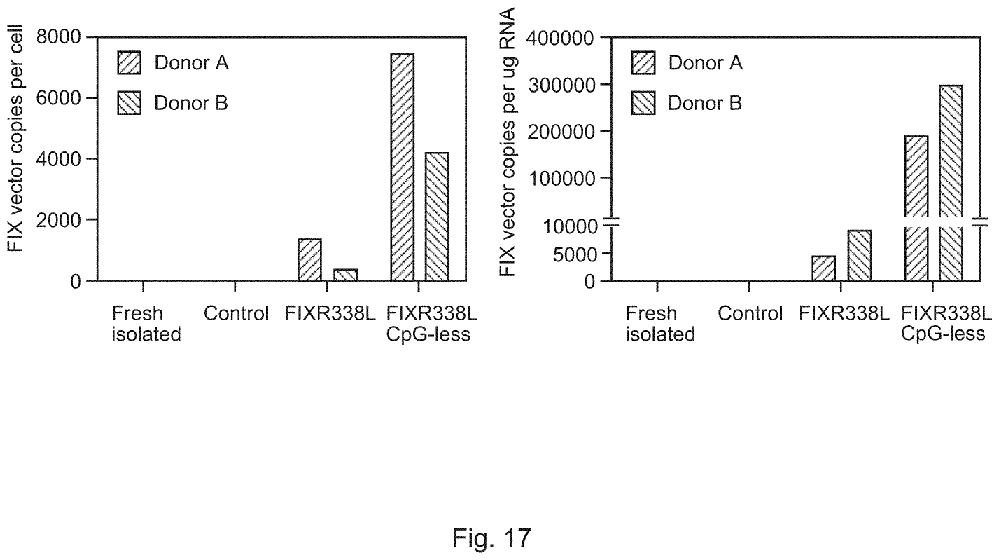

As shown in FIG. 17, AAV8-huFIX-null vectors show a higher transduction efficacy (left panel) and a higher FIX expression (right panel). In both graphs, the results from Donor A are shown on the left of the pair of bars, and the results from Donor B are shown on the right of the pair of bars.

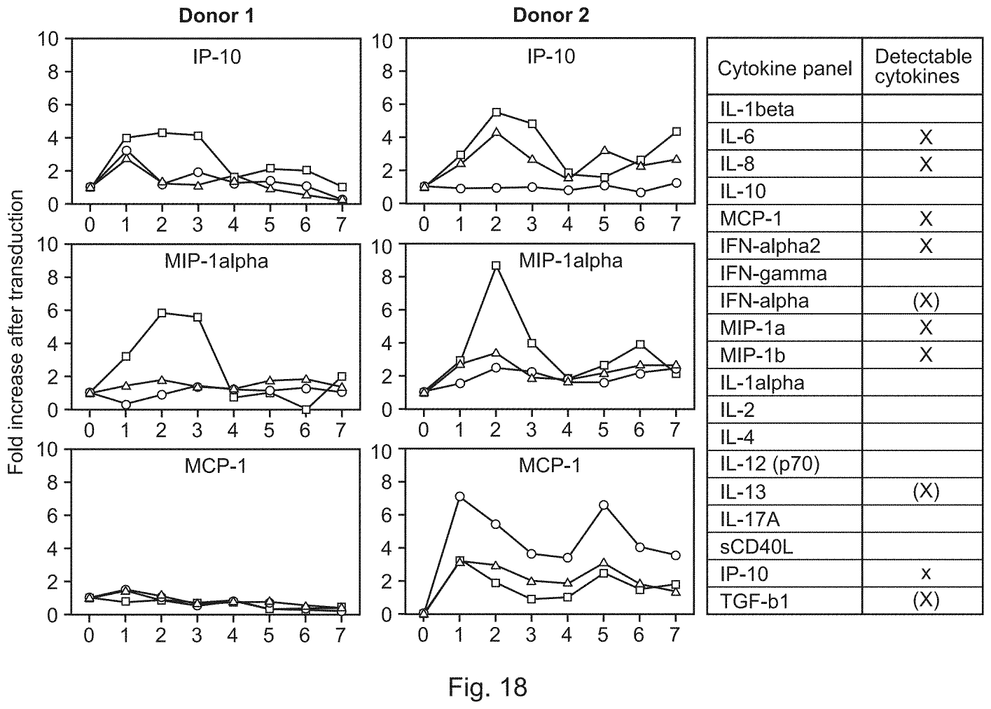

Further, the increased induction of the Th1-like cytokines IP-10 and Mip-la, as shown in FIG. 18, suggest a lower immunogenicity of AAV8-huFIX-null vectors. FIG. 18 shows a normalized time-courses of selected cytokines of two representative donors: Control bioreactors (circles) and bioreactors treated with AAV8-huFIX-cpg (squares) or AAV8-huFIX-null (triangles). Cytokine expression was overall weak, however, elevated IP-10 and Mip-1a levels were induced by AAV8-huFIX-cpg on days 2-3.

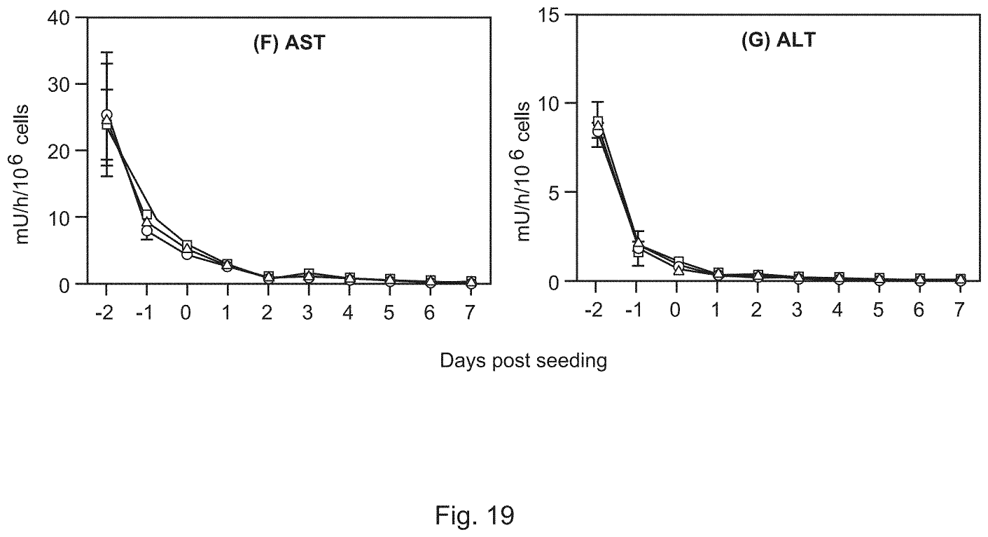

However, as shown in FIG. 19, aspartate aminotransferase (AST) and alanine aminotransferase (ALT) parameters of hepatocytes were not altered during bioreactor culturing after vector application. FIG. 19 shows AST and ALT time-courses after cell seeding: Control without infection (circles) and with infection of AAV8-huFIX-cpg (squares) or AAV8-huFIX-null (triangles). Virus-particles were applied for 24 h (day 0-day 1).

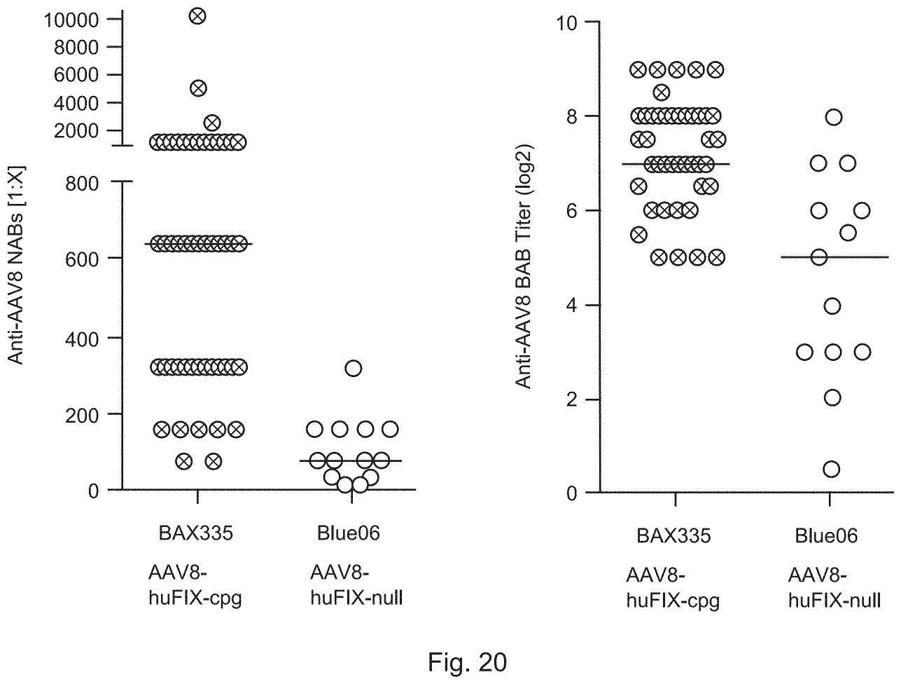

To further investigate this phenomenon in vivo, a comparative immunogenicity assessment of the AAV8-huFIX-cpg and AAV8-huFIX-null vectors were performed in mice. The capability to induce anti-AAV8 binding (BABs) and neutralizing antibodies (NABs) by innate immune activation was used as a readout. It was found that the AAV8-huFIX-null vector had lower immunogenicity than the AAV8-huFIX-cpg vector. Specifically. FIG. 20 shows that the AAV8-huFIX-cpg vector induced higher anti-AAV8 BABs (left panel) and NABs (right panel) responses than the AAV8-huFIX-null vector, suggesting a stronger activation of the TLR9 pathway by CpGs.

Taken together, these results suggest that CpG-rich AAV8-FIX constructs are more prone to activate the immune system and might contribute to the decline of transgene expression observed in some patients treated with AAV8 vectors.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method for treating a patient with viral-based gene therapy, the method comprising administering to the patient:
1) an interleukin-6 (IL6) pathway inhibitor; and
2) a viral-based gene therapy vector.

2. The method of claim 1, wherein the IL6 pathway inhibitor is an inhibitor of interleukin-6 (L6) or an inhibitor of interleukin-6 receptor (IL6R).

3. The method of claim 2, wherein the inhibitor of IL6 is an anti-IL6 monoclonal antibody.

4. The method of claim 3, wherein the anti-L6 monoclonal antibody is siltuximab, olokizumab, elsilimomab, clazakizumab, sirukumab, gerilimzumab, FM101, MEDI5117.

5. The method of claim 2, wherein the inhibitor of IL6R is an anti-IL6R monoclonal antibody.

6. The method of claim 5, wherein the anti-IL6R monoclonal antibody is tocilizumab, sarilumab, levilimab, vobarilizumab, or satralizumab.

7. The method of claim 1, wherein the IL6 pathway inhibitor is an inhibitor of the JAK/STAT3 signaling pathway, the Ras/MAPK signaling pathway, or the PI3K/Akt signaling pathway.

8. The method of claim 1, wherein the interleukin-6 (L6) pathway inhibitor is administered at least two days prior to administration of the viral-based gene therapy vector.

9. The method of claim 1, wherein the interleukin-6 (IL6) pathway inhibitor is administered at least once following administration of the viral-based gene therapy vector.

10. A method for treating a patient with viral-based gene therapy, the method comprising administering to the patient:
1) an inhibitor of the NCOR2/SMRT histone deacetylation pathway; and
2) a viral-based gene therapy vector.

11-13. (canceled)

14. The method of claim 1, wherein the viral-based gene therapy vector is an engineered adeno-associated virus (AAV) vector.

15. The method of claim 14, wherein the AAV vector is a serotype 8 AAV (AAV8) vector.

16. The method of claim 1, wherein the viral-based gene therapy vector encodes a protein selected from the group consisting of a blood coagulation factor, a serine protease, a cytokine, a soluble portion of a cytokine receptor protein, an immunoglobulin, a soluble portion of a T-cell receptor, a soluble portion of a major histocompatibility complex (MHC) protein, a complement regulatory protein, a growth factor, a soluble portion of a hormone receptor protein, a soluble portion of a cholesterol receptor protein, a transcription factor protein, and a metabolic enzyme.

17. The method of claim 1, wherein the viral-based gene therapy vector encodes a blood coagulation factor selected from the group consisting of a Factor VII polypeptide, Factor VIII polypeptide, a Factor IX polypeptide, and Factor X polypeptide.

18. The method of claim 17, wherein the viral-based gene therapy vector encodes a Factor VIII polypeptide that is a B-domain deleted Factor VIII construct.

19. The method of claim 18, wherein the B-domain deleted Factor VIII construct is encoded by a nucleic acid sequence comprising at least 95% sequence identity to a nucleic acid sequence selected from the group consisting of SEQ ID NO: 1, SEQ ID NO: 2, SEQ ID NO: 3, SEQ ID NO: 4, SEQ ID NO: 5, and SEQ ID NO: 6.

20. The method of claim 17, wherein the viral-based gene therapy vector encodes a Factor IX polypeptide tat has an R338L amino acid substitution.

21. The method of claim 20, wherein the Factor IX polypeptide is encoded by a nucleic acid sequence of SEQ ID NO: 7.

22. The method claim 1, wherein the viral-based gene therapy vector encodes a protein selected from the group consisting of granulocyte-macrophage colony-stimulating factor (GM-CSF), retinoid isomerohydrolase (RPE65), survival of motor neuron 1 (SMN1).

23. The method claim 1, wherein the viral-based gene therapy vector is selected from the group consisting of talimogene laherparepvec, voretigene neparvovec-rzyl, and onasemnogene abeparvovec-xioi.

24-32. (canceled)

33. A method for treating a patient with viral-based gene therapy, the method comprising administering to the patient:
1) an interleukin-6 (IL6) pathway inhibitor;
2) a viral-based gene therapy vector; and
3) a corticosteroid at a dose of no more than 20 mg per day.

34-35. (canceled)

36. A method for treating a patient with viral-based gene therapy, the method comprising:
determining whether the patient has a genotype sensitizing the patient to persistent infection by a viral-based gene therapy vector by one or both of:
evaluating whether the patient has a mutation in the SMRT/NCOR2 gene associated with reduced SMRT/NCOR2 protein function, and
evaluating whether the patient has a mutation in the interleukin-6 receptor (IL-6R) gene associated with reduced IL-6R function; and
if the patient has either a mutation in the SMRT/NCOR2 gene associated with reduced SMRT/NCOR2 protein function or a mutation in the IL-6R gene associated with reduced IL-6R function, administering a viral-based gene therapy vector to the patient.

37-47. (canceled)


SEQUENCE LISTING (14 sequences)

SEQ ID NO: 1 - DNA, 4374 bp, Artificial Sequence
  Synthetic polynucleotide
atgcagattgagctgagcacctgcttcttcctgtgcctgctgaggttctgcttctctgccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcatcaccctcaagaacatggcctcccaccctgtgagcctgcatgctgtgggggtcagctactggaaggcctctgagggggctgagtatgatgaccagacctcccagagggagaaggaggatgacaaagtgttccctgggggcagccacacctatgtgtggcaggtcctcaaggagaatggccccatggcctctgacccactctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgttctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaatgccattgagcccaggagcttcagccagaatccacctgtcctgaaacgccaccagagggagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtactga

SEQ ID NO: 2 - DNA, 4374 bp, Artificial Sequence
  Synthetic polynucleotide
atgcagattgagctgagcacctgcttcttcctgtgcctgctgaggttctgcttctctgccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcgtcaccctcaagaacatggcctcccaccctgtgagcctgcatgctgtgggggtcagctactggaagtcctctgagggggctgagtatgatgaccagacctcccagagggagaaggaggatgacaaagtgttccctgggaagagccacacctatgtgtggcaggtcctcaaggagaatggccccactgcctctgacccaccctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgttctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaatgccattgagcccaggagcttcagccagaatccacctgtcctgaaacgccaccagagggagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtactga

SEQ ID NO: 3 - DNA, 4377 bp, Artificial Sequence
  Synthetic polynucleotide
atgcagattgagctgagcacctgcttcttcctgtgcctgctgaggttctgcttctctgccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcatcaccctcaagaacatggcctcccaccctgtgagcctgcatgctgtgggggtcagctactggaaggcctctgagggggctgagtatgatgaccagacctcccagagggagaaggaggatgacaaagtgttccctgggggcagccacacctatgtgtggcaggtcctcaaggagaatggccccatggcctctgacccactctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgttctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaataccacctacgtgaaccgctccctgagccagaatccacctgtcctgaaacgccaccagagggagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtactga

SEQ ID NO: 4 - DNA, 4377 bp, Artificial Sequence
  Synthetic polynucleotide
atgcagattgagctgagcacctgcttcttcctgtgcctgctgaggttctgcttctctgccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcgtcaccctcaagaacatggcctcccaccctgtgagcctgcatgctgtgggggtcagctactggaagtcctctgagggggctgagtatgatgaccagacctcccagagggagaaggaggatgacaaagtgttccctgggaagagccacacctatgtgtggcaggtcctcaaggagaatggccccactgcctctgacccaccctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgttctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaataccacctacgtgaaccgctccctgagccagaatccacctgtcctgaaacgccaccagagggagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtactga

SEQ ID NO: 5 - DNA, 4374 bp, Artificial Sequence
  Synthetic polynucleotide
atgcagattgagctgagcacctgcttcttcctgtgcctgctgaggttctgcttctctgccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcatcaccctcaagaacatggcctcccaccctgtgagcctgcatgctgtgggggtcagctactggaaggcctctgagggggctgagtatgatgaccagacctcccagagggagaaggaggatgacaaagtgttccctgggggcagccacacctatgtgtggcaggtcctcaaggagaatggccccatggcctctgacccactctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgtcctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaatgccattgagcccaggagcttcagccagaatccacctgtcctgaaacgccaccagagggagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtactga

SEQ ID NO: 6 - DNA, 4377 bp, Artificial Sequence
  Synthetic polynucleotide
atgcagattgagctgagcacctgcttcttcctgtgcctgctgaggttctgcttctctgccaccaggagatactacctgggggctgtggagctttcttgggactacatgcagtctgacctgggggagctgcctgtggatgccaggttcccacccagagtgcccaaatccttcccattcaacacctctgtggtctacaagaagaccctctttgtggagttcactgaccacctgttcaacattgccaaacccaggccaccctggatgggactcctgggacccaccattcaggctgaggtgtatgacactgtggtcatcaccctcaagaacatggcctcccaccctgtgagcctgcatgctgtgggggtcagctactggaaggcctctgagggggctgagtatgatgaccagacctcccagagggagaaggaggatgacaaagtgttccctgggggcagccacacctatgtgtggcaggtcctcaaggagaatggccccatggcctctgacccactctgcctgacctactcctacctttctcatgtggacctggtcaaggacctcaactctggactgattggggccctgctggtgtgcagggagggctccctggccaaagagaagacccagaccctgcacaagttcattctcctgtttgctgtctttgatgagggcaagagctggcactctgaaaccaagaactccctgatgcaggacagggatgctgcctctgccagggcctggcccaagatgcacactgtgaatggctatgtgaacaggagcctgcctggactcattggctgccacaggaaatctgtctactggcatgtgattggcatggggacaacccctgaggtgcactccattttcctggagggccacaccttcctggtcaggaaccacagacaggccagcctggagatcagccccatcaccttcctcactgcccagaccctgctgatggacctcggacagttcctgctgtcctgccacatcagctcccaccagcatgatggcatggaggcctatgtcaaggtggacagctgccctgaggagccacagctcaggatgaagaacaatgaggaggctgaggactatgatgatgacctgactgactctgagatggatgtggtccgctttgatgatgacaacagcccatccttcattcagatcaggtctgtggccaagaaacaccccaagacctgggtgcactacattgctgctgaggaggaggactgggactatgccccactggtcctggcccctgatgacaggagctacaagagccagtacctcaacaatggcccacagaggattggacgcaagtacaagaaagtcaggttcatggcctacactgatgaaaccttcaagaccagggaggccattcagcatgagtctggcatcctgggcccactcctgtatggggaggtgggggacaccctgctcatcatcttcaagaaccaggcctccaggccctacaacatctacccacatggcatcactgatgtcaggcccctgtacagccgcaggctgccaaagggggtgaaacacctcaaggacttccccattctgcctggggagatcttcaagtacaagtggactgtcactgtggaggatggaccaaccaaatctgaccccaggtgcctcaccagatactactccagctttgtgaacatggagagggacctggcctctggcctgattggcccactgctcatctgctacaaggagtctgtggaccagaggggaaaccagatcatgtctgacaagaggaatgtgattctgttctctgtctttgatgagaacaggagctggtacctgactgagaacattcagcgcttcctgcccaaccctgctggggtgcagctggaggaccctgagttccaggccagcaacatcatgcactccatcaatggctatgtgtttgacagcctccagctttctgtctgcctgcatgaggtggcctactggtacattctttctattggggcccagactgacttcctttctgtcttcttctctggctacaccttcaaacacaagatggtgtatgaggacaccctgaccctcttcccattctctggggagactgtgttcatgagcatggagaaccctggcctgtggattctgggatgccacaactctgacttccgcaacaggggcatgactgccctgctcaaagtctcctcctgtgacaagaacactggggactactatgaggacagctatgaggacatctctgcctacctgctcagcaagaacaataccacctacgtgaaccgctccctgagccagaatccacctgtcctgaaacgccaccagagggagatcaccaggaccaccctccagtctgaccaggaggagattgactatgatgacaccatttctgtggagatgaagaaagaggactttgacatctatgacgaggacgagaaccagagcccaaggagcttccagaagaagaccaggcactacttcattgctgctgtggagcgcctgtgggactatggcatgagctccagcccccatgtcctcaggaacagggcccagtctggctctgtgccacagttcaagaaagtggtcttccaagagttcactgatggcagcttcacccagcccctgtacagaggggagctgaatgagcacctgggactcctgggcccatacatcagggctgaggtggaggacaacatcatggtgaccttccgcaaccaggcctccaggccctacagcttctacagctccctcatcagctatgaggaggaccagaggcagggggctgagccacgcaagaactttgtgaaacccaatgaaaccaagacctacttctggaaagtccagcaccacatggcccccaccaaggatgagtttgactgcaaggcctgggcctacttctctgatgtggacctggagaaggatgtgcactctggcctgattggcccactcctggtctgccacaccaacaccctgaaccctgcccatggaaggcaagtgactgtgcaggagtttgccctcttcttcaccatctttgatgaaaccaagagctggtacttcactgagaacatggagcgcaactgcagggccccatgcaacattcagatggaggaccccaccttcaaagagaactaccgcttccatgccatcaatggctacatcatggacaccctgcctgggcttgtcatggcccaggaccagaggatcaggtggtacctgctttctatgggctccaatgagaacattcactccatccacttctctgggcatgtcttcactgtgcgcaagaaggaggagtacaagatggccctgtacaacctctaccctggggtctttgagactgtggagatgctgccctccaaagctggcatctggagggtggagtgcctcattggggagcacctgcatgctggcatgagcaccctgttcctggtctacagcaacaagtgccagacccccctgggaatggcctctggccacatcagggacttccagatcactgcctctggccagtatggccagtgggcccccaagctggccaggctccactactctggatccatcaatgcctggagcaccaaggagccattcagctggatcaaagtggacctgctggcccccatgatcatccatggcatcaagacccagggggccaggcagaagttctccagcctgtacatcagccagttcatcatcatgtacagcctggatggcaagaaatggcagacctacagaggcaactccactggaacactcatggtcttctttggcaatgtggacagctctggcatcaagcacaacatcttcaaccccccaatcatcgccagatacatcaggctgcaccccacccactacagcatccgcagcaccctcaggatggagctgatgggctgtgacctgaactcctgcagcatgcccctgggcatggagagcaaggccatttctgatgcccagatcactgcctccagctacttcaccaacatgtttgccacctggagcccaagcaaggccaggctgcacctccagggaaggagcaatgcctggaggccccaggtcaacaacccaaaggagtggctgcaggtggacttccagaagaccatgaaggtcactggggtgaccacccagggggtcaagagcctgctcaccagcatgtatgtgaaggagttcctgatcagctccagccaggatggccaccagtggaccctcttcttccagaatggcaaggtcaaggtgttccagggcaaccaggacagcttcacccctgtggtgaacagcctggacccccccctcctgaccagatacctgaggattcacccccagagctgggtccaccagattgccctgaggatggaggtcctgggatgtgaggcccaggacctgtactga

SEQ ID NO: 7 - DNA, 1386 bp, Artificial Sequence
  Synthetic polynucleotide
atgcagagggtcaacatgatcatggctgagtcccctggcctcatcaccatctgcctgctgggctacctgctgtctgctgagtgcactgtcttcctggaccatgagaatgccaacaagatcctcaacaggcccaagagatacaactctggcaaactggaggagtttgtccagggcaacctggagagggagtgcatggaggagaagtgctcctttgaggaggccagggaggtctttgagaacactgagcgcaccactgagttctggaaacagtatgtggatggggaccagtgtgagtccaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgctcctgcactgagggctaccgcctggctgagaaccagaagagctgtgagcctgctgtgccattcccatgtggcagagtctctgtgagccagaccagcaagctcaccagggctgagactgtgttccctgatgtggactatgtgaacagcactgaggctgaaaccatcctggacaacatcacccagagcacccagagcttcaatgacttcaccagagtggtgggaggagaggatgccaagcctggccagttcccctggcaagtggtgctcaatggcaaggtggatgccttctgtgggggctccattgtgaatgagaagtggattgtcactgctgcccactgtgtggagactggggtcaagatcactgtggtggctggggagcacaacattgaggagactgagcacactgagcagaagcgcaatgtgatcaggatcatcccccaccacaactacaatgctgccatcaacaagtacaaccatgacattgccctgctggagctggatgagcccctggtcctcaacagctatgtgacccccatctgcattgctgacaaggagtacaccaacatcttcctcaagtttggctctggctatgtctctggctggggcagagtgttccacaaaggcaggtctgccctggtgctccagtacctgagagtgcccctggtggacagggccacctgcctcttgagcaccaagttcaccatctacaacaacatgttctgtgctggcttccatgagggaggaagagacagctgccagggggactctggaggaccccatgtcactgaggtggagggcacctccttcctcactggcatcatctcctggggagaggagtgtgccatgaaaggcaaatatggcatctacaccaaagtctccagatatgtcaactggatcaaggagaagaccaagctgacctga

SEQ ID NO: 8 - protein, 7 aa, Artificial Sequence
  Synthetic peptide
PQLRMKN

SEQ ID NO: 9 - protein, 7 aa, Artificial Sequence
  Synthetic peptide
VDQRGNQ

SEQ ID NO: 10 - protein, 7 aa, Artificial Sequence
  Synthetic peptide
AIEPRSF

SEQ ID NO: 11 - protein, 8 aa, Artificial Sequence
  Synthetic peptide
TTYVNRSL

SEQ ID NO: 12 - protein, 14 aa, Artificial Sequence
  Synthetic peptide
SFSQNPPVLKRHQR

SEQ ID NO: 13 - protein, 24 aa, Artificial Sequence
  Synthetic peptide
SFAQNSRPPSASAPKPPVLRRHQR

SEQ ID NO: 14 - protein, 16 aa, Artificial Sequence
  Synthetic peptide
SFSQNSRHQAYRYRRG